(19) United States
(12) Patent Application Publication
(10) Pub. No.: US 2024/0360442 A1
(43) Pub. Date: Oct. 31, 2024

(54) COMPOSITIONS AND METHODS FOR MODULATING GENE TRANSCRIPTION NETWORKS

(71) Applicant: Nuclear RNA Networks, Inc., Danville, CA (US)

(72) Inventor: Melanie Adams, Danville, CA (US)

(73) Assignee: Nuclear RNA Networks, Inc., Danville, CA (US)

(21) Appl. No.: 18/307,000

(22) Filed: Apr. 25, 2023

Related U.S. Application Data
(63) Continuation of application No. PCT/US22/17371, filed on Feb. 22, 2022.
(60) Provisional application No. 63/151,222, filed on Feb. 19, 2021.

(51) Int. Cl. C12N 15/113; G16B 30/10

(52) U.S. Cl. CPC C12N 15/113; G16B 30/10

(57) ABSTRACT

The invention involves the use of novel nucleic acid sequences to detect, modulate, ablate, inhibit or augment the transcription and therefore translation and expression of functionally-linked genes. The present disclosure is based on the novel finding that Transposable Element remnant (TEr) RNA or promoter non-processive transcripts (NPtx) have a high probability of aligning with high identity to transcriptional regulatory regions of functionally-linked genes, suggesting that they participate in beneficial transcriptional crosstalk.

### CROSS REFERENCE TO RELATED APPLICATIONS

This application claims priority to U.S. Provisional Patent Application No. 63/151,222, filed Feb. 19, 2021, which is hereby incorporated by reference. REFERENCE TO A “SEQUENCE LISTING,” A TABLE, OR A COMPUTER PROGRAM, LISTING APPENDIX SUBMITTED ON A COMPACT DISK

The instant application contains a Sequence Listing which has been submitted electronically in XML file format and is hereby incorporated by reference in its entirety. Said XML file, created on Aug. 10, 2023, is named 129443-5001-US Sequence Listing.xml and is 3.82 MB in size.

### BACKGROUND OF THE INVENTION

Transposable elements (TE, “jumping genes”) are now recognized as drivers of evolutionary innovation in gene transcription, both disrupting and dispersing transcription factor binding sites (TFBS) when they transpose. (Miller W J, McDonald J F, Pinsker W. Molecular domestication of mobile elements. Genetica. 1997; 100(1-3):261-70; Pehrsson E C, Choudhary M N K, Sundaram V, Wang T. The epigenomic landscape of transposable elements across normal human development and anatomy. Nature Communications. 2019; 10(1):5640; Lowe C B, Bejerano G, Haussler D. Thousands of human mobile element fragments undergo strong purifying selection near developmental genes. Proceedings of the National Academy of Sciences. 2007; Johnson R, Guigó R. The RIDL hypothesis: Transposable elements as functional domains of long noncoding RNAs. RNA. 2014; Bourque G, Leong B, Vega V B, Chen X, Lee Y L, Srinivasan K G, et al. Evolution of the mammalian transcription factor binding repertoire via transposable elements. Genome Res. 2008; 18(11):1752-62; Chuong E B, Elde N C, Feschotte C. Regulatory activities of transposable elements: From conflicts to benefits. 2017). However, the astonishing bulk of TE sequences in the human genome is thought to be accumulated residua; a functional role for the cell type-specific TE remnant (TEr) RNAs that are transcribed in all tissues and cell lines tested to date is mostly unknown. (Hall L L, Carone D M, Gomez A V, Kolpa H J, Byron M, Mehta N, et al. Stable C0T-1 repeat RNA is abundant and is associated with euchromatic interphase chromosomes. Cell. 2014; Camevali D, Conti A, Pellegrini M, Dieci G. Whole-genome expression analysis of mammalian-wide interspersed repeat elements in human cell lines. DNA research: an international journal for rapid publication of reports on genes and genomes. 2017; Xie M, Hong C, Zhang B, Lowdon R F, Xing X, Li D, et al. DNA hypomethylation within specific transposable element families associates with tissue-specific enhancer landscape. Nature Genetics. 2013; Johnson J M, Edwards S, Shoemaker D, Schadt E E. Dark matter in the genome: Evidence of widespread transcription detected by microarray tiling experiments. 2005; Chishima T, Iwakiri J, Hamada M. Identification of transposable elements contributing to tissue-specific expression of long non-coding RNAs. Genes. 2018.) Adding to their status as genomic “junk”, TE replication involves the duplication of DNA, or reverse transcription of TE RNA into complimentary DNA, and nucleotide substitution errors can occur or adjacent DNA or RNA sequences incorporated, resulting in the majority of TEs harboring sequence polymorphisms. (Malone C D, Hannon G J. Small RNAs as Guardians of the Genome. 2009; Villanueva-Cañas J L, Rech G E, de Cara M A R, González J. Beyond SNPs: how to detect selection on transposable element insertions. Methods in Ecology and Evolution. 2017; Umylny B, Presting G, Efird J T, Klimovitsky B I, Ward W S. Most human Alu and murine B1 repeats are unique. Journal of Cellular Biochemistry. 2007).

Uniquely tested by the inventor was the common assumption that the small sequence variation that allows determination of the genomic position of a repetitive element is physiologically irrelevant “junk”. Surprisingly, results suggest that protein-to-protein networks are mirrored by direct gene-to-gene networks between the genes that encode them, through the sharing of high identity “junk” DNA sequences. The unexpected specificity of this “junk” indicates its potential role in guidance of epigenetic chromatin-modifying complexes between functionally-linked genes by TEr-primed Argonautes and TEr-containing lncRNA. In addition, results suggest a new model of disease pathogenesis in which mis-regulation of TEr transcripts leads to aberrant guidance of transcription effector-complexes between the genes that share complementary partners, creating a transcription “network-opathy”. Results presented herein indicate that this may be the case in certain forms of Parkinson's disease. In vitro data confirms the predictive value of the methods disclosed herein in designing a molecule that is a powerful modulator of epithelial to mesenchymal transition.

The NPtx and TEr sequences have not otherwise been classified as miRNA, piRNA, siRNA, eRNA or other RNA of known function. Shared high-identity sequences ranged in length from 20 bp to hundreds of base pairs. They were sometimes transcribed in cell-type specific patterns into small RNA fragments unrelated to transposition. They were often found in lncRNA. Alignments were not pericentromeric and rarely in 3′UTR of coding-genes. All TE families and subtypes were represented in percentages consistent with their reported frequency in the human genome.

The invention includes nucleic acid sequences that are predicted to detect, modulate, ablate, inhibit or augment the transcription and therefore translation and expression of functionally-linked genes in phospholipid signaling-mediated cell activation, epithelial to mesenchymal transition, Parkinson's disease, myogenesis, stress-related fat metabolism and Tₕ-immune cell activation.

### SUMMARY

In an aspect, the present disclosure provides for the use of one or more Transposable Element remnant (TEr) nucleic acid sequences and promoter and promoter-proximal non-processive transcripts (NPtx) sequences of pathway hub genes and/or their associated (in cis or trans) lncRNA, to augment, alter, block or otherwise modify the transcription of genes that contain high identity (but not necessarily identical) nucleic acid sequences.

In another aspect, the present disclosure provides for a method to identify the DNA sequences of one or more Transposable Element remnant (TEr) nucleic acids and promoter and promoter-proximal non-processive transcripts (NPtx) of pathway hub genes.

In another aspect, the present disclosure provides for specific nucleic acid sequences that can be utilized to block, disrupt or augment one or more of the following pathways: 1) epithelial to mesenchymal transition, 2) phospholipid signaling pathway, 3) myogenesis, 4) Parkinson's Disease-associated pathways, 5) stress-mediated fat metabolism, 6) CD4+ T cell activation and HIV binding, wherein the nucleic acid sequences have sequence identifiers provided herein.

In another aspect, the present disclosure provides for nucleic acid sequences provided herein further modified by the addition of nuclear localization signals and/or “bar codes” and/or other nucleic acid identifiers and/or other synthetic modifiers.

In another aspect, the present disclosure provides for a composition comprising a nucleic acid sequences disclosed herein, and delivery molecule comprising viral vectors, nanoparticles or extracellular vesicles.

In another aspect, the present disclosure provides for a use of sequences provided herein as diagnostic or prognostic tool.

In another aspect, the present disclosure provides for a use of sequences provided herein to define a tumor or disease signature.

In another aspect, the present disclosure provides for the use of sequences provided herein for inhibition of epithelial to mesenchymal transition and/or maintaining tumor heterogeneity.

In another aspect, the present disclosure provides for the use of sequences provided herein for identification of cell function-specific pathways and/or for staging specific differentiation or developmental stages in cells, tissue and/or tissue samples.

In another aspect, the present disclosure provides for the use of sequences provided herein to trigger or modify stem cells to differentiate into a tissue and/or cell type-of-interest and/or inducing specific differentiation or developmental stages in cells, tissue and/or tissue samples.

In another aspect, the present disclosure provides for the use of TEr/NPtx-specific stands that are discovered by “pulled down” techniques, including but not restricted to Chromatin Immunoprecipitation for example, for the further identification of a specific genomic pathway or network.

In another aspect, the present disclosure provides for a synthetic nucleic acid comprising one or more of a transposon remnant, a promoter and/or a promoter-proximal non-processive transcript, selected to modulate gene-to-gene transcriptional signaling within a given functional pathway.

In another aspect, the present disclosure provides for a method of modulating epigenetic communication between genes coordinating specific pathways, comprising: delivering one or more synthetic nucleic acids as provided herein to a sample of cells and/or a tissue and/or an animal model of disease and/or a human clinical trial.

In another aspect, the present disclosure provides for a method of determining a network of genes, comprising the steps of:
(a) selecting a transposon remnant, a promoter, or a promoter-proximal non-processive transcript of a first index gene from a given functional pathway;
(b) identifying, using a computer implemented sequence alignment algorithm implemented by a processor, transposon remnant sequences from a set of genes, having at least 75% homology with the selected transposon remnant, promoter, or promoter-proximal non-processive transcript;
(c) determining, by the processor, a genomic position of the transposon remnant sequences with highest sequence identity with the selected transposon remnant, promoter, or promoter-proximal non-processive transcript;
(d) in response to a determination that the genomic position of a given identified transposon remnant sequence is within a gene regulatory region of a first gene among the set of genes, tabulating, by the processor, function of the first gene;
(e) repeating (a)-(d) for identified transposon remnant sequences that are in cis to the selected transposon remnant, promoter, or promoter-proximal non-processive transcript to determine transposon remnant sequences of genes connected to the first index gene; and
(f) repeating (a)-(e) with transposon remnant sequences of genes, among the set of genes, connected to the first index gene to determine a group of genes forming the given functional pathway.

In another aspect, the present disclosure provides for inducing specific differentiation or developmental stages in cells, comprising:
determining a group of genes forming a given functional pathway using any of the methods described herein;
delivering one or more synthetic nucleic acids comprising one or more of a transposon remnant, a promoter and/or a promoter-proximal non-processive transcript, and selected to modulate gene-to-gene transcriptional signaling within the given functional pathway,
wherein the given functional pathway is associated with the specific differentiation or developmental stages in cells.

### BRIEF DESCRIPTION OF THE DRAWINGS

FIG. 1. TE disperse highly specific variant sequences (“siblings”) to small groups of genes that are conserved within functionally-linked genes if they participate in transcriptional “crosstalk” that is evolutionarily beneficial. The ability of transposition to disperse small groups of high-identity TE variants (“siblings”) suggested the hypothesis that remnants of these siblings could participate in precise gene-to-gene transcriptional crosstalk based on shared nucleic acid sequences of high identity, unrelated to their transcription factor DNA binding sites or TE subtype-specific RNA secondary structure.

FIG. 2. TEr, NPtx and other “junk” non-processive RNA transcripts prime nuclear Argonaute/chromatin modifying complexes to DNA loci that are expressing complementary sequence.

FIG. 3. Exonic TEr guide lncRNA that scaffolds and chaperones transcription factors to DNA loci that are expressing complementary sequence.

FIG. 4A-4B. The model predicts neural-like networks will form between functionally-linked genes. 4a) each TEr is a small rate-limiting step to transcription of the full-length mRNA, a rate limiting step determined by the expression of its complementary sequence in trans; 4b) NFkB1/RELA TEr Network as an example of an Artificial Neural Network formed by TEr-mediated transcriptional crosstalk. The system is sensitive to shifts in 3D gene spacing and concentration of the TEr sequences, determined in turn by the transcription rate of their host gene. A threshold number of epigenetic modifications to TEr are required for processive (completed) transcription of any one gene. Genes can crosstalk at TEr “network nodes”, without necessarily leading to processive transcription of the full gene. Results suggest a new model of disease pathogenesis in which mis-regulation of TEr transcripts leads to aberrant guidance of transcription effector-complexes between the genes that share complementary partners, creating a transcription “network-opathy”.

FIG. 5. Evolutionary evidence that the model sheds light on a process whereby random distribution of TEr siblings could result in highly specific gene networks. The highly conserved MIR remnant within the FAK promoters of Human, xenopus and Murine species aligned to EMT-critical genes, but to different ones.

FIG. 6. The role of piRNA/PIWI in germ cells may be more than the silencing of transposing, and therefore mutagenic, transposons. TEr that have contributed to the evolution of multi-cellularity and tissue differentiation could also be placed “on hold” (quiescent) by piRNA-PIWI complexes, rather than terminally silenced, allowing their reactivation as necessary for embryogenesis and tissue-specific gene regulation.

FIG. 7. How Index TE are chosen. Example of Index TEr chosen within a conserved regulatory region of the NFkB1 enhancer.

FIG. 8. Flowchart of discovery algorithm using UCSC Genome Browser on Human December 2013 (GRCh38/hg38).

FIG. 9. Example of sequence alignment showing regions identified by BLAT2013 as high identity to NFkB1 AluJr_{Zebrafish} (position shown in FIG. 7, conserved to Zebrafish, ˜550 million yrs). NOTE: These aligned sequences are dispersed by TEr “siblings” (FIG. 1) and are termed “Core Template Sequences”.

FIG. 10. Summary of statistical analysis.

FIG. 11. Graphic representation of the statistically significant alignment results for Index TEr of the muscle/cardiovascular system. Significant fractions of mm/CVS index TE BLAT₂₀₁₃ top ten alignments were to other genes with Muscle/Cardiovascular Function, as compared to IS index TE (P<0.008 t test) or DEV index TE (P<0.008).

FIG. 12. Phospholipid Signaling Pathway genes aligned by NFkB1 and lncRNA_{LOC105377621/RP11-499E18.1} TEr sequences. The ancient Phospholipid Signaling Pathway is initiated by inflammatory and proliferative signals that activate cell membrane phospholipids, triggering immediate intracellular release of Ca²⁺ and the phosphorylation of effector proteins that activate NFkB1 (outlined in FIG. 15). Multiple genes encoding isoforms of key proteins critical to the initiation of phospholipid signaling were aligned by NFkB TEr including PI3-Kinase (PI3K-C2A), Phospholipase A (PLA2G4A) and Phospholipase C (PLC-E1). TEr with high identity to genes of this pathway were present throughout KFkB1 transcriptional regulatory regions including its upstream lncRNA_{LOC105377621/RP11-499E18.1} (highlighted by *). PLC-E1 was aligned by two different Alu Repeats in the promoter-proximal region of NFkB1 intron 1: AluYa5 and AluSz6_{chr4:102507477-102507601} (which also aligned KSR2, see below). Index TEr aligned to three genes encoding enzyme isoforms responsible for Phosphatidic Acid (PA) metabolism to DAG (Diacylglycerol Kinase Iota, Kappa and Eta; DGKI, DGKK and DGKH; and aligned another gene of this same pathway twice: TAMM41 (Mitochondrial Translocator Assembly and Maintenance Homolog; catalyzes the reaction of PA to CDP-diacylglycerol (CDP-DAG).

FIG. 13. Examples of TEr of NFkB1 and cis lncRNA_{LOC105377621/RP11-499E18.1} that align genes that define specific cellular pathways: genes of the Phospholipid Signaling Pathway (pink), genes of the RAS signaling pathway (red) and genes of epithelial to mesenchymal transition (green).

FIG. 14. NFkB1 has five NFkB1 TEr sequences that align with high identity to four genes encoding RAS inhibitors (KSR2 is aligned twice). TEr that align to KSR2 and NF-1 are adjacent to each other on NFkB1 intron 1 and are both “hub” regulators of the Ras signal transduction pathway.

FIG. 15. The network of functionally-linked genes is extended into same phospholipid signaling pathway by NFkB1/KSR2 “sibling” AluSz TEr alignments. Interestingly, the sibling AluSz in KSR2 also aligns to with high-identity to PRR5 (Proline Rich 5; hormone sensitive mTORC2 subunit, modulates PKC-Alpha). The original NFkB1 AluSz is adjacent to a TEr that aligned “PRR5-Like”. It is highly unlikely that these results would occur randomly. A brief outline of the Phospholipid Signaling Pathway is also shown. Proteins highlighted in red circles have isoforms aligned by NFkB1 TEr and their siblings.

FIG. 16. Adjacent promoter-proximal TEr in NFkB1 intron 1 align to genes critical to the initiation of EMT at the plasma membrane: LTBP1 (Latent-Transforming Growth Factor Beta-Binding Protein 1), LGR5 (Leucine-Rich Repeat-Containing G-Protein Coupled Receptor 5), LRP5L (Low Density Lipoprotein Receptor-Related Protein 5-Like), CTNNA3 (Catenin (Cadherin-Associated Protein), Alpha 3). LTBP1 is aligned twice: by TEr of NFkB1intron 1 and lncRNA_{Loco5377621/RP11-499E18.1}. Both NFkB1 and lncRNA_{LOC105377621/RP11-499E18.1} TEr align an isoform of FNBP1, critical to the formation of Adherens Junctions and cell-to-cell adhesion. GPC5 and 6 are surface heparan sulfate proteoglycans; GPC5 enhances migration and invasion of cancer cells through WNT5A signaling and among GPC6 related pathways is phospholipase-C.

FIG. 17: Tissue expression of NFkB1 and lncRNA_{LOC105377621/RP11-499E18.1} (isoforms termed LOC105377621 by UCSC are here termed LOC621“a” and RP11-499E18.1 is here termed LOC621“b-c”) and genes repeatedly aligned by both. Tissue expression is high in brain, lung and cultured fibroblasts (ENCODE₂₀₁₃ RNAseq). Definition of aligned proteins is presented in Table 8.

FIG. 18: RNAseq analysis of NFkB1 and lncRNA_{LOC105377621/RP11-499E18.1} in pancreatic adenocarcinoma cell lines (GSE88759). NFkB1 and lncRNA_{LOC105377621/RP11-499E18.1} were expressed in a well differentiated (epithelial) pancreatic cancer cell line (BxPC3) and silenced in a poorly differentiated (mesenchymal) cell line (S2-007/Suit2) suggesting their loss is associated with tumor progression. Red circle highlights expressed regions of lncRNA_{LOC105377621} and blue circles highlight expressed regions of NFkB1 intron 1.

FIG. 19. RP11-499E18.1 isoforms contain exonic TEr. The predominant isoforms (LOC621c) initiate with an AluY, which is usually spliced to a fragment of an AluSc. All isoforms terminate with MTL1J.

FIG. 20. SiRNA-mediated knock down (KD) designed for RP11-499E18.1 resulted in progression of the well differentiated human pancreatic adenocarcinoma cell line BxPC3 from epithelial to mesenchymal phenotype

FIG. 21. SiRNA-mediated KD of RP11-499E18.1 in human metastasizing pancreatic adenocarcinoma Suit2 cells resulted in transition of mixed population of both adherent spindling cells and poorly-differentiated small round cells into predominantly small round cells with no apparent contact-inhibition

FIG. 22. SiRNA-mediated knock down of RP11-499E18.1 in human metastasizing pancreatic adenocarcinoma COL0357 cells resulted in transition of the nested epithelioid cells into erratic small nests of small cells which, when stimulated with TGFb, enlarged and lost all signs of cell-to-cell contact. While responding to TGFb, the cells look nothing like the TGFb-stimulated mesenchymal/spindling cells of the control

FIG. 23. Highly expressed in muscle myoblasts, MyoD1 TEr and its upstream lncRNA_{RP11-358H18.3} have a high likelihood of aligning muscle-specific genes. Results unlikely to be random included MyoD1 TEr alignments to RYR2 (aligned twice, by different TEr) and RYR3 (ryanodine receptor 2, 3; calcium channels required specifically for muscle cell contraction: cardiac (isoform 2) and skeletal (isoform 3); highlighted in red). MN1 transcriptional regulator (ubiquitously expressed; highest median expression in Muscle-Skeletal) was also aligned twice, as was C10orf71 (Open Reading Frame70; unknown function, highly expressed solely in skeletal muscle). Similar to TEr of coding gene NFkB1 and its cis lncRNA_{LOC105377621/RP11-499E18.1} (both of which aligned EMT pathway-specific genes), MyoD1 upstream cis lncRNA_{LOC102723330/RP11-358H18.3} contained TEr that aligned to critical genes of myogenesis (highlighted in blue). For example, exon 2 MIRc (conserved to Xenopus) aligned with high identity to CDON1 (Cell Adhesion Associated, Oncogene Regulated 1; mediates cell-cell interactions between muscle precursor cells and positively regulates myogenesis) and Vasoactive Intestinal Peptide (VIP; stimulates myocardial contractility and causes vasodilation. Extended MyoD1 3′ UTR loci not otherwise notated as lncRNA consisted of highly transcribed TEr. Genes essential to myogenesis were aligned by these TEr as well. LncRNA_{LINCO2729} is expressed in testes only.

FIG. 24. The L2b initiating transcription from Steroid Receptor RNA Activator 1 (SRA1) has a high likelihood of aligning genes associated with Parkinson's Disease.

FIG. 25. Location of non-processive “junk” transcripts (NPtx) and lncRNA AF213884.3 within NFkB1 promoter that share high-identity TEr with genes participating in formation, processing, packaging and function of mRNA (Table 10).

FIG. 26. Summary of EMT initiation by Wnt, b-Catenin and FAK/PTK2 signaling.

FIG. 27. Genes participating in the Epithelial to Mesenchymal Transition that aligned with high sequence identity to b-Catenin promoter TEr sequence.

FIG. 28. Genes participating in the Epithelial to Mesenchymal Transition that aligned with high sequence identity to Wnt10B/1 shared promoter TEr sequence.

FIG. 29. Flowchart highlighting EMT pathway genes aligned by promoter TEr of FAK, b-Catenin, Wnt10B,1 and Wnt2.

FIG. 30. Intron 1 MER21C of CRHR2 aligns an endocrine-mediated gene network that participates in lipid metabolism. The STRING database (protein:protein interactions) highlights the finding of pathway-specific proteins discovered by TEr sequence genomic alignments.

FIG. 31. Graphical Abstract: results suggest that protein-to-protein networks are mirrored by direct gene-to-gene networks between the genes that encode them through the sharing of high identity “junk” DNA sequences. Given ancient mechanisms by which nucleic acid complementarity (RNA-mediated epigenetic mechanisms which allow precision in RNA/DNA-mediated signaling and targeting of proteins) our results suggest complex gene-to-gene communication networks can be identified, traced and therapeutically modified using the “junk” sequences that have been duplicated and dispersed by transposons for millennia.

FIG. 32A-32IIII. Sequences for TE templates for various index genes and corresponding portions of sequences having high identity with an aligned gene.
SEQ ID NOS: 1-7 are TE template sequences for NFkB1 template L1PB1 range=chr4:102464307-102464661.
SEQ ID NOS:8-19 are TE template sequences for NFkB1 template L1M6 range=chr4:102464705-102465277.
SEQ ID NOS:20-23 are TE template sequences for NFkB1 template AluJr range=chr4:102465811-102465981.
SEQ ID NOS:23-26 are TE template sequences for NFkB1 template AluJr ange=chr4:102466015-102466135.
SEQ ID NOS:27-49 are TE template sequences for NFkB1 template L1PB1 range=chr4:102459784-102460950.
SEQ ID NOS:50-76 are TE template sequences for NFkB1 template L1PB1 range=chr4:102458176-102459486.
SEQ ID NOS:77-81 are TE template sequences for NFkB1 template L1PBa1 range=chr4:102460951-102461180.
SEQ ID NOS:82-90 are TE template sequences for NFkB1 template MSTC range=chr4:102456262-102456665.
SEQ ID NOS:91-94 are TE template sequences for NFkB1 template MLT1K range=chr4:102457054-102457327.
SEQ ID NOS:95-100 are TE template sequences for NFkB1 template AluSq2 range=chr4:102459487-102459783.
SEQ ID NOS:101-104 are TE template sequences for NFkB1 template L1M6 range=chr4:102457972-102458156.
SEQ ID NOS: 105-113 are TE template sequences for NFkB1 template LTR16A2 range=chr4:102457329-102457742.
SEQ ID NOS:114-117 are TE template sequences for NFkB1 template MamGypLTR1c range=chr4:102456686-102456865.
SEQ ID NOS: 118-119 are TE template sequences for NFkB1 template LTR81B range=chr4:102454134-102454208.
SEQ ID NOS: 120-123 are TE template sequences for NFkB1 template LTR81B range=chr4:102453693-102453809.
SEQ ID NOS:124-126 are TE template sequences for NFkB1 template FLAM_A range=chr4:102469163-102469262.
SEQ ID NOS: 127-131 are TE template sequences for NFkB1 template MIRb range=chr4:102469431-102469661.
SEQ ID NOS:132-139 are TE template sequences for NFkB1 template MLT1A0 range=chr4:102468399-102468755.
SEQ ID NOS: 140-160 are TE template sequences for NFkB1 template L1MD1 range=chr4:102470492-102471503.
SEQ ID NOS: 161-162 are TE template sequences for NFkB1 template MIR3 range=chr4:102452674-102452739.
SEQ ID NOS:163-165 are TE template sequences for NFkB1 template MamRTE1 range=chr4:102451994-102452097.
SEQ ID NOS: 166-167 are TE template sequences for NFkB1 template L1M6 range=chr4:102469266-102469330.
SEQ ID NOS:168-199 are TE template sequences for NFkB1 template MLT1A0-int range=chr4:102466803-102468398.
SEQ ID NOS:200-205 are TE template sequences for NFkB1 template A1uSx1 range=chr4:102499715-102499995.
SEQ ID NOS:206-215 are TE template sequences for NFkB1 template MLT1C range=chr4:102498997-102499448.
SEQ ID NOS:216-224 are TE template sequences for NFkB1 template MSTB1 range=chr4:102498326-102498742.
SEQ ID NOS:225-228 are TE template sequences for NFkB1 template MIR range=chr4:102497855-102498045.
SEQ ID NOS:229-238 are TE template sequences for NFkB1 template L2 range=chr4:102497231-102497825.
SEQ ID NOS:239-246 are TE template sequences for NFkB1 template MLT1B range=chr4:102496240-102496617.
SEQ ID NOS:247-249 are TE template sequences for NFkB1 template MER81 range=chr4:102496090-102496191.
SEQ ID NOS:250-256 are TE template sequences for NFkB1 template L1MEj range=chr4:102493931-102494278.
SEQ ID NOS:257-313 are TE template sequences for NFkB1 template L1PB1 range=chr4:102485859-102488680.
SEQ ID NOS:314-336 are TE template sequences for NFkB1 template L1PA6 range=chr4:102484657-102485768.
SEQ ID NOS:337-371 are TE template sequences for NFkB1 template LTR12C range=chr4:102482956-102484656.
SEQ ID NOS:372-472 are TE template sequences for NFkB1 template L1PA6 range=chr4:102477934-102482955.
SEQ ID NOS:473-475 are TE template sequences for NFkB1 template L1PA6 range=chr4:103619161-103619277.
SEQ ID NOS:476-477 are TE template sequences for NFkB1 template L2a range=chr4:102505799-102505857.
SEQ ID NOS:478-480 are TE template sequences for NFkB1 template A1uSz6 range=chr4:102507477-102507601.
SEQ ID NOS:481-485 are TE template sequences for NFkB1 template HAL1ME range=chr4:102510807-102511027.
SEQ ID NOS:486-488 are TE template sequences for NFkB1 template L1MA9 range=chr4:102511116-102511227.
SEQ ID NOS:489-491 are TE template sequences for NFkB1 template L2a range=chr4:102511254-102511361.
SEQ ID NOS:492-498 are TE template sequences for NFkB1 template A1uJo range=chr4:102511394-102511703.
SEQ ID NOS:499-502 are TE template sequences for NFkB1 template L1MAB range=chr4:102511709-102511897.
SEQ ID NOS:503-509 are TE template sequences for NFkB1 template AluJr range=chr4:102512340-102512644.
SEQ ID NOS:510-515 are TE template sequences for NFkB1 template AluY range=chr4:102513892-102514190.
SEQ ID NOS:516-521 are TE template sequences for NFkB1 template AluYa5 range=chr4:102515108-102515409.
SEQ ID NOS:522-525 are TE template sequences for NFkB1 promoter non-processive transcripts range=chr4:102499993-102500159.
SEQ ID NOS:526-533 are portions of template sequences for NFkB1 template L1PB1 range=chr4:102464307-102464661 having a high identity with MCC (ENST00000408903.6) gene.
SEQ ID NOS:534-541 are portions of template sequences for NFkB1 template L1PB1 range=chr4:102464307-102464661 having a high identity with HECW2 (ENST00000260983.8) gene.
SEQ ID NOS:542-549 are portions of template sequences for NFkB1 template L1PB1 range=chr4:102464307-102464661 having a high identity with CD2AP (ENST00000359314.5) gene.
SEQ ID NOS:550-557 are portions of template sequences for NFkB1 template L1PB1 range=chr4:102464307-102464661 having a high identity with AFF2 (ENST00000370460.6) gene.
SEQ ID NOS:558-565 are portions of template sequences for NFkB1 template L1PB1 range=chr4:102464307-102464661 having a high identity with KLHDC2 (ENST00000298307.9) gene.
SEQ ID NOS:566-573 are portions of template sequences for NFkB1 template LlPB1 range=chr4:102464307-102464661 having a high identity with RORB (ENST00000376896.7) gene.
SEQ ID NO:574 is a portion of template sequence for NFkB1 template L1PB1 range=chr4:102464307-102464661 having a high identity with CTNNBIP1 (ENST00000377263.6) gene.
SEQ ID NO:575 is a portion of template sequence for NFkB1 template L1PB1 range=chr4:102464307-102464661 having a high identity with ELOA-AS1 (ENST00000655402.1) gene.
SEQ ID NO:576 is a portion of template sequence for NFkB1 template L1PB1 range=chr4:102464307-102464661 having a high identity with SSX2IP (ENST00000342203.7) gene.
SEQ ID NO:577 is a portion of template sequence for NFkB1 template L1M6 range=chr4:102464705-102465277 having a high identity with ANXA7 (ENST00000372921.9) gene.
SEQ ID NO:578 is a portion of template sequence for NFkB1 template L1M6 range=chr4:102464705-102465277 having a high identity with PLA2G4A (ENST00000367466.3) gene.
SEQ ID NO:579-582 are portions of template sequence for NFkB1 template AluJr range=chr4:102465811-102465981 having a high identity with TMIGD1 (ENST00000538566.6) gene.
SEQ ID NO:583-585 are portions of template sequence for NFkB1 template AluJr range=chr4:102465811-102465981 having a high identity with RNF111 (ENST00000348370.8) gene.
SEQ ID NO:586-593 are portions of template sequence for NFkB1 template AluJr range=chr4:102465811-102465981 having a high identity with SMG1P2 (NR_135305.1) gene.
SEQ ID NO:594-596 are portions of template sequence for NFkB1 template AluJr range=chr4:102466015-102466135 having a high identity with PIK3C2A (RefSeq: NM_001321378.1) gene.
SEQ ID NO:597-599 are portions of template sequence for NFkB1 template AluJr range=chr4:102466015-102466135 having a high identity with FNBP1L (ENST00000260506.12) gene.
SEQ ID NO:600-602 are portions of template sequence for NFkB1 template AluJr range=chr4:102466015-102466135 having a high identity with PHF11 (ENST00000378319.7) gene.
SEQ ID NO:603-626 are portions of template sequence for NFkB1 template L1PB1 range=chr4:102459784-102460950 having a high identity with KCNH1 (ENST00000367007.5) gene.
SEQ ID NO:627-650 are portions of template sequence for NFkB1 template L1PB1 range=chr4:102459784-102460950 having a high identity with CA3-AS1 (ENST00000517697.5) gene.
SEQ ID NO:651-676 are portions of template sequence for NFkB1 template LiPB1 range=chr4:102458176-102459486 having a high identity with CA3-AS1 (ENST00000517697.5) gene.
SEQ ID NO:677-702 are portions of template sequence for NFkB1 template L1PB1 range=chr4:102458176-102459486 having a high identity with PDE7A (ENST00000401827.7) gene.
SEQ ID NO:703-728 are portions of template sequence for NFkB1 template L1PB1 range=chr4:102458176-102459486 having a high identity with MUSK (ENST00000374448.8) gene.
SEQ ID NO:729-755 are portions of template sequence for NFkB1 template L1PB1 range=chr4:102458176-102459486 having a high identity with DGKI (ENST00000453654.6) gene.
SEQ ID NO:756-760 are portions of template sequence for NFkB1 template L1PBa1 range=chr4:102460951-102461180 having a high identity with DGKK (ENST00000611977.1) gene.
SEQ ID NO:761-765 are portions of template sequence for NFkB1 template L1PBa1 range=chr4:102460951-102461180 having a high identity with DDX11-AS1 (ENST00000500527.1) gene.
SEQ ID NO:766-774 are portions of template sequence for NFkB1 template MSTC range=chr4:102456262-102456665 having a high identity with POLR3E (ENST00000615879.4) gene.
SEQ ID NO:775-776 are portions of template sequence for NFkB1 template MSTC range=chr4:102456262-102456665 having a high identity with AP002992.1 (ENST00000530842.2) gene.
SEQ ID NO:777-782 are portions of template sequence for NFkB1 template AluSq2 range=chr4:102459487-102459783 having a high identity with MED11 (ENST00000575284.5) gene.
SEQ ID NO:783-788 are portions of template sequence for NFkB1 template AluSq2 range=chr4:102459487-102459783 having a high identity with SCAI (ENST00000336505.10) gene.
SEQ ID NO:789-794 are portions of template sequence for NFkB1 template AluSq2 range=chr4:102459487-102459783 having a high identity with ITFG1 (ENST00000320640.10) gene.
SEQ ID NO:795-800 are portions of template sequence for NFkB1 template AluSq2 range=chr4:102459487-102459783 having a high identity with MAPKAP1 (ENST00000373511.6) gene.
SEQ ID NO:801 is a portion of template sequence for NFkB1 template AluSq2 range=chr4:102459487-102459783 having a high identity with CTNNA1 (ENST00000627109.2) gene.
SEQ ID NO:802 is a portion of template sequence for NFkB1 template L1M6 range=chr4:102457972-102458156 having a high identity with IMPA1 (ENST00000256108.9) gene.
SEQ ID NO:803 is a portion of template sequence for NFkB1 template LTR16A2 range=chr4:102457329-102457742 having a high identity with ESRRB (ENST00000512784.6) gene.
SEQ ID NO:804 is a portion of template sequence for NFkB1 template MamGypLTR1c range=chr4:102456686-102456865 having a high identity with CALN1 (ENST00000329008.9) gene.
SEQ ID NO:805-806 are portions of template sequence for NFkB1 template LTR81B range=chr4:102454134-102454208 having a high identity with GPC6 (ENST00000377047.8) gene.
SEQ ID NO:807 is a portion of template sequence for NFkB1 template LTR81B range=chr4:102453693-102453809 having a high identity with SEMA4A (ENST00000355014.6) gene.
SEQ ID NO:808 is a portion of template sequence for NFkB1 template LTR81B range=chr4:102453693-102453809 having a high identity with FMN1 (ENST00000616417.4) gene.
SEQ ID NO:809-811 are portions of template sequence for NFkB1 template LTR81B range=chr4:102453693-102453809 having a high identity with SDK1 (ENST00000404826.6) gene.
SEQ ID NO:812 is a portion of template sequence for NFkB1 template LTR81B range=chr4:102453693-102453809 having a high identity with PAK1 (ENST00000356341.7) gene.
SEQ ID NO:813 is a portion of template sequence for NFkB1 template LTR81B range=chr4:102453693-102453809 having a high identity with NFIA (ENST00000371191.5) gene.
SEQ ID NO:814 is a portion of template sequence for NFkB1 template FLAM_A range=chr4:102469163-102469262 having a high identity with WTIP (ENST00000590071.6) gene.
SEQ ID NO:815-816 are portions of template sequence for NFkB1 template FLAM_A range=chr4:102469163-102469262 having a high identity with TBC1D1 (ENST00000261439.8) gene.
SEQ ID NO:817-819 are portions of template sequence for NFkB1 template FLAM_A range=chr4:102469163-102469262 having a high identity with TBC1D3P5 (NR_033892.1) gene.
SEQ ID NO:820-822 are portions of template sequence for NFkB1 template FLAM_A range=chr4:102469163-102469262 having a high identity with KSR1 (ENST00000644974.1) gene.
SEQ ID NO:823-825 are portions of template sequence for NFkB1 template MIRb range=chr4:102469431-102469661 having a high identity with PRICKLE2 (ENST00000638394.1) gene.
SEQ ID NO:826 is a portion of template sequence for NFkB1 template MIRb range=chr4:102469431-102469661 having a high identity with PARP9 (ENST00000477522.6) gene.
SEQ ID NO:827 is a portion of template sequence for NFkB1 template MIRb range=chr4:102469431-102469661 having a high identity with RFTN2 (ENST00000295049.8) gene.
SEQ ID NO:828 is a portion of template sequence for NFkB1 template MIRb range=chr4:102469431-102469661 having a high identity with ADCY9 (ENST00000294016.7) gene.
SEQ ID NO:829 is a portion of template sequence for NFkB1 template MIRb range=chr4:102469431-102469661 having a high identity with NCOA1 (ENST00000406961.5) gene.
SEQ ID NO:830-835 are portions of template sequence for NFkB1 template MLT1A0 range=chr4:102468399-102468755 having a high identity with OTOA (ENST00000646100.1) gene.
SEQ ID NO:836-840 are portions of template sequence for NFkB1 template MLT1A0 range=chr4:102468399-102468755 having a high identity DUSP27 (ENST00000361200.6) gene.
SEQ ID NO:841-846 are portions of template sequence for NFkB1 template MLT1A0 range=chr4:102468399-102468755 having a high identity with DUSP27 (ENST00000361200.6) gene.
SEQ ID NO:847-856 are portions of template sequence for NFkB1 template L1MD1 range=chr4:102470492-102471503 having a high identity with ATP10B (XM_011534468.2) gene.
SEQ ID NO:857-864 are portions of template sequence for NFkB1 template L1MD1 range=chr4:102470492-102471503 having a high identity with MED13L (ENST00000281928.8) gene.
SEQ ID NO:865-883 are portions of template sequence for NFkB1 template MLT1A0-int range=chr4:102466803-102468398 having a high identity with KLHL40 (ENST00000287777.4) gene.
SEQ ID NO:884-889 are portions of template sequence for NFkB1 template AluSx1 range=chr4:102499715-102499995 having a high identity with UNKL (ENST00000389221.8) gene.
SEQ ID NO:890-895 are portions of template sequence for NFkB1 template AluSx1 range=chr4:102499715-102499995 having a high identity with GPATCH3 (ENST00000361720.9) gene.
SEQ ID NO:896-902 are portions of template sequence for NFkB1 template MLT1C range=chr4:102498997-102499448 having a high identity with DCAF17 (ENST00000375255.7) gene.
SEQ ID NO:903-908 are portions of template sequence for NFkB1 template MLT1C range=chr4:102498997-102499448 having a high identity with ADGRL3 (ENST00000512091.6) gene.
SEQ ID NO:909-915 are portions of template sequence for NFkB1 template MSTB1 range=chr4:102498326-102498742 having a high identity with MTMR1 (ENST00000370390.7) gene.
SEQ ID NO:916-923 are portions of template sequence for NFkB1 template MLT1C range=chr4:102498997-102499448 having a high identity with PRR5L (ENST00000530639.5) gene.
SEQ ID NO:924 is a portion of template sequence for NFkB1 template MIR range=chr4:102497855-102498045 having a high identity with INPP5D (ENST00000359570.9) gene.
SEQ ID NO:925 is a portion of template sequence for NFkB1 template MIR range=chr4:102497855-102498045 having a high identity with MIR3681HG (ENST00000451644.5) gene.
SEQ ID NO:926 is a portion of template sequence for NFkB1 template L2 range=chr4:102497231-102497825 having a high identity with SCAI (ENST00000336505.10) gene.
SEQ ID NO:927-933 are portions of template sequence for NFkB1 template MLT1B range=chr4:102496240-102496617 having a high identity with IL10RA (ENST00000227752.7) gene.
SEQ ID NO:934-940 are portions of template sequence for NFkB1 template MLT1B range=chr4:102496240-102496617 having a high identity with FAM89A (ENST00000366654.4) gene.
SEQ ID NO:941-942 are portions of template sequence for NFkB1 template MER81 range=chr4:102496090-102496191 having a high identity with IFT52 (ENST00000373030.7) gene.
SEQ ID NO:943 is a portion of template sequence for NFkB1 template L1MEj range=chr4:102493931-102494278 having a high identity with DCAF6 (ENST00000432587.6) gene.
SEQ ID NO:944-955 are portions of template sequence for NFkB1 template L1PB1 range=chr4:102485859-102488680 having a high identity with EGLN1 (ENST00000366641.3) gene.
SEQ ID NO:956-1006 are portions of template sequence for NFkB1 template L1PB1 range=chr4:102485859-102488680 having a high identity with NRG1 (ENST00000519301.5) gene.
SEQ ID NO:1007-1062 are portions of template sequence for NFkB1 template L1PB1 range=chr4:102485859-102488680 having a high identity with WARS2 (ENST00000369426.9) gene.
SEQ ID NO:1063-1084 are portions of template sequence for NFkB1 template L1PB1 range=chr4:102485859-102488680 having a high identity with KSR2 (ENST00000425217.5) gene.
SEQ ID NO:1085-1106 are portions of template sequence for NFkB1 template L1PB1 range=chr4:102485859-102488680 having a high identity with RPAP3 (ENST00000005386.7) gene.
SEQ ID NO:1107-1141 are portions of template sequence for NFkB1 template LTR12C range=chr4:102482956-102484656 having a high identity with NPBWR1 (ENST00000331251.3) gene.
SEQ ID NO:1142-1242 are portions of template sequence for NFkB1 template L1PA6 range=chr4:102477934-102482955 having a high identity with KSR2 (ENST00000425217.5) gene.
SEQ ID NO:1243-1343 are portions of template sequence for NFkB1 template L1PA6 range=chr4:102477934-102482955 having a high identity with SENP6 (ENST00000370010.6) gene.
SEQ ID NO:1344-1444 are portions of template sequence for NFkB1 template L1PA6 range=chr4:102477934-102482955 having a high identity with CD207 (XM_011532876.2) gene.
SEQ ID NO:1445-1447 are portions of template sequence for NFkB1 template L1PA6 range=chr4:103619161-103619277 having a high identity with TAMM41 (ENST00000623275.3) gene.
SEQ ID NO:1448-1450 are portions of template sequence for NFkB1 template L1PA6 range=chr4:103619161-103619277 having a high identity with TAMM41 (ENST00000273037.9) gene.
SEQ ID NO:1451 is a portion of template sequence for NFkB1 template L2a range=chr4:102505799-102505857 having a high identity with LTBP1 (ENST00000404816.6) gene.
SEQ ID NO:1452 is a portion of template sequence for NFkB1 template L2a range=chr4:102505799-102505857 having a high identity with AGBL4 (ENST00000371839.5) gene.
SEQ ID NO:1453 is a portion of template sequence for NFkB1 template L2a range=chr4:102505799-102505857 having a high identity with SMILR (NR_131202.1) gene.
SEQ ID NO:1454 is a portion of template sequence for NFkB1 template L2a range=chr4:102505799-102505857 having a high identity with EHBP1 (ENST00000405015.7) gene.
SEQ ID NO:1455-1458 are portions of template sequence for NFkB1 template AluSz6 range=chr4:102507477-102507601 having a high identity with PLCE1 (ENST00000371380.7) gene.
SEQ ID NO:1459-1465 are portions of template sequence for NFkB1 template AluSz6 range=chr4:102507477-102507601 having a high identity with KSR2 (ENST00000425217.5) gene.
SEQ ID NO:1466-1468 are portions of template sequence for NFkB1 template AluSz6 range=chr4:102507477-102507601 having a high identity with KLHL12 (NM_001303051.1) gene.
SEQ ID NO:1469 is a portion of template sequence for NFkB1 template HAL1ME range=chr4:102510807-102511027 having a high identity with DAB1 (ENST00000371236.6) gene.
SEQ ID NO:1470-1472 are portions of template sequence for NFkB1 template HAL1ME range=chr4:102510807-102511027 having a high identity with NF1 (ENST00000356175.7) and EVI2B (ENST00000330927.4) genes.
SEQ ID NO:1473 is a portion of template sequence for NFkB1 template HAL1ME range=chr4:102510807-102511027 having a high identity with CRYZL1 (ENST00000361534.6) gene.
SEQ ID NO:1474-1475 are portions of template sequence for NFkB1 template L1MA9 range=chr4:102511116-102511227 having a high identity with SLC35F3 (ENST00000366618.7) gene.
SEQ ID NO:1476-1477 are portions of template sequence for NFkB1 template LIMA9 range=chr4:102511116-102511227 having a high identity with MACF1 (ENST00000567887.5) gene.
SEQ ID NO:1478-1479 are portions of template sequence for NFkB1 template L1MA9 range=chr4:102511116-102511227 having a high identity with CTNNA3 (ENST00000433211.6) gene.
SEQ ID NO:1480-1481 are portions of template sequence for NFkB1 template L1MA9 range=chr4:102511116-102511227 having a high identity with MACF1 (ENST00000567887.5) gene.
SEQ ID NO:1482 is a portion of template sequence for NFkB1 template L2a range=chr4:102511254-102511361 having a high identity with LRP5L (ENST00000402859.6) gene.
SEQ ID NO:1483 is a portion of template sequence for NFkB1 template L2a range=chr4:102511254-102511361 having a high identity PCDH9 (ENST00000377865.6) gene.
SEQ ID NO:1484 is a portion of template sequence for NFkB1 template L2a range=chr4:102511254-102511361 having a high identity GAK (ENST00000314167.8) gene.
SEQ ID NO:1485-1491 are portions of template sequence for NFkB1 template AluJo range=chr4:102511394-102511703 having a high identity with PAUPAR (ENST00000644607.1) gene.
SEQ ID NO:1492-1497 are portions of template sequence for NFkB1 template AluJo range=chr4:102511394-102511703 having a high identity with POLR3A (ENST00000372371.7) gene.
SEQ ID NO:1498-1503 are portions of template sequence for NFkB1 template AluJo range=chr4:102511394-102511703 having a high identity with COMMD10 (ENST00000274458.8) gene.
SEQ ID NO:1504 is a portion of template sequence for NFkB1 template LIME3B range=chr4:102511709-102511897 having a high identity PPP1R16B (ENST00000299824.6) gene.
SEQ ID NO:1498-1503 are portions of template sequence for NFkB1 template AluJo range=chr4:102511394-102511703 having a high identity with COMMD10 (ENST00000274458.8) gene.
SEQ ID NO:1504 is a portion of template sequence for NFkB1 template LIME3B range=chr4:102511709-102511897 having a high identity PPP1R16B (ENST00000299824.6) gene.
SEQ ID NO:1505-1510 are portions of template sequence for NFkB1 template AluJr range=chr4:102512340-102512644 having a high identity with C SPOCK2 (NM_001244950.2) gene.
SEQ ID NO:1511-1516 are portions of template sequence for NFkB1 template AluJr range=chr4:102512340-102512644 having a high identity with TNRC6A (NM_001351850.2) gene.
SEQ ID NO:1517-1522 are portions of template sequence for NFkB1 template AluY range=chr4:102513892-102514190 having a high identity with RFX3-AS1 (ENST00000423112.2) gene.
SEQ ID NO:1523-1529 are portions of template sequence for NFkB1 template AluYa5 range=chr4:102515108-102515409 having a high identity with PLCE1 (ENST00000371380.8) gene.
SEQ ID NOS:1530-1531 are TE template sequences for NFkB1 promoter non-processive transcripts range=chr4:102499993-102500159 having high identity with RBM15 (ENST00000369784.7) gene.
SEQ ID NOS:1532 is a portion of TE template sequences for NFkB1 promoter non-processive transcripts range=chr4:102499993-102500159 having high identity with AC022634.2 (ENST00000521504.1) gene.
SEQ ID NOS:1533 is a portion of TE template sequences for NFkB1 promoter non-processive transcripts range=chr4:102499993-102500159 having high identity with RPL3 (ENST00000216146.8) gene.
SEQ ID NOS:1534 is a portion of TE template sequences for NFkB1 promoter non-processive transcripts range=chr4:102499993-102500159 having high identity with VTRNA3-1P (ENST00000362552.1) gene.
SEQ ID NOS:1535 is a portion of TE template sequences for NFkB1 promoter non-processive transcripts range=chr4:102499993-102500159 having high identity with BIRC3 (ENST00000615299.4) gene.
SEQ ID NOS:1536 is a portion of TE template sequences for NFkB1 promoter non-processive transcripts range=chr4:102499993-102500159 having high identity with InterGenic_Chr18:40901840-40901861 gene.
SEQ ID NOS:1537-1613 are TE template sequences for lncRNA_{LOC105377621}.
SEQ ID NOS:1614-1793 are TE template sequences for NFkB2.
SEQ ID NOS:1794-1888 are TE template sequences for RELA.
SEQ ID NOS:1889-2237 are TE template sequences for 1ncRNA_{RELA-DT}.
SEQ ID NOS:2238-2601 are TE template sequences for MyoD1.
SEQ ID NOS:2602-2852 are TE template sequences for lncRNA_{MyoD1}.
SEQ ID NOS:2853-3243 are TE template sequences for lncRNA_{SRA1}.
SEQ ID NOS:3244-3255 are TE template sequences for CUX2.
SEQ ID NOS:3256-3263 are TE template sequences for PRKN.
SEQ ID NOS:3264-3285 are TE template sequences for KSR2.
SEQ ID NOS:3286-3311 are TE template sequences for FAK.
SEQ ID NOS:3312-3401 are TE template sequences for Wnt2.
SEQ ID NOS:3402-3481 are TE template sequences for Wnt10B.
SEQ ID NOS:3482-3492 are TE template sequences for Wnt3A.
SEQ ID NOS:3493-3516 are TE template sequences for Wnt5B.
SEQ ID NOS:3517-3532 are TE template sequences for Wnt5A.
SEQ ID NOS:3533-3754 are TE template sequences for CRHR2.
SEQ ID NOS:3755-3767 are TE template sequences for PPARG.
SEQ ID NOS:3768-3836 are TE template sequences for NR3C1.
SEQ ID NOS:3837-3884 are TE template sequences for BRD4.
SEQ ID NOS:3885-3918 are TE template sequences for CD4.
SEQ ID NOS:3919-4076 are template DNA sequences.

### DETAILED DESCRIPTION OF THE INVENTION

### I. Definitions

“TE” refers to Transposable Elements (a.k.a. Transposons).

“TE remnant” (TEr) refers to TE no longer capable of transposition.

“Sibling TEr” refers to progeny TE that are replicated during a single transposition event that retain the sequence variations of the parent TE.

“Pathway Hub Gene” and “Index Gene” both refer to an essential gene within a biological process that is densely interconnected with other genes participating in that process; “hub” genes mediate interactions between less connected genes, therefore keeping the network together.

“Index TEr” refers to the TEr chosen from the index gene-of-interest.

“Nonprocessive transcript” (NPtx) as used herein refers to nascent RNA transcripts of variable lengths resulting from aborted transcriptional elongation of RNA-polymerases (in sense or antisense) within gene regulatory regions; wherein RNA Polymerase I, II or III initiates transcription, aborts and recycles, resulting in synthesis incomplete RNA transcripts. Euchromatin genes produce promoter and promoter-proximal nonprocessive transcripts of no known function.

“Processive transcription” refers to continuous RNA polymerase I, II or II elongation to completion of the full messenger RNA transcripts.

“Transcriptional regulatory regions” includes enhancer, promoter, promoter-proximal and intronic regions of genes.

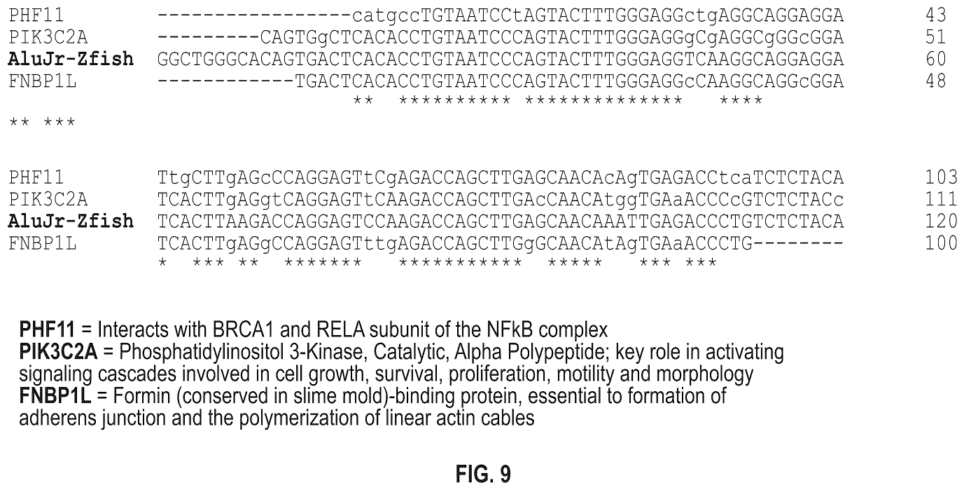

“Core Template Sequences” refers to the high identity (but not necessarily identical “sibling TE”) sequences within index TEr-aligned genes (FIG. 9). The patent claims these sequences as well as index TEr sequences.

### II. Introduction

It is of considerable importance to screen for—and treat—persons with pathogenic gene transcriptional networks such as cancer, or diseases in which multiple genes are abnormally regulated but the encoded proteins are normal, as with Parkinson's disease. The present invention fills these and other needs. The present disclosure provides for the first time that DNA sequences encoding transcripts of unknown function such as Transposable Element remnant (TEr) RNA or promoter non-processive transcripts (NPtx) have a high probability of grouping functionally-linked genes into precise pathways in silico, based on high identity nucleic acid sequence homology alone. For example, using UCSC BLAT or NCI BLASTn alignment algorithms, different TEr sequences within NFkB1 (critical cell activation gene) intron 1 were found to have a high likelihood of aligning to genes initiating epithelial to mesenchymal transition (EMT). Sharing high identity “junk” sequence occurred within transcriptional regulatory regions of functionally-linked genes of myogenesis, stress-related fat metabolism and Tₕ-immune cell activation, suggesting that protein-to-protein networks are mirrored by direct “junk-to-junk” networking between the genes that encode them. NFkB1 promoter non-processive “junk” transcripts aligned to genes participating in formation, processing, packaging and function of mRNA. The lncRNA SRA1 (Steroid Receptor RNA Activator 1) initiates transcription at a TEr that aligned multiple genes associated with Parkinson's Disease (PD), suggesting a new model of PD pathogenesis based on aberrant transcriptional network signaling, rather than malfunction of a single gene or protein.

Astonishingly, exonic TEr ofNFkB1's cis lncRNA-RP11-499E18.1 aligned some of the same EMT genes as NFkB1 intron 1 TEr, with equally high identity. SiRNA-mediated knock down of RP11-499E18.1 isoforms (546-673nt; TEr comprise 3 of 3, or 3 of 4, exons) revealed it participates in the maintenance of cell differentiation. In its absence, well-differentiated pancreatic adenocarcinoma epithelioid cells transitioned toward a mesenchymal phenotype, and poorly-differentiated pancreatic adenocarcinoma cells completely de-differentiated. The most parsimonious hypothesis for mechanism of action is that shared high identity junk RNA, dispersed by transposition over millennia and evolutionarily conserved if beneficial, contributes to the guidance of epigenetic chromatin-modifying complexes between functionally-linked genes.

Nucleic acid sequences that are shared in high identity are known to guide primed Argonautes and lncRNA to complementary sequence within the nucleus. (Xie M, Hong C, Zhang B, Lowdon R F, Xing X, Li D, et al. DNA hypomethylation within specific transposable element families associates with tissue-specific enhancer landscape. Nature Genetics. 2013; Rajan K S, Velmurugan G, Gopal P, Ramprasath T, Babu D D V, Krithika S, et al. Abundant and Altered Expression of PIWI-Interacting RNAs during Cardiac Hypertrophy. Heart Lung and Circulation. 2016; Kapusta A, Kronenberg Z, Lynch V J, Zhuo X, Ramsay L A, Bourque G, et al. Transposable Elements Are Major Contributors to the Origin, Diversification, and Regulation of Vertebrate Long Noncoding RNAs. PLoS Genetics. 2013; Profumo V, Forte B, Percio S, Rotundo F, Doldi V, Ferrari E, et al. LEADeR role of miR-205 host gene as long noncoding RNA in prostate basal cell differentiation. Nature Communications. 2019; 10(1):307; Rajasethupathy P, Antonov I, Sheridan R, Frey S, Sander C, Tuschl T, et al. A role for neuronal piRNAs in the epigenetic control of memory-related synaptic plasticity. Cell. 2012; Zhang X-O, Gingeras T R, Weng Z. Genome-wide analysis of polymerase III-transcribed Alu elements suggests cell-type-specific enhancer function. Genome research. 2019; 29(9):1402-14.)

The present inventor hypothesized that ability of transposons to disperse small groups of high-identity TE variants (TEr) during transposition, and mechanisms by which chromatin-modifiers are shuttled between genes guided by sequences of high identity complementarity suggested that high-identity TE variant sequences can themselves be signals that participate in precise gene-to-gene transcriptional crosstalk, unrelated to their subtype classification or transcription factor binding sites. Because high identity TE “siblings” (FIG. 1) disperse copies of parental TE containing small sequence variations, the potential exists that they participate in transcriptional “crosstalk” that is evolutionarily beneficial. The inventor further hypothesize that DNA “promoter slippage” nonprocessive transcripts (NPtx) are conserved following gene duplications if they are similarly beneficial.

Both TEr and NPtx sequences within key pathway genes have the potential to signal transcription rates to others within the pathway, by allowing, for example, network hub genes to communicate epigenetic transcriptional instructions to their functionally-linked partners.

The most parsimonious mechanisms by which shared high identity variant sequences contribute to transcriptional networks are:

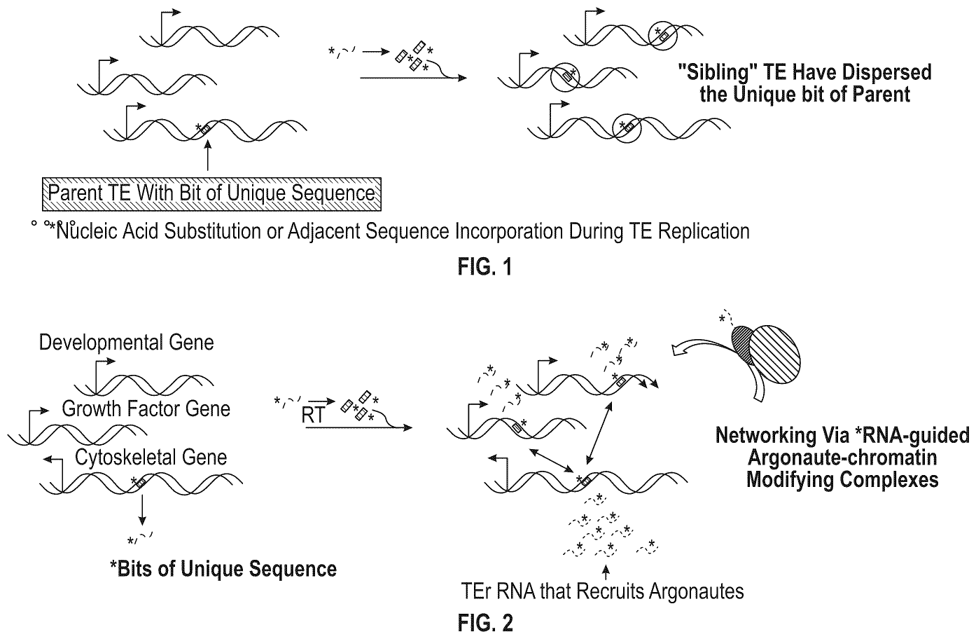

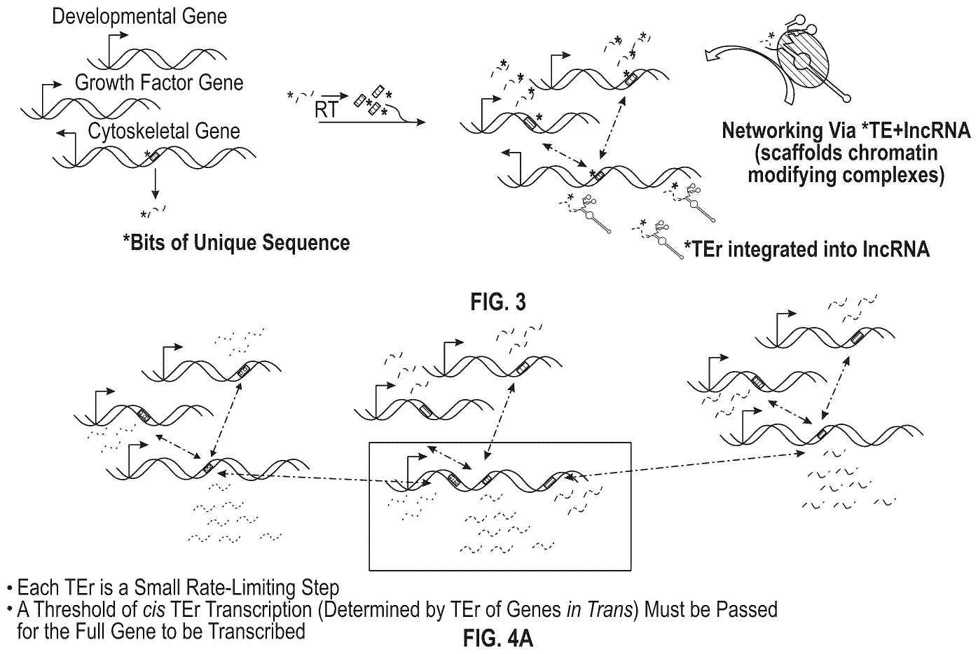

1) TEr, NPtx and other “junk” non-processive RNA transcripts become guides for “junk”-primed nuclear Argonautes (FIG. 2); and 2) nuclear lncRNA that contains exonic TEr or NPtx sequences is guided to specific DNA loci transcribing complementary sequences (FIG. 3).

Consequently, the inventor, for the first time, demonstrated that NPtx and TEr sequences of unknown function group functionally-linked genes into precise pathways, based on high identity nucleic acid sequence homology alone. These results suggest for the first time that protein networks are mirrored in the genes that encode them through the sharing of high identity “junk” DNA sequences.

The findings provide a novel method to identify nucleic acid sequences that can modulate gene-to-gene transcriptional signaling and the potential for their use (individually or in a “cocktail”) to augment, alter, block or otherwise modify the transcription of multiple genes within a network.

Accordingly, oligonucleotides (Oligos) and/or short and/or long noncoding RNAs (lncRNAs) and/or dsRNAs that function as, or are processed into, transcription activating (a)RNAs or small inhibiting (si)RNAs that are templated on the novel discovery of TEr and/or NPtx sequences that target many genes of a cellular pathway specifically and simultaneously. The invention includes modifications of the oligos such as to allow the synthetic addition of nuclear localization signals and/or “bar codes” and/or other nucleic acid identifiers and/or other synthetic modifiers.

Unlike siRNA and miRNA-mediated networks which co-regulate the cytoplasmic levels of mRNAs via complementary 3′UTR “seed” sequences, the TEr and NPtx sequences that have been identified are within gene enhancer, promoter and intronic regions. Unlike miRNA, they share high identity with other NPtx/TEr DNA in similar regions of functionally-linked genes, rather than the 3′UTR of mRNA.

Unlike piRNAs, which are specific to germ cells, TEr are expressed in somatic cells. In addition, piRNA/PIWIs primary function is thought to be the repression of actively transposing TE that could cause genetic mutation. In contrast, TEr expression may be a normal transcription regulatory activity and that TEr-primed nuclear argonautes may activate as well as suppress (return to quiescence) specific gene pathways within a somatic cell.

Unlike eRNAs, NPtx and TEr fragments are transcribed from many transcriptional regulatory regions, not just enhancer regions. To date, there are no reports of TEr sequences that have been termed “eRNA”.

Alignments were not pericentromeric and rarely in 3′UTR of coding-genes. All TE families and subtypes were represented in percentages consistent with their reported frequency in the human genome.

Unlike the multiple previous reports of TE that have been exacted to function as cell-type specific enhancers for their nearby protein-coding genes, the TEr identified here are networking between multiple genes using a mechanism other than potentially shared Transcription Factor DNA binding sites. The most parsimonious mechanism by which TEr may be networking is via RNA-mediated transcriptional gene silencing or activation.

### III. Beneficial Embodiments

1. Oligos designed with the ability to disrupt or augment a pathway, for example: activation of angiogenesis pathways might be desired in ischemic cardiac tissue whereas inhibition of angiogenesis pathway might be desired for tumor therapy.

2. There are many ways to trigger tumorigenesis and there are many different tumor types; however, common pathways are triggered when tumors progress. Oligos can be designed to inhibit common EMT pathways, thus maintaining tumor heterogeneity and responsiveness to individualized tumor therapies.

3. Alternate pathways to cell proliferation and survival can develop that lead to resistance to therapeutic interventions. For chemoresistance in tumor cells, Oligo design would target genes that initiate several pathways, including cell activation and epithelial to mesenchymal transition, templated on TEr of the NFkB1 gene.

4. Oligos designed for diagnostic and prognostic significance of diseases associated with the dysregulation of multiple genes, such as determination of levels of the single TEr sequence discovered in studies to be presented here to be associated with Parkinson's Disease.

5. Oligos designed to trigger or modify stem cells to differentiate into a tissue and/or cell type-of-interest and/or inducing specific differentiation or developmental stages in cells, tissue and/or tissue samples.

### IV. Brief Summary of Invention

The invention involves the use of novel nucleic acid sequences to detect, modulate, ablate, inhibit or augment the transcription and therefore translation and expression of functionally-linked genes.

Therapeutic nucleic acid molecules have been developed that target single genes or mRNAs are termed miRNA. Although single miRNAs can target multiple mRNAs simultaneously, miRNAs function at the posttranscriptional level, when an abnormal gene communication pathway has already begun. There is a need for molecules such as TEr and NPtx that can target multiple genes within a pathological pathway at the transcriptional level (where gene expression initiates) including genes sharing high identity TEr sequence that are otherwise unknown to be participating in the pathway.

Although the present invention has been described in considerable detail with reference to certain preferred embodiments, other embodiments are possible. The steps disclosed for a presently disclosed method, for example, are not intended to be limiting nor are they intended to indicate that each step is necessarily essential to the method, but instead are exemplary steps only. Therefore, the scope of the appended claims should not be limited to the description of preferred embodiments contained in this disclosure.

### V. Embodiments

In a first set of embodiments, the invention provides the method of identifying DNA sequences that are shared by several genes participating in an individual biologic pathway.

In a second set of embodiments, the invention provides methods of determining nucleic acid template sequences against which gene activating or inhibitory molecules can be designed and directed, including, but not restricted to, small interfering RNAs (siRNA), short hairpin RNA (shRNA), morpholino, or antisense oligonucleotides; for diagnostic, prognostic or therapeutic purposes.

In the first and second set of embodiments, the sequence is a transposon that is an autonomous element or a nonautonomous element. The transposon can also be a DNA transposon or a retrotransposon, including an LTR retrotransposon and a non-LTR retrotransposon. More specifically, an LTR retrotransposon can include an endogenous retrovirus (ERV); and a non-LTR retrotransposon can include a SINE retrotransposon, such as an Alu sequence or SINE-VNTR-Alus (SVA); or a LINE element, such as L1, or a LINE-like element, such as R1 or R2.

In the first and second set of embodiments, the sequence is the product of non-processive transcription within a gene promoter, its 5′ or 3′ enhancer (sequence not otherwise claimed as “enhancer RNA” or “lncRNA”) or the transcriptional regulatory region of an intron.

In a third set of embodiments, the invention provides methods of delaying Epithelial to Mesenchymal Transition and/or cancer stem cell proliferation, comprising administering to a subject in need of such treatment an effective amount of TE sequence complementary to expressed pathway-specific TE or NPtx.

In a fourth set of embodiments, the invention provides methods of delaying pathologic cardiovascular decline, or stimulation of myoblast/myocyte regeneration following ischemic or other insult, comprising administering to a subject in need of such treatment an effective amount of TE sequence complementary to expressed pathway-specific TE or NPtx.

In a fifth set of embodiments, the invention provides methods of diagnosing and delaying pathologic neuronal decline, comprising administering to a subject in need of such treatment an effective amount of TE sequence complementary to expressed pathway-specific TE or NPtx.

In a sixth set of embodiments, the invention provides methods of modulating pathologic abnormalities of any and all cellular or tissue pathways, comprising administering to a subject in need of such treatment an effective amount of TE sequence complementary to expressed pathway-specific TE or NPtx.

In a seventh set of embodiments, the invention provides methods of activating latent viral and/or “hidden” quiescent metastatic cells, such that therapy targeting actively proliferating virus or cells can be implemented.

In other embodiments, the invention provides methods to trigger or modify stem cells to differentiate into a tissue and/or cell type-of-interest and/or inducing specific differentiation or developmental stages in cells, tissue and/or tissue samples.

In other embodiments, the invention provides recombinant nucleic acid sequences for detection and monitoring of diseases including, but not restricted to, autoimmune disease, cardiovascular disease, metabolic syndrome, obesity, neurodegenerative disease, and proliferative or oncogenic diseases.

In other embodiments, the invention provides recombinant nucleic acid sequences for detection and analysis of potentially active or inactive pathways in vitro.

In another aspect of the methods, the NPtx and TE-template oligonucleotide is a mixture, or a “cocktail” formulated as a pharmaceutical composition and is administered to the subject in a therapeutically effective amount. The oligonucleotide may also be administered together or in conjunction with other agents.

The present invention also includes additions or modification to nucleic acid sequences claimed here that directs its nuclear import.

The present invention also includes a cell comprising any of recombinant nucleic acid sequences designed using the Method. The invention also includes a transgenic animal, including a transgenic vertebrate, comprising any of the recombinant nucleic sequences designed using the Method (or cell that contains any of them).

In one or more embodiments, the present invention includes a synthetic nucleic acid comprising one or more of a transposon remnant, a promoter and/or a promoter-proximal non-processive transcript, and selected to modulate gene-to-gene transcriptional signaling within a given functional pathway. In some embodiments, the synthetic nucleic acid to further modulate transcription of a plurality of genes within a network.

In some embodiments, the synthetic nucleic acid has a sequence that aligns with high identity to transcriptional regulatory regions of genes participating in the given functional pathway. The high identity is defined based on UCSC BLAT and/or NCBI BLASTn alignment or other quality controlled alignment algorithm.

In some embodiments, the synthetic nucleic acid has a sequence selected from top ten BLAT₂₀₁₃ alignments.

In some embodiments, the synthetic nucleic acid—also includes nuclear localization sequences.

In some embodiments, the given functional pathway is selected from the group consisting of epithelial to mesenchymal transition pathway, phospholipid signaling pathway, myogenesis pathway, stress-mediated fat metabolism pathway, CD4+ T-cell activation and HIV binding pathway, and a Parkinson's Disease-associated pathway.

In one or more embodiments, the present invention includes a method of modulating epigenetic communication between genes coordinating specific pathways. The method includes delivering one or more of the synthetic nucleic acids disclosed herein to a sample of cells and/or a tissue.

In some embodiments, delivering the one or more synthetic nucleic acids comprises a delivery vehicle comprising the one or more nucleic acids, and nanoparticles or extracellular vesicles.

In some embodiments, modulating the epigenetic communication between genes coordinating specific pathways comprises ablate, inhibit or augment the transcription, translation or expression of one or more of functionally-linked genes.

In some embodiments, the method further includes determining a set of functionally-linked genes. In some embodiments, determining the set of functionally-linked genes comprises: (a) selecting a transposon remnant, a promoter, or a promoter-proximal non-processive transcript of a first index gene from a given functional pathway; (b) identifying, using a computer implemented sequence alignment algorithm implemented by a processor, transposon remnant sequences from a set of genes, having at least 75% homology with the selected transposon remnant, promoter, or promoter-proximal non-processive transcript; (c) determining, by the processor, a genomic position of the transposon remnant sequences with highest sequence identity with the selected transposon remnant, promoter, or promoter-proximal non-processive transcript; (d) in response to a determination that the genomic position of a given identified transposon remnant sequence is within a gene regulatory region of a first gene among the set of genes, tabulating, by the processor, function of the first gene; (e) repeating (a)-(d) for identified transposon remnant sequences that are in cis to the selected transposon remnant, promoter, or promoter-proximal non-processive transcript to determine transposon remnant sequences of genes connected to the first index gene; and (f) repeating ((e) with transposon remnant sequences of genes, among the set of genes, connected to the first index gene to determine a group of genes forming the given functional pathway.

In some embodiments, the method further includes: (g) repeating (a)-(f) for a second index gene.

In one or more embodiments, the invention includes a method of determining a network of genes, the method comprising the steps of (a) selecting a transposon remnant, a promoter, or a promoter-proximal non-processive transcript of a first index gene from a given functional pathway; (b) identifying, using a computer implemented sequence alignment algorithm implemented by a processor, transposon remnant sequences from a set of genes, having at least 75% homology with the selected transposon remnant, promoter, or promoter-proximal non-processive transcript; (c) determining, by the processor, a genomic position of the transposon remnant sequences with highest sequence identity with the selected transposon remnant, promoter, or promoter-proximal non-processive transcript; (d) in response to a determination that the genomic position of a given identified transposon remnant sequence is within a gene regulatory region of a first gene among the set of genes, tabulating, by the processor, function of the first gene; (e) repeating (a)-(d) for identified transposon remnant sequences that are in cis to the selected transposon remnant, promoter, or promoter-proximal non-processive transcript to determine transposon remnant sequences of genes connected to the first index gene; and (f) repeat (a)-(e) with transposon remnant sequences of genes, among the set of genes, connected to the first index gene to determine a group of genes forming the given functional pathway.

In some embodiments, the method may further include: (g) repeating (a)-(f) for a second index gene. In some embodiments, in response to a determination that the group of genes determined for the second index gene is different from the group of genes for the first index gene, determining that second index gene is from a functional pathway different from that of the given functional pathway.

In some embodiments, the selected transposon remnant, promoter, or promoter-proximal non-processive transcript includes one or more of a from one or more of a transcribed transposon remnant, an ancient transposon remnant, a conserved transposon remnant, a promoter region that is separated from a transcription start site by less than 5 kilobases (kb), an enhancer region that is separated from a promoter by less than 50 kb, promoter-proximal region, 5′ untranslated region; 3′ untranslated region, a first intron proximal to a transcription start site, and a non-processive transcript region in regulator region or a first intron proximal to a promoter.

In some embodiments, the first index gene is selected from 2013 UCSC human genome database.

In some embodiments, the computer implemented sequence alignment algorithm is BLAT₂₀₁₃.

In some embodiments, the given functional pathway is selected from the group consisting of epithelial to mesenchymal transition pathway, phospholipid signaling pathway, myogenesis pathway, stress-mediated fat metabolism pathway, CD4+ T-cell activation and HIV binding pathway, and a Parkinson's Disease-associated pathway.

In some embodiments, identifying transposon remnant sequences from a set of genes comprises identifying transposon remnant sequences having at least 90% homology with the selected transposon remnant, promoter, or promoter-proximal non-processive transcript.

In one or more embodiments, the present invention may include a method for inducing specific differentiation or developmental stages in cells. The method may include determining a group of genes forming a given functional pathway using a method of described herein; and delivering one or more synthetic nucleic acids comprising one or more of a transposon remnant, a promoter and/or a promoter-proximal non-processive transcript, and selected to modulate gene-to-gene transcriptional signaling within the given functional pathway. The given functional pathway is associated with the specific differentiation or developmental stages in cells.

In some embodiments, the one or more synthetic nucleic acids have a sequence that aligns with high identity to transcriptional regulatory regions of genes participating in the given functional pathway. In some embodiments, high identity is defined based on BLAT₂₀₁₃ alignment. In some embodiments, the synthetic nucleic acid has a sequence selected from top ten BLAT₂₀₁₃ alignments.

In some embodiments, the one or more synthetic nucleic acids further include nuclear localization sequences.

In some embodiments, delivering the one or more synthetic nucleic acids comprises delivering a delivery vehicle comprising the one or more nucleic acids, and nanoparticles or extracellular vesicles.

In some embodiments, the method may further include modulating the epigenetic communication between the group of genes forming the given functional pathway.

In some embodiments, modulating the epigenetic communication comprises one or more of ablating, inhibiting or augmenting the transcription, translation or expression of one or more of functionally-linked genes.

In some embodiments, the method may further include delivering an oligonucleotide selected to ablate, inhibit or augment the transcription, translation or expression of one or more of functionally-linked genes.

More generally, the invention is further directed to the general and specific embodiments defined, respectively, by the independent and dependent claims appended hereto, which are incorporated by reference herein.

### VI. Summary of TE Subtypes

TE subtypes are described in detail in Wells and Feschotte (Wells J N, Feschotte C. A Field Guide to Eukaryotic Transposable Elements. Annu Rev Genet. 2020; 54:539-61). In brief, DNA transposons use a “cut-and-paste” mechanism of replication. TEs that replicate via an RNA intermediate (“copy-and-paste”) include Long Interspersed Elements (LINEs), Short INterspersed elements (SINEs) and Long Terminal Repeat (LTR) retrotransposons. DNA, LTR and LINE elements contain RNA Pol2 binding sites and SINEs contain RNA Pol3 binding sites. SINEs, including the most numerous in the human genome, Alu Repeats, co-opt the LINE replication machinery to transpose. Mammalian-wide interspersed repeats (MIRs, the most ancient family of TEs in the human genome at >550 million years old; a.k.a “fossils”) are core sequences of tRNA-derived SINEs.

### EXAMPLES

### Example 1

Embodiments presented herein are based on the unique finding that Transposable Element remnant (TEr) RNA or promoter non-processive transcripts (NPtx) have a high probability of aligning with high identity to transcriptional regulatory regions of functionally-linked genes, suggesting that they participate in beneficial transcriptional crosstalk. In vitro data supports a functional requirement for “junk” sequences chosen from the key cell activation gene NFkB1. This in silico pattern occurred in multiple pathway-specific genes, including genes coordinating phospholipid signaling-mediated cell activation, epithelial to mesenchymal transition (EMT), myogenesis, stress-related fat metabolism and Tₕ-immune cell activation. A single TEr was shared with high identity between genes associated with Parkinson's Disease. In vitro analysis of TEr ofNFkB1cis lncRNA, which aligned with high identity to some of the same genes of EMT initiation as NFkB1 intron 1 TEr, revealed their participation in the maintenance of cell differentiation in cancer cells, as had been predicted by the in silico method disclosed herein.

The sequences disclosed herein are different than TE subtype-specific sequence or “similar control regions” such as shared transcription factor DNA binding sites. These NPtx and TEr sequences have not otherwise been classified as miRNA, piRNA, siRNA, eRNA or other RNA of known function. The invention includes nucleic acid sequences predicted to detect, modulate, ablate, inhibit or augment the transcription of genes of the above listed pathways.

The ability of transposition to disperse small groups of high-identity TE variants (“siblings”, FIG. 1) suggested the hypothesis that TEr participate in precise gene-to-gene transcriptional crosstalk based on shared nucleic acid sequences of high identity, unrelated to their transcription factor DNA binding sites or TE subtype-specific RNA secondary structure. High identity nucleic acid sequences guide Argonaute/chromatin-modifying complexes to nascent nuclear RNA containing complementary sequences (FIG. 2), as well as guide lncRNA-transcription factor scaffolds to specific genomic loci (FIG. 3); TEr have been shown to participate in both mechanisms of transcriptional regulation in somatic tissue. (Xie M, Hong C, Zhang B, Lowdon R F, Xing X, Li D, et al. DNA hypomethylation within specific transposable element families associates with tissue-specific enhancer landscape. Nature Genetics. 2013; Chishima T, Iwakiri J, Hamada M. Identification of transposable elements contributing to tissue-specific expression of long non-coding RNAs. Genes. 2018; Rajan K S, Velmurugan G, Gopal P, Ramprasath T, Babu D D V, Krithika S, et al. Abundant and Altered Expression of PIWI-Interacting RNAs during Cardiac Hypertrophy. Heart Lung and Circulation. 2016; Kapusta A, Kronenberg Z, Lynch V J, Zhuo X, Ramsay L A, Bourque G, et al. Transposable Elements Are Major Contributors to the Origin, Diversification, and Regulation of Vertebrate Long Noncoding RNAs. PLoS Genetics. 2013; Profumo V, Forte B, Percio S, Rotundo F, Doldi V, Ferrari E, et al. LEADeR role of miR-205 host gene as long noncoding RNA in prostate basal cell differentiation. Nature Communications. 2019; 10(1):307; Rajasethupathy P, Antonov I, Sheridan R, Frey S, Sander C, Tuschl T, et al. A role for neuronal piRNAs in the epigenetic control of memory-related synaptic plasticity. Cell. 2012; Holdt L M, Hoffmann S, Sass K, Langenberger D, Scholz M, Krohn K, et al. Alu Elements in ANRIL Non-Coding RNA at Chromosome 9p21 Modulate Atherogenic Cell Functions through Trans-Regulation of Gene Networks. PLoS Genetics. 2013; Alfeghaly C, Sanchez A, Rouget R, Thuillier Q, Igel-Bourguignon V, Marchand V, et al. Implication of repeat insertion domains in the trans-activity of the long non-coding RNA ANRIL. Nucleic Acids Research. 2021; 49(9):4954-70; KD, Ameen M, Guo H, Abilez O J, Tian L, Mumbach M R, et al. Endogenous Retrovirus-Derived lncRNA BANCR Promotes Cardiomyocyte Migration in Humans and Non-human Primates. Dev Cell. 2020; 54(6):694-709.e9; La Greca A, Scarafia M A, Hernández Cañás M C, Pérez N, Castañeda S, Colli C, et al. PIWI-interacting RNAs are differentially expressed during cardiac differentiation of human pluripotent stem cells. PLoS One. 2020; 15(5):e0232715.)

With the hypothesis that TEr variant sequences participate in RNA-mediated gene-to-gene transcriptional crosstalk that is evolutionarily beneficial, we tested the common assumption that “junk” variant TEr are physiologically irrelevant. Taking advantage of the sequence variations within individual TEr that allows their precise genomic positioning by computer algorithm, we examined the rate at which TEr sequences align in silico with high identity to other genes, and the position and identity of the genes to which they aligned (EXAMPLE 2). TEr were chosen from enhancer, promoter and intronic (predominantly promoter-proximal intron 1) regions of genes critical to three biologic pathways (“hub” genes). In a larger bioinformatics study, the rate of TEr alignments to pathway-specific genes within a biological pathway was contrasted to the rate of TEr alignments to pathway-specific genes of the other two groups (EXAMPLE 3). In addition, complete sets of enhancer, promoter and intron 1 TEr were evaluated for the individual hub genes NFkB1 and MyoD1 (EXAMPLES 4 and 5). The rate of their TEr alignments to pathway-specific genes were contrasted to random TEr and those of housekeeping genes. Significant sequence genomic alignment was arbitrarily defined as the top ten BLAT₂₀₁₃ alignments of UCSC database BLAT-2013(GRCh38/hg38). (Kent W J. BLAT—The BLAST-Like Alignment Tool. Genome Research. 2002.) Because TE contain repetitive sequence, it was anticipated that TEr genomic alignments would be abundant and random.

Surprisingly, the likelihood is high that TEr sequences derived from transcriptional regulatory regions of key pathway genes will align with high identity to other genes within the same pathway (EXAMPLES 6-10). Alignment is not linked to TFBS or subtype-specific sequence. Many TEr alignments were intergenic, to lncRNA of unknown function, or to genes with function that could not be directly associated with a specific pathway. However, the probability was high that both pathway-critical hub genes and, astonishingly, their adjacent (cis) lncRNA, contained TEr with high identity to other pathway-specific genes and, not infrequently, to different regions within the same gene (EXAMPLE 4). For example, primary cell-activation gene NFkBT and its cis lncRNA_{LOC105377621/RP11-499E18.1} contain TEr sequences that aligned with high identity to the same genes critical to epithelial to mesenchymal transition (EMT), including Latent-Transforming Growth Factor Beta-Binding Protein 1 (LTBP1) and Phosphatidylinositol-4-phosphate 3-kinase (PI3K). Numerous other genes of EMT were aligned by TEr ofNFkB1 or lncRNA_{LOC105377621/RP11-499E18.1}.

In vitro data confirms the predictive value of the method disclosed herein in designing a molecule based on these sequences that is a powerful modulator of epithelial to mesenchymal transition in pancreatic adenocarcinoma cell lines (EXAMPLE 4).

Hub gene TEr within other cellular pathways were also examined for genomic alignment. This pattern of in silico alignments was repeated in other critical genes related to EMT, such as FAK/PTK, b-Catenin and Wnt isoforms (EXAMPLES 4, 8). While most TEr were only transcribed at minimal levels if at all, numerous TEr in MyoD1 (Muscle Differentiation 1) promoter/enhancer regions were strongly expressed in HSMM (skeletal myoblast) cells; these too had a high likelihood of alignment with high identity to TEr within other critical genes of myogenesis (EXAMPLE 5). Astonishingly, TEr sequences from SRA1 lncRNA (required for retinoic acid-mediated neuronal cell differentiation) aligned to numerous genes associated with Parkinson's Disease (EXAMPLE 6), suggesting a new model of disease pathogenesis in which mis-regulation of TEr transcription leads to aberrant guidance of transcription effector-complexes between the genes that share them.

Other promoter-proximal non-TEr transcripts were also analyzed for genomic alignments. Antisense nonprocessive transcripts (NPTx; termed “promoter slippage”; EXAMPLE 7) are often considered “junk”. The transcribed antisense promoter sequences of NFkB1 were analyzed. They were found to have a high probability of aligning to genes encoding RNA-binding proteins required for RNA transcription, formation and packaging, as will be demonstrated (EXAMPLE 7).

Finally, hub gene TEr were examined in the stress-response pathway gene CRHR2 (receptor for stress-related hormone CRF; EXAMPLE 9) and in inflammatory pathway gene CD4+ (T_{H} immune cell activation, HIV binding; EXAMPLE 10). Again, the probability remained high that these TEr aligned to other genes within their specific pathways, as disclosed herein.

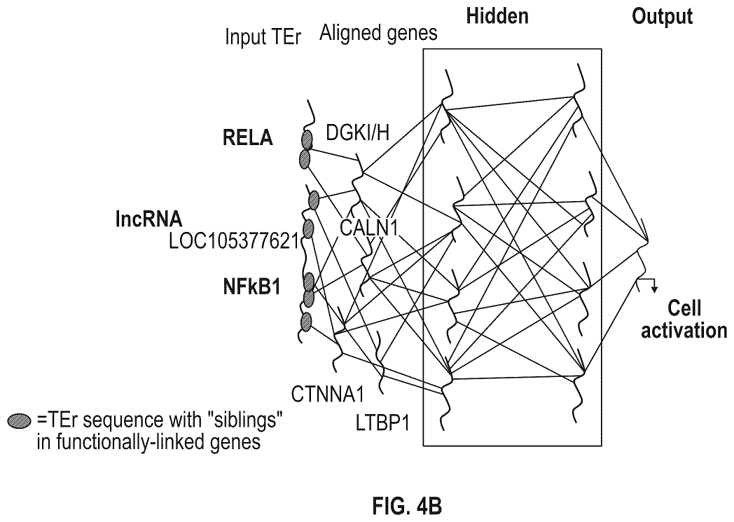

The present inventors are reporting, for the first time, that protein-to-protein interactive networks are mirrored in the genes that encode them, through the sharing of high identity variant TEr sequences. What is unique to the results presented herein is that they suggest individualized high identity remnant TEr sequences participate in beneficial transcriptional crosstalk irrespective of their subtype or “similar control regions” such as shared TFBS. Although many TEr may in fact be nonfunctional residues, these results predict that many more than the expected number of TEr provide a rate-limiting step for transcription elongation based on RNA-sequence mediated epigenetic regulation. In this model, the final transcription rate of a full-length mRNA is the summation of the rate at which each TEr is epigenetically (controlled in turn by the transcription rates of its siblings in trans) (FIG. 4a). This model of effector complexes guided between genes containing “sibling” TE predicts “neural-like” networks will naturally form (FIG. 4b).

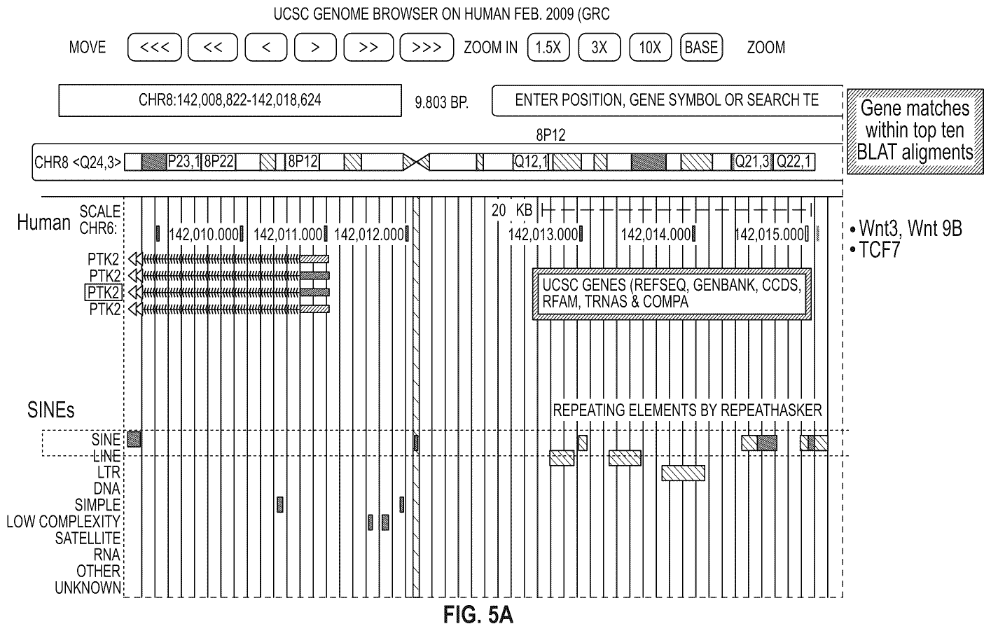

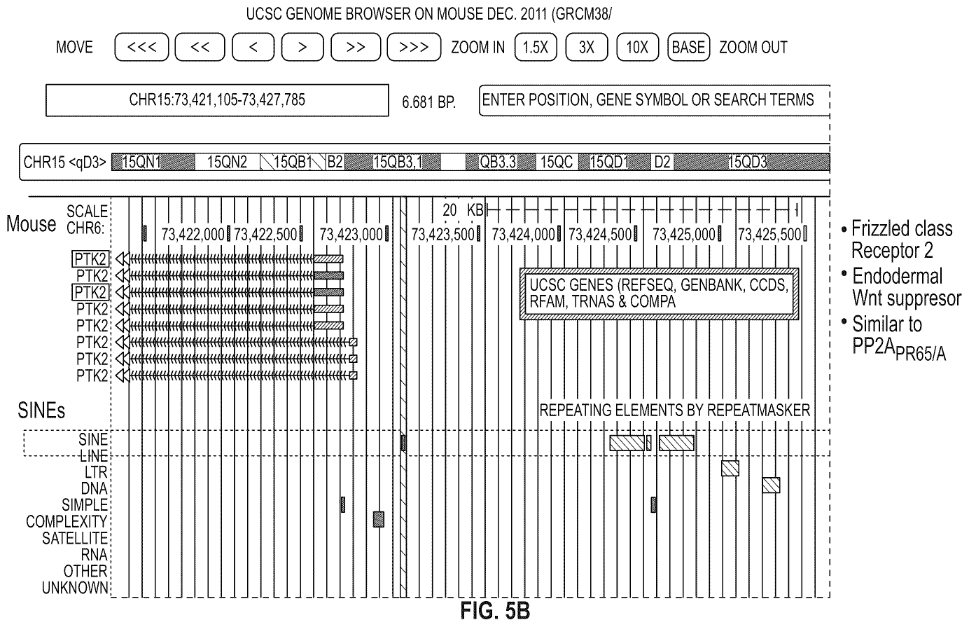

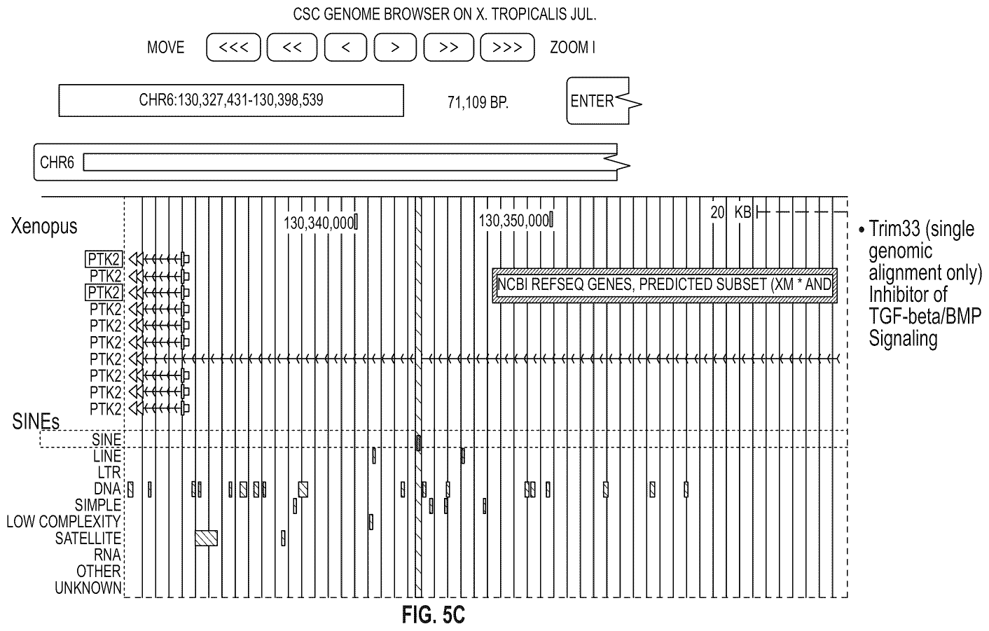

The model also sheds light on a process whereby random distribution of TE siblings could result in highly specific gene networks. If, as already described, TE siblings integrate within genes for which transcriptional crosstalk becomes evolutionarily beneficial, their sequences are conserved. Subsequent random transposition events from one of these siblings (now the “parent”, FIG. 1) are once again conserved if their integration has further allowed beneficial crosstalk with the genes already sharing the high identity sequence (alreadyfunctionally-linked). If, following species divergence, the TE transposes again, the specific genes aligned would be different between the species, but again, the sequence would only be conserved if beneficial crosstalk occurred between already functionally-linked genes. This model would explain the highly conserved MIR remnant within the promoter of FAK/PTK2 (essential role in regulating cell migration, adhesion, spreading) of Human, Xenopus and Murine species that aligned to EMT-critical genes, but to different ones: Human MIR aligned between Wnt3/Wnt9B and to TCF7 (activates transcription through Wnt/beta-catenin signaling pathway) while Murine MIR aligned to FZD2 (Frizzled class Receptor 2; a Wnt receptor) and BARX1 (an endodermal Wnt suppressor) whereas Xenopus SINE2-1/MIR aligned only once within the full genome: to TRIM33 (tripartite motif containing 33; an inhibitor of TGF-beta-mediated EMT signaling) (FIG. 5).

Transcription factors are powerful machines of gene transcription regulation. Nevertheless, it is not well-understood how genes that coordinate specific biologic pathways “find” each other for co-regulation, and how DNA accessibility and transcription remains dynamic, yet gene-specific, within generally activated or inhibited microenvironments. Evolution has been prolific in taking advantage of the principles of nucleic acid complementarity that allows precision in RNA/DNA-mediated signaling and targeting of proteins. The present disclosure is based on results that suggest complex gene-to-gene communication networks have evolved through the simple repetition of nucleic acid sequence duplication and dispersal within the genome, amplified by transposons, over millions of years.

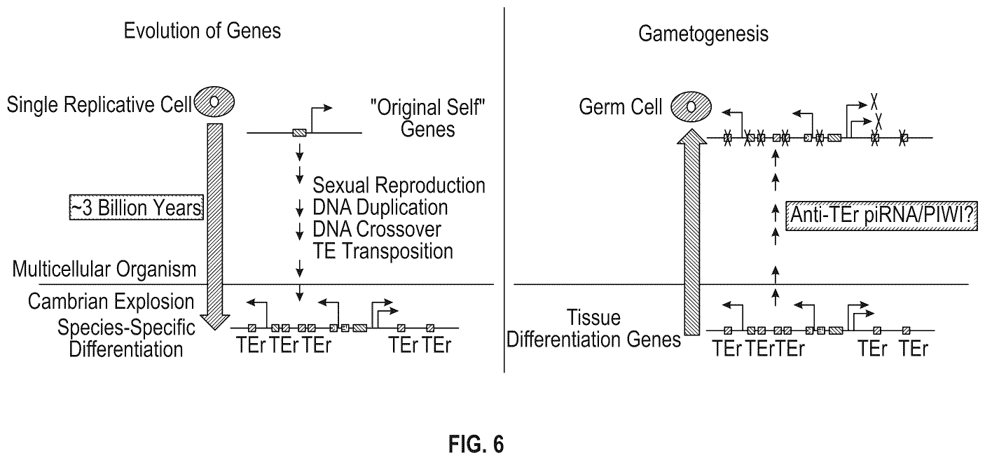

Finally, the inventors suggest that the dramatic expression and then silencing of TEr during gametogenesis and embryogenesis is not primarily an “immune-like” response “genomic parasites”. (Malone C D, Hannon G J. Small RNAs as Guardians of the Genome. 2009). PiRNA-PIWI complexes do not disturb or damage TEr sequences, they silence them temporarily. Many individual TEr are expressed in a controlled and cell-type specific way for unknown reasons. (Hall L L, Carone D M, Gomez A V, Kolpa H J, Byron M, Mehta N, et al. Stable C0T-1 repeat RNA is abundant and is associated with euchromatic interphase chromosomes. Cell. 2014; Carnevali D, Conti A, Pellegrini M, Dieci G. Whole-genome expression analysis of mammalian-wide interspersed repeat elements in human cell lines. DNA research: an international journal for rapid publication of reports on genes and genomes. 2017; Xie M, Hong C, Zhang B, Lowdon R F, Xing X, Li D, et al. DNA hypomethylation within specific transposable element families associates with tissue-specific enhancer landscape. Nature Genetics. 2013; Johnson J M, Edwards S, Shoemaker D, Schadt E E. Dark matter in the genome: Evidence of widespread transcription detected by microarray tiling experiments. 2005; Chishima T, Iwakiri J, Hamada M. Identification of transposable elements contributing to tissue-specific expression of long non-coding RNAs. Genes. 2018). Perhaps the advantages TEr have contributed to the evolution of multicellularity and tissue differentiation is conserved by piRNA/PIWI complexes, just silenced as the organism prepares to replicate—a single cell once again. (FIG. 6).

In summary, the common assumption that the small sequence variation that allows determination of the genomic position of a repetitive element is physiologically irrelevant “junk” was tested. Surprisingly, results suggest that protein-to-protein networks are mirrored by direct gene-to-gene networks between the genes that encode them, through the sharing of high identity “junk” DNA sequences. The unexpected specificity of this “junk” indicates its potential role in guidance of epigenetic chromatin-modifying complexes between functionally-linked genes by TEr-primed Argonautes and TEr-containing lncRNA. In addition, results suggest a new model of disease pathogenesis in which mis-regulation of TEr transcripts leads to aberrant guidance of transcription effector-complexes between the genes that share complementary partners, creating a transcription “network-opathy”. Results presented in this patent suggests this may be the case in certain forms of Parkinson's disease. In vitro data confirms the predictive value of the Method in designing a molecule that is a powerful modulator of epithelial to mesenchymal transition (EXAMPLE 4).

These NPtx and TEr sequences have not otherwise been classified as miRNA, piRNA, siRNA, eRNA or other RNA of known function. Shared high-identity sequences ranged in length from 20 bp to hundreds of base pairs. They were sometimes transcribed in cell-type specific patterns into small RNA fragments unrelated to transposition. They were often found in lncRNA. Alignments were not pericentromeric and rarely in 3′UTR of coding-genes. All TE families and subtypes were represented in percentages consistent with their reported frequency in the human genome.

Overall, the common assumption that the small sequence variation that allows determination of the genomic position of a repetitive element is physiologically irrelevant “junk” was tested. Surprisingly, results suggest that protein-to-protein networks are mirrored by direct gene-to-gene networks between the genes that encode them, through the sharing of high identity “junk” DNA sequences. The unexpected specificity of this “junk” indicates its potential role in guidance of epigenetic chromatin-modifying complexes between functionally-linked genes by TEr-primed Argonautes and TEr-containing lncRNA. In addition, results suggest a new model of disease pathogenesis in which mis-regulation of TEr transcripts leads to aberrant guidance of transcription effector-complexes between the genes that share complementary partners, creating a transcription “network-opathy”. Results presented in this patent suggests this may be the case in certain forms of Parkinson's disease. In vitro data confirms the predictive value of the Method in designing a molecule that is a powerful modulator of epithelial to mesenchymal transition.

These NPtx and TEr sequences have not otherwise been classified as miRNA, piRNA, siRNA, eRNA or other RNA of known function. Shared high-identity sequences ranged in length from 20 bp to hundreds of base pairs. They were sometimes transcribed in cell-type specific patterns into small RNA fragments unrelated to transposition. They were often found in lncRNA. Alignments were not pericentromeric and rarely in 3′UTR of coding-genes. All TE families and subtypes were represented in percentages consistent with their reported frequency in the human genome.

### Example 2: Identifying Gene Networks in Silico

In one example, the present invention includes a method by which gene networks are identified in silico.

In brief, the Method can be summarized as follows:

1. Choose TEr or NPtx of interest. These include, but are not limited to, those within enhancer, promoter and promoter-proximal regions; 5′UTR, 3′UTR; Intron 1 proximal to the TSS; and/or NPtx, not otherwise annotated, in all regulatory regions and introns.

2. Using a quality-controlled sequence alignment algorithm (BLAT, BLASTn), identify TEr and other high identity sequence with criteria allowing a high probability of high identity. For example, (but not restricted to): NCBI “BLASTn”-2013: Transcripts+top 15 intronic hits, E=0.0, % homology >75%; and/or UCSC Genome Browser: Duplicates >1000, Human Chain Sequence Alignments, “BLAT”-2013 top 20 hits, homology >75%.

3. Sequences of highest identity are checked for genomic position. If they are within a gene regulatory region (intronic, promoter-proximal or enhancer to a coding or noncoding gene) the full function of that gene is tabulated, to the extent that it is known.

4. The process is reiterated with TEr sequence found in cis to the original TEr.

5. The process is reiterated with TEr sequences of genes thus connected to the index gene.

6. Gene functional groups, identified by Steps 1-5, can be statistically compared to groups of genes identified using a different index gene. If the groups are significantly different, the index genes are members of different functional pathways.

METHOD in detail,
key pathway genes (Index Genes) and the TEr chosen from their transcriptional regulatory regions (Index TE) were chosen using the criteria listed in Table 1.

TABLE 1
Criteria for Index Gene and TEr selection
KEY PATHWAY GENE (INDEX GENE)
Critical to pathway of interest
“Hub” protein in signal transmission
Conserved
TEr SEQUENCES CHOSEN (INDEX TE)
Gene transcriptional regulatory regions
Transcribed
Conserved
Transcription Start Site (TSS) proximal
5′UTR
Promoter proximal intron 1
Adjacent to TEr of interest

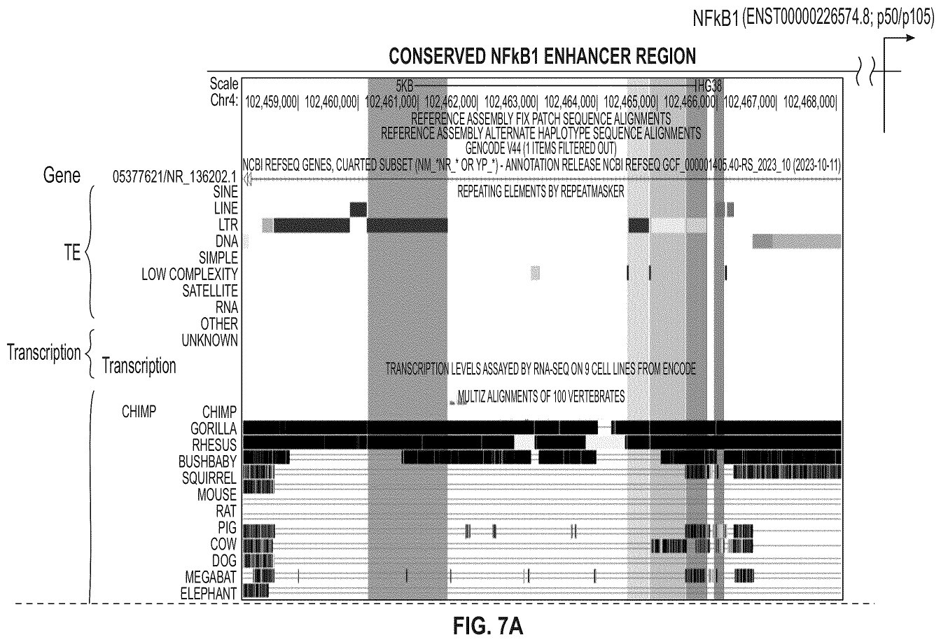

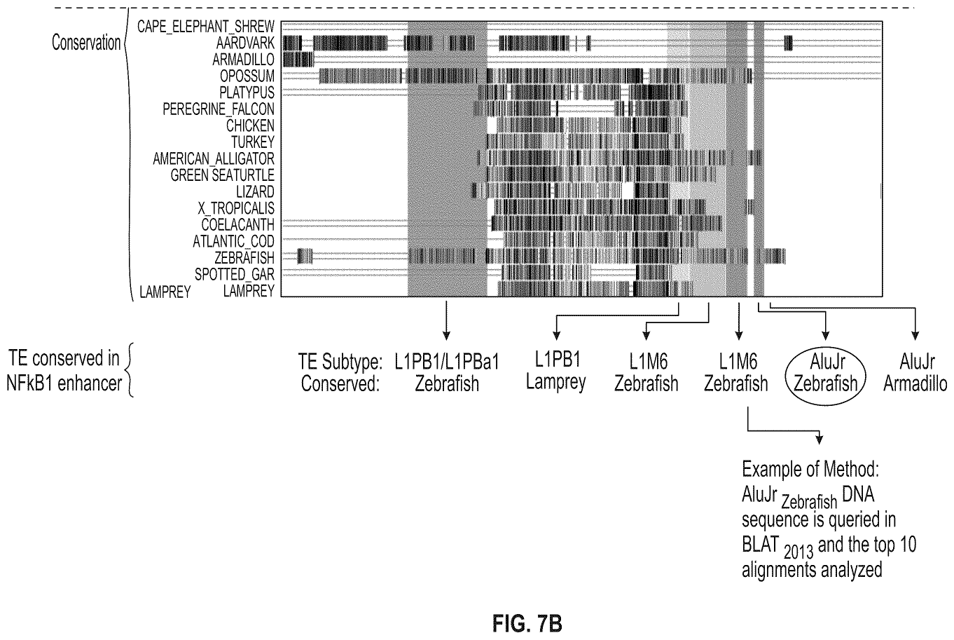

For each Index Gene chosen, attention was focused initially on transcribed TEr, highly conserved TEr and their adjacent TEr (TE subtypes are described in detail elsewhere herein) (exemplified in FIG. 7). For Index Genes NFkB1 and MyoD1, TEr integrated within all transcriptional regulatory regions were analyzed including promoter (defined as up to 5kb from the transcription start site), enhancer (within 50kb of the promoter) and promoter-proximal Intron 1.

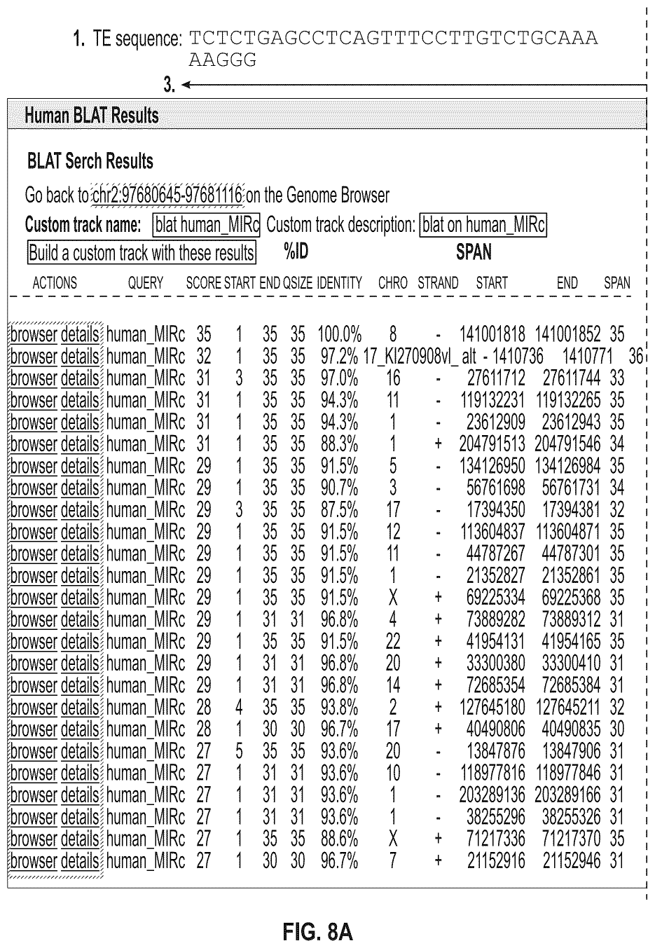

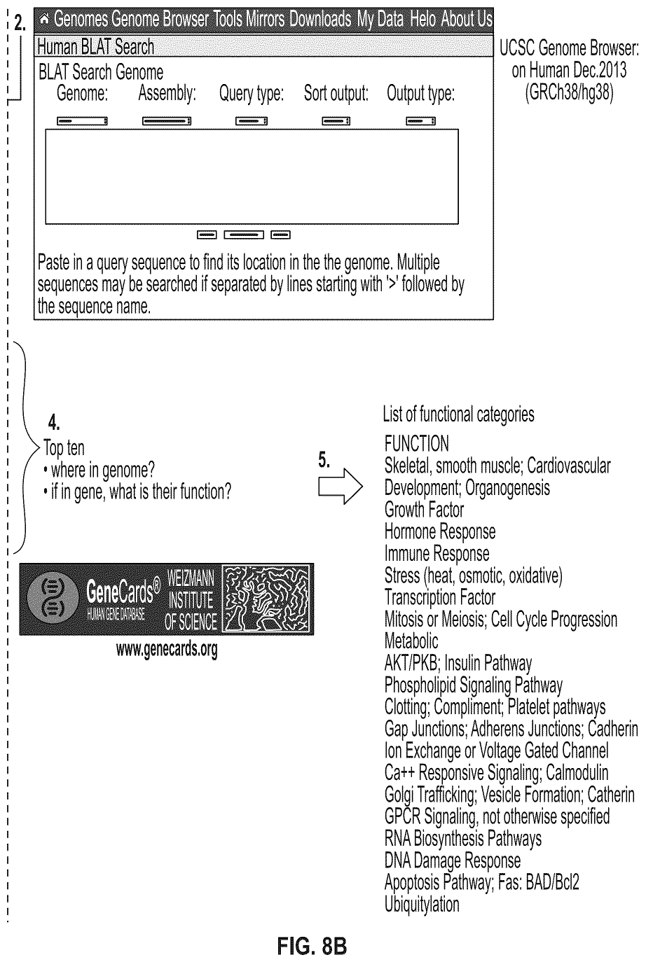

Using a quality-controlled sequence alignment algorithm, TEr alignments with the highest probability of high identity (as defined and ranked by the alignment algorithm of choice) are determined (FIG. 8). For example, (not the only possible criteria):

NCBI “BLASTn”: Transcripts+top intronic hits, chance the alignment is random (E)=significant=% homology>75%.

UCSC genome database BLAT2013 (GRCh38/hg38)(“BLAT2013”): top 10 alignments were chosen for experiments reported in this patent (exemplified in Table 2). BLAT on DNA is designed to find sequences of ≥95% similarity of length 25 bases or more, and perfect sequence matches of 20 bases (Kent W J. BLAT—The BLAST-Like Alignment Tool. Genome Research. 2002.) (FIG. 9; These aligned sequences are TEr “siblings” (as defined FIG. 1). Those claimed in this patent are termed “Core Template Sequences”.

TABLE 2
Example of top 10 BLAT₂₀₁₃ alignments of NFkB1 TEr sequence of
AluJr_{Zebrafish} of FIG. 7)

| | | | | Summary | |
|---|---|---|---|---|---|
| # | Location | Conservatn | Txn | Description | Gene |
| 1 | alignment | Zebrafish | — | | |
| | to self | | | | |
| 2 | ln 3/9 | Arm | +/−all | PHF11 Interacts with | PHF11 (ENST00000378319.7) |
| | | | | [symbol] and RELA, | |
| | | | | Highly expressed in T | |
| | | | | and B-cells | |
| | | | | SETDB2-PHF11 | DESCRIPTION: RecName: Full = PHD finger |
| | | | | naturally occurring | protein 11; AltName: Full = BRCA1 C-terminus- |
| | | | | readthrough | associated protein; AltName: Full = Renal |
| | | | | transcript | carcinoma antigen NY-REN-34; |
| | | | | [symbol] histone | FUNCTION: Positive regulator of Th1-type |
| | | | | methyltransferases | cytokine gene expression. |
| | | | | | SUBUNIT: Interacts with BRCA1 and RELA. |
| | ln 13/19 | | | | SETDB2-PHF11, SET2 = Tmethyl-CpG-binding |
| | | | | | domain (MBD) and a SET domain and function |
| | | | | | as histone methyltransferases. This protein is |
| | | | | | recruited to heterochromatin and plays a role |
| | | | | | in the regulation of chromosome segregation. |
| | | | | | This region is commonly deleted in chronic |
| | | | | | lymphocytic leukemia. Naturally-occuring |
| | | | | | readthrough transcription occurs from this |
| | | | | | gene to the downstream PHF11 (PHD finger |
| | | | | | protein 11) gene. Alternative splicing results |
| | | | | | in multiple transcript variants. [provided by |
| | | | | | RefSeq, Mar 2016] |
| | | | | | RefSeq: NR 135324.1 Status: Validated |
| | | | | | Description: SETDB2-PHF11 readthrough, |
| | | | | | transcript variant 2; This gene represents |
| | | | | | naturally-occurring readthrough transcription |
| | | | | | between the upstream SETDB2 (SET domain |
| | | | | | bifurcated 2) gene to the downstream PHF11 |
| | | | | | (PHD finger protein 11) gene. Readthrough |
| | | | | | transcripts may encode fusion proteins with |
| | | | | | similarity to proteins encoded by each of the |
| | | | | | individual genes or may encode candidates |
| | | | | | for nonsense-mediated decay |
| 3 | E | 5′Ferret/ | — | Interacts with GLUL = | PALMD (ENST00000263174.8) |
| | | 3′Arm | | biosynthesis of | |
| | | | | several amino acids, | |
| | | | | pyrimidines, and | |
| | | | | purine | |
| | | | | GLUL related | DESCRIPTION: RecName: Full = Palmdelphin; |
| | | | | pathways are TNFR1 | AltName: Full = Paralemnin-like protein; |
| | | | | Pathway | |
| | | | | Ubiquitous, | SUBUNIT: Interacts with GLUL (By similarity). |
| | | | | abundant in cardiac | |
| | | | | and skeletal muscle. | |
| | | | | | TISSUE SPECIFICITY: Ubiquitous. Most |
| | | | | | abundant in cardiac and skeletal muscle. |
| | | | | | GLUL (Glutamate-Ammonia Ligase) = catalyzes |
| | | | | | the production of glutamine and |
| | | | | | 4-aminobutanoate (gamma-aminobutyric acid, |
| | | | | | GABA) (Glutamine is an abundant amino acid, |
| | | | | | and is important to the biosynthesis of |
| | | | | | several amino acids, pyrimidines, and purine), |
| | | | | | Among GLUL related pathways are TNFR1 |
| | | | | | Pathway and Astrocytic Glutamate-Glutamine |
| | | | | | Uptake And Metabolism. Gene Ontology (GO) |
| | | | | | annotations related to this gene include |
| | | | | | identical protein binding and manganese ion |
| | | | | | binding. |
| 4 | ln 1/10 | Br Bat | +/−HEPG2 | may function in | FAM20A (ENST00000592554.1) |
| | sense | | | hematopoiesis. | |
| | ln 1/2 as | | | golgi associated | Description: Homo sapiens FAM20A, golgi |
| | | | | secretory pathway | associated secretory pathway pseudokinase |
| | | | | pseudokinase | (FAM20A), transcript variant 1, mRNA. (from |
| | | | | | RefSeq NM_017565) |
| | | | | | RefSeq Summary (NM_017565): This locus |
| | | | | | encodes a protein that is likely secreted and |
| | | | | | may function in hematopoiesis. |
| | | | | | DESCRIPTION: RecName: Full = Protein |
| | | | | | FAM20A; Flags: Precursor; |
| 5 | ln 2/13 | Manetee | ++HUV | bind to lipids such as | FNBP1L (ENST00000260506.12) |
| | | | EC, | phosphatidylinositol | |
| | | | HEPG2, | 4,5-bisphosphate | |
| | | | hESC, | (PIP2) | |
| | | | HSMM | promote membrane | DESCRIPTION: RecName: Full = Formin-binding |
| | | | | invagination and the | protein 1-like; AltName: Full = Transducer of |
| | | | | formation of tubules | Cdc42-dependent actin assembly protein 1; |
| | | | | | Short = Toca-1; |
| | | | | | FUNCTION: Required to coordinate |
| | | | | | membrane tubulation with reorganization of |
| | | | | | the actin cytoskeleton during endocytosis. |
| | | | | | May bind to lipids such as |
| | | | | | phosphatidylinositol 4,5-bisphosphate and |
| | | | | | phosphatidylserine and promote membrane |
| | | | | | invagination and the formation of tubules. |
| | | | | | Also promotes CDC42-induced actin |
| | | | | | polymerization by activating the WASL/ |
| | | | | | N-WASP-WASPIP/WIP complex, the |
| | | | | | predominant form of WASL/N-WASP in cells. |
| | | | | | Actin polymerization may promote the fission |
| | | | | | of membrane tubules to form endocytic |
| | | | | | vesicles. Essential for autophagy of |
| | | | | | intracellular bacterial pathogens. |
| 6 | ln 5/19 | Br Bat | — | endoproteolytic | PCSK5 (ENST00000545128.5) |
| | | | | processing for | |
| | | | | several integrin | |
| | | | | alpha subunits | |
| | | | | Expressed in | DESCRIPTION: RecName: Full = Proprotein |
| | | | | T-lymphocytes. | convertase subtilisin/kexin type 5; EC = 3.4.21.-; |
| | | | | | AltName: Full = Proprotein convertase 5; |
| | | | | | Short = PC5; AltName: Full = Proprotein |
| | | | | | convertase 6; Short = PC6; Short = hPC6; |
| | | | | | AltName: Full = Subtilisin/kexin-like protease |
| | | | | | PC5; Flags: Precursor; |
| | | | | | FUNCTION: Likely to represent a widespread |
| | | | | | endoprotease activity within the constitutive |
| | | | | | and regulated secretory pathway. Capable of |
| | | | | | cleavage at the RX(K/R)R consensus motif. |
| | | | | | Plays an essential role in pregnancy |
| | | | | | establishment by proteolytic activation of a |
| | | | | | number of important factors such as BMP2, |
| | | | | | CALD1 and alpha-integrins. |
| | | | | | TISSUE SPECIFICITY: Expressed in |
| | | | | | T-lymphocytes. |
| 7 | ln 5/8 | ard | ++++all | Essential component | SNAP23 (ENST00000249647.7) |
| | isoform | | | of high affinity | |
| | | | | receptor for the | |
| | | | | general membrane | |
| | | | | fusion machinery | |
| | 3′UTR | | | Cell membrane; | DESCRIPTION: RecName: Full = Synaptosomal- |
| | | | | Lipid-anchor. Cell | associated protein 23; Short = SNAP-23; |
| | | | | junction, synapse, | AltName: Full = Vesicle-membrane fusion |
| | | | | synaptosome | protein SNAP-23; |
| | | | | | FUNCTION: Essential component of the high |
| | | | | | affinity receptor for the general membrane |
| | | | | | fusion machinery and an important regulator |
| | | | | | of transport vesicle docking and fusion. |
| | | | | | SUBUNIT: Binds simultaneously to SNAPIN |
| | | | | | and SYN4. Found in a complex with VAMP8 |
| | | | | | and STX4 in pancreas. Interacts with STX1A |
| | | | | | and STX12 (By similarity). Binds tightly to |
| | | | | | multiple syntaxins and |
| | | | | | synaptobrevins/VAMPs. Found in a complex |
| | | | | | with VAMP8 and STX1A. |
| | | | | | TISSUE SPECIFICITY: Ubiquitous. Highest |
| | | | | | levels where found in placenta. |
| 8 | ln 1/31 | Guinea pig | ++GM78 | PI3K, classll with | PIK3C2A (RefSeq: NM_001321378.1) |
| | | | | calcium-dependent | |
| | | | | phospholipid binding | |
| | | | | motifs | |
| | | | | The PI3-kinase | Description: phosphatidylinositol-4-phosphate |
| | | | | activity of this | 3-kinase catalytic subunit type 2 |
| | | | | protein is not | alpha, transcript variant 2 |
| | | | | sensitive to | |
| | | | | nanomolar levels of | |
| | | | | the inhibitor | |
| | | | | wortmanin. | |
| | | | | activated by insulin | class II PI3-kinases. C2 domains act as |
| | | | | and may be involved | calcium-dependent phospholipid binding |
| | | | | in integrin-dependent | motifs that mediate translocation of proteins |
| | | | | signaling. | to membranes, and may also mediate |
| | | | | [provided by RefSeq, | protein-protein interactions. The PI3-kinase |
| | | | | July 2008]. | activity of this protein is not sensitive to |
| | | | | | nanomolar levels of the inhibitor wortmanin. |
| | | | | | This protein was shown to be able to be |
| | | | | | activated by insulin and may be involved in |
| | | | | | integrin-dependent signaling. [provided by |
| | | | | | RefSeq, July 2008]. |
| 9 | ln 3/17 | Chincilla | +/−all | Non-neuronal | MAP4 (ENST00000395734.7) |
| | | | | microtubule- | |
| | | | | associated protein. | |
| | | | | Promotes | |
| | | | | microtubule | |
| | | | | assembly | |
| | | | | | DESCRIPTION: RecName: Full = Microtubule- |
| | | | | | associated protein 4; Short = MAP-4; |
| | | | | | FUNCTION: Non-neuronal microtubule- |
| | | | | | associated protein. Promotes microtubule |
| | | | | | assembly. |
| | | | | | SUBUNIT: Interacts with SEPT2; this |
| | | | | | interaction impedes tubulin-binding. |
| 10 | ln 3/5 | arm | +++all | methylation of | DPY30 (ENST00000342166.9) |
| | | | | histone H3 at ‘Lys-4’, | |
| | | | | particularly | |
| | | | | trimethylation) = | |
| | | | | transcriptional | |
| | | | | activation | |
| | | | | subunit of the | DESCRIPTION: RecName: Full = Protein dpy-30 |
| | | | | [symbol] family of | homolog; AltName: Full = Dpy-30-like protein; |
| | | | | H3K4 | Short = Dpy-30L; |
| | | | | methyltransferases | |
| | | | | controls cell cycle | FUNCTION: As part of the MLL1/MLL |
| | | | | regulators, | complex, involved in the methylation of |
| | | | | proliferation and | histone H3 at ‘Lys-4’, particularly |
| | | | | differentiation of | trimethylation. Histone H3 ‘Lys-4’ methylation |
| | | | | hematopoietic | represents a specific tag for epigenetic |
| | | | | progenitor cells | transcriptional activation. May play some role |
| | | | | | in histone H3 acetylation. In a |
| | | | | | teratocarcinoma cell, plays a crucial role in |
| | | | | | retinoic acid-induced differentiation along the |
| | | | | | neural lineage, regulating gene induction and |
| | | | | | H3 ‘Lys-4’ methylation at key developmental |
| | | | | | loci. May also play an indirect or direct role in |
| | | | | | endosomal transport. |
| | | | | May play some role | SUBUNIT: Homodimer. Core component of |
| | | | | in histone H3 | several methyltransferase- containing |
| | | | | acetylation (Met and | complexes including MLL1/MLL, MLL2/3 (also |
| | | | | Ac?) | named ASCOM complex) and MLL4/WBP7. |
| | | | | | MEN1, HCFC1, HCFC2, NCOA6, KDM6A, |
| | | | | | PAXIP1/PTIP, PAGR1 and alpha- and beta- |
| | | | | | tubulin (By similarity). Interacts with ASH2L; |
| | | | | | the interaction is direct. Interacts with |
| | | | | | ARFGEF1. Component of the SET1 complex, at |
| | | | | | least composed of the catalytic subunit |
| | | | | | (SETD1A or SETD1B), WDR5, |
| | | | | crucial to retinoic | INTERACTION: Self; NbExp = 3; IntAct = |
| | | | | acid-induced | EBI-744973, EBI-744973; Q9UBL3:ASH2L; |
| | | | | differentiation along | NbExp = 5; IntAct = EBI-744973, EBI-540797; |
| | | | | the neural lineage | |

It will be understood that open-source algorithms such as BLAT2013 or BLASTn may be sometimes changed without notification. Therefore, the alignment rankings reported herein may differ between algorithms and may change over time; however, the overall pathway defined by genes aligned by the method disclosed herein remains the same.

The percent identity rankings differed between algorithms; however, it did not matter which algorithmic ranking system was used, human BLAT and BLASTn alignments ultimately converged on the same pathway.

The highest identity alignments (as defined above) were evaluated for genomic position and, if within the regulatory regions of a known gene, their function identified using Weismann Institute of Science database (“GeneCards.org”).

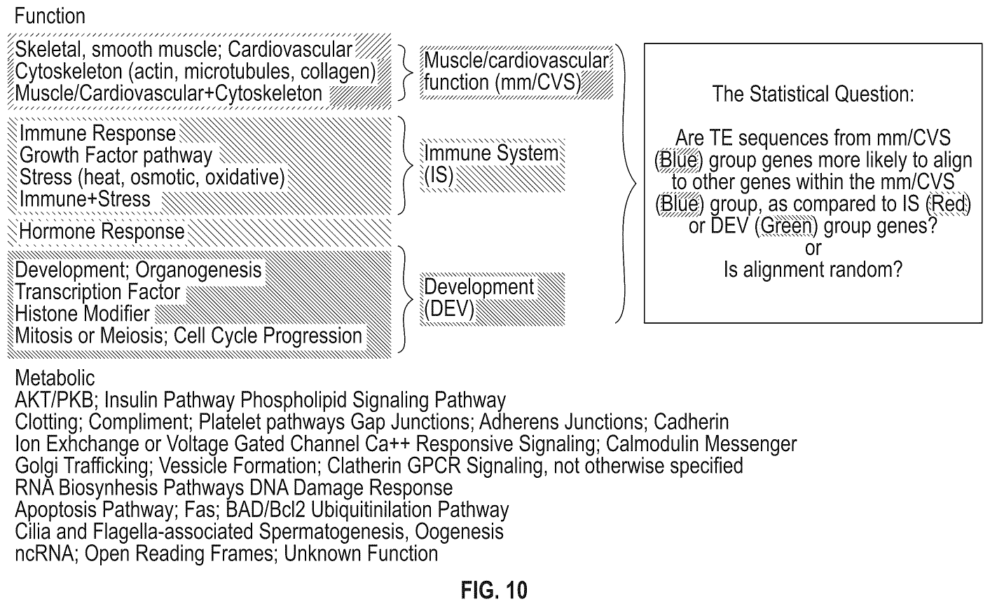

If alignments are within the regulatory regions of a coding or noncoding gene, the full function of that gene is tabulated, using a detailed gene database (e.g., GeneCard.com, Weismann Institute), to the extent that it is known. Functional Categories used herein are presented in FIGS. 8, 10 and Table 3.

The process is then repeated with TEr sequences found in cis.

To further expand the network, the Method can be repeated with TEr sequences of the functionally-grouped aligned genes thus creating a “neural-type” network (FIG. 4).

### Example 3: Bioinformatics Study

Genomic alignments were tested among computer-generated random sequences (N=50, 20nt each; generated using the sample function in the R language (R-project.org R-project.org). There were no alignments among them.

TEr selected randomly were then tested for genomic alignments (N=25; blinded selection) aligned with high-identity (top 10 BLAT₂₀₁₃ alignments) as per the Method. Not all random TEr (N=25) aligned 10 times within the genome, leading to 240 total genomic alignments (Table 3). Interestingly, random TEr tended to align within gene regulatory regions, consistent with previous observations that TEr positions are not randomly distributed.

TABLE 3
List of Functional categories and the Rates at Which Random TEr
Align to Genes Within Them

| | | | |
|---|---|---|---|
| RANDOM TEr | | 25 | |
| Total alignments | | 240 | |
| Intergenic (IG) | | 33 | 13.8% |
| alignments | | | |
| Gene alignments | | 207 | 86.3% |
| | lncRNA | 37 | 17.9% |
| | Unknown coding genes | 30 | 14.5% |
| | Known coding genes | 140 | 67.6% |
| Function of known | | | |
| coding genes | | | |
| | Growth Factor | 19 | 13.6% |
| | Transcription | 18 | 12.9% |
| | Metabolic | 17 | 12.1% |
| | Immune response | 12 | 8.6% |
| | Cell motility, cytoskeletal | 11 | 7.9% |
| | Mitosis | 9 | 6.4% |
| | Vesicle movement | 9 | 6.4% |
| | RNA biosynth, processing | 6 | 4.3% |
| | Neural specific | 6 | 4.3% |
| | mm/CVS, Angiogenesis | 5 | 3.6% |
| | Ubiquitin | 4 | 2.9% |
| | Sperm/Ovary specific | 4 | 2.9% |
| | Cell adhesion/gap junctions | 3 | 2.1% |
| | Stress Response | 3 | 2.1% |
| | Hormonal | 3 | 2.1% |
| | Voltage channels | 3 | 2.1% |
| | Developmental | 3 | 2.1% |
| | Coagulation | 2 | 1.4% |
| | Phospholipid Signaling | 1 | 0.7% |
| | Apoptosis | 1 | 0.7% |
| | Insulin, Alt, glucose | 1 | 0.7% |

A bioinformatics study was performed testing the hypothesis that TEs disperse high identity variant sequence to functionally grouped genes. The fraction of Index TEr alignments to genes of a specific function were compared between three biologic groups: Muscle/Cardiovascular system (mm/CVS), Developmental system (DEV) and Immune system (IS) (Table 4).

For each biologic system, 4 key genes (Index genes) were chosen to represent that system, and for each Index gene, 7 TEr chosen (Table 4).

TABLE 4
Summary of Bioinformatics study design

| | | #TE | Max BLAT | Max BLAT |
|---|---|---|---|---|
| System of | | per | alignments | alignments |
| Interest | Key Genes | gene | per TE | per system |
| Immune system | 1. GR | 7 | 10 | |
| | 2. CRHR2 | 7 | 10 | |
| | 3. NFκB | 7 | 10 | |
| | 4. TLR3 | 7 | 10 | 280 |
| Muscle/ | 1. MyoD | 7 | 10 | |
| Cardiovascular | 2. TPM1 | 7 | 10 | |
| | 3. CALDS | 7 | 10 | |
| | 4. CKM | 7 | 10 | 280 |
| Developmental | 1. Promoter region #1 | 7 | 10 | |
| | 2. Enhancer region #2 | 7 | 10 | |
| | 3. Enhancer region #3 | 7 | 10 | |
| | 4. Enhancer region #4 | 7 | 10 | 208 |

```
TEs (N = 7/gene)
4 key genes/system
3 biologic systems
  Muscle/Cardiovascular (mm/CVS)
  Immune System (IS)
  Development (DEV)
```

The summary of the statistical analysis is presented in FIG. 10. The fraction of index TEs positive for each function was compared between the three biologic groups with both parametric (t test with pooled variance) and nonparametric (Kruskal-Wallis) tests (Table 5). The match of the index TEr with itself was not included in calculations. P values are reported without correction for multiple comparisons.

TABLE 5
Results of Bioinformatics Study.

| | IS vs | | mm/CVS | | IS | |
|---|---|---|---|---|---|---|
| | mm/CVS | | vs DEV | | vs DEV | |
| | KW. | t. | KW. | t. | KW. | t. |
| FUNCTION | test | test | test | test | test | test |
| Skeletal, smooth muscle; Cardiovascular | 0.002 | 0.008 | 0.001 | 0.008 | 0.69 | 0.96 |
| Cytoskeleton (actin, microtubules, collagen) | 0.09 | 0.14 | 0.01 | 0.04 | 0.45 | 0.52 |
| Muscle/Cardiovascular + Cytoskeleton | 0.0002 | 0.002 | 0.00083 | 0.0 [symbol] 1 | 0.53 | 0.74 |
| Immune Response | 0.10 | 0.07 | 0.28 | 0.12 | 0.69 | 0.89 |
| Growth Factor pathway | 0.15 | 0.11 | 0.003 | 0.02 | 0.08 | 0.08 |
| Stress (heat, [symbol] , oxidative) | 0.97 | 0.70 | 0.54 | 0.54 | 0.52 | 0.40 |
| Immune + Stress | 0.08 | 0.11 | 0.22 | 0.19 | 0.63 | 0.88 |
| [symbol] ; Organogenesis [symbol] | 0.28 | 0.28 | 0.0 [symbol] | 0.02 | 0.003 | 0.007 |
| Transcription Factor | 0.34 | 0.39 | 0.95 | 0.96 | 0.31 | 0.3 [symbol] |
| [symbol] | 0.08 | 0.08 | 0. [symbol] | 0. [symbol] 4 | 0.73 | 0.73 |
| Mitosis [symbol] ; Cell Cycle Progression | 0.12 | 0.12 | 0.72 | 0.58 | 0.23 | 0.38 |
| Metabolic | 0.35 | 0.20 | 0.88 | 0.48 | 0.49 | 0.60 |
| AKT/PKB [symbol] Insulin Pathway | 1.00 | 1.00 | 0.56 | 0.52 | 0.56 | 0.52 |
| Phospholipid Signaling Pathway | 0.32 | 0.32 | 0.16 | 0.16 | 0.58 | 0.58 |
| Clotting; Compliment; Platelet pathways | 0.15 | 0.16 | all values 0 | NA | 0.15 | 0.15 |
| Gap Junctions; Adherens Junctions; Cadherin | 0.17 | 0.22 | 0.28 | 0.31 | 0.74 | 0.81 |
| Ion Exchange or Voltage Gated Channel | 0.33 | 0.79 | 0.96 | 0.52 | 0.2 [symbol] | 0.24 |
| Ca++ Responsive Signalling; C [symbol] Messenger | 0.30 | 0.31 | 0.07 | 0.07 | 0.31 | 0.31 |
| Golgi Traff [symbol] ing; Vessicle Formation; C [symbol] | 0.72 | 0.72 | 0.66 | 0.33 | 0.44 | 0.24 |
| GPCR Signaling, not otherwise specified | 1.00 | 1.00 | 0.15 | 0.15 | 0.15 | 0.15 |
| RNA Biosynthesis Pathways | 1.00 | 1.00 | 0.30 | 0.16 | 0.30 | 0.16 |
| DNA Damage Response | 0.30 | 0.28 | 0.59 | 0.55 | 0.5 [symbol] | 0.5 [symbol] |
| Apoptosis Pathway; Fas; B [symbol] | 0.15 | 0.16 | 0.65 | 0.49 | 0.08 | 0.10 |
| [symbol] Pathway | 0.32 | 0.32 | 0.32 | 0.33 | 0.08 | 0.08 |
| [symbol] and [symbol] -associated | 0.15 | 0.16 | all values 0 | NA | 0.15 | 0.15 |
| [symbol] genesis, [symbol] genesis | 0.69 | 0.69 | 0.96 | 0.96 | 0.65 | 0.66 |
| [symbol] RNA; Open Reading Frames; Unknown Function | 0. [symbol] 6 | 0. [symbol] 4 | 0.31 | 0.26 | 0.41 | 0.27 |

TEr of MyoD have a higher likelihood of aligning TE of genes of mm/CVS pathways
TEr of HOXA have a higher likelihood of aligning TE of genes of DEV pathways
TEr of hormone receptors have a higher likelihood of aligning TE of genes of hormonal pathways [symbol]
indicates data missing or illegible when filed

The trial was terminated at 4 Index genes/system and 7 Index TEr/gene (280 TEr maximal alignments per biologic system) when strong statistical significance became apparent (Table 5).

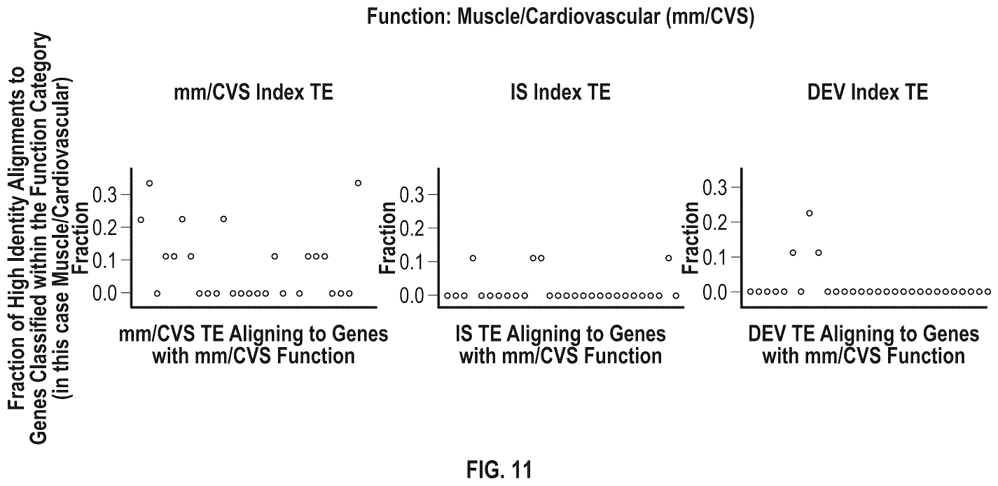

Unexpectedly, Index genes representing each biologic system had a high likelihood of sharing high-identity TEr (within the top ten BLAT₂₀₁₃ alignments) (Table 5). For example, contrary to expectation, TEr sequences from regulatory DNA of genes key to the Muscle/Cardiovascular (mm/CVS) and Developmental (DEV) biological pathways were significantly more likely to align with high-identity to genes participating in the same pathway as compared to the genes aligned by those of a different biologic pathway (FIG. 11, Table 5 second row). The choice of Immune System (IS) key genes included two hormone receptors activated by inflammation and stress (Glucocorticoid receptor and CRH Receptor 2) and the likelihood of the IS group of Index TEr aligning to genes participating in hormonal pathways was significantly higher than those of mm/CVS index TEr (P<0.04) or DEV index TEr (P<0.004). Other results unlikely to be random included examples of single genes targeted multiple times by Index TEr from a gene in the same biologic pathway and single Index TEr that aligned with high identity to multiple functionally-linked genes (described in detail in Examples below).

Index TEr of all three functional groups matched in similar fractions to all other functional categories (Table 5, row 11 onwards), including Immune function genes. The background rate of alignment of random TEr to Immune genes was high (8.6%; Table 3) as compared to the rate at which they aligned to mm/CVS or DEV genes (3.6% and 2.1% respectively).

Shared high-identity sequences ranged in length from 20 bp to hundreds of base pairs. They did not necessarily include transcription-factor binding sites and were often transcribed in cell-type specific patterns into RNA fragments unrelated to transposition. They were not classified as “miRNA”, “tRNA”, eRNA or “piRNA”. Alignments were not pericentromeric and rarely in 3′UTR of coding-genes. All TE families and subtypes were represented in percentages consistent with their reported frequency in the human genome.

In summary, key muscle/cardiovascular system genes were found to have a higher likelihood of aligning to Ter of other muscle genes. Key developmental genes were found to have a higher likelihood of aligning to Ter of other developmental genes. TEr of immune system genes were found to align equally between groups. Baseline rate of IS alignment using random TEr is high.

### Examples 4: TEr Alignments of Hub Genes

TEr alignments of pathway hub genes within different biologic systems were studied in greater detail with the in silico method (Table 6).

TABLE 6
Additional examples of hub genes tested for network
discovery using in silico method

| Ex: | Hub genes | Pathway |
|---|---|---|
| 3 | NFkB1 and its cis | 1. Phospholipid signaling- |
| | lncRNA_{LOC105377621/RP11-499E18.1} | mediated cell activation |
| | | 2. Epithelial to mesenchymal |
| | | transition (EMT) |
| | | In vitro data is presented |
| | | about the participation of |
| | | lncRNA_{LOC105377621/RP11-499E18.1} |
| | | TEr in EMT |
| 4 | MyoD1 and its cis | Myogenesis |
| | lncRNA_{AC124301.1} | |
| 5 | SRA1 lncRNA | Parkinson's disease |
| 6 | NFkB1 promoter NPtx and | RNA-binding proteins |
| | promoter ncRNA | required for RNA |
| | AF213884.2 | transcription, formation |
| | | and packaging |
| 7 | FAK/PTK2, b-Catenin | Epithelial to mesenchymal |
| | and Wnt | transition |
| 8 | CRHR2 | Stress-related lipid metabolism |
| 9 | CD4 | T_{H} immune cell activation, |
| | | HIV binding |

### Example 5: Nuclear Factor-Kappa B Subunit 1 (NFkB1) TEr and Genes Coordinating Cell Activation and Tumorigenesis

NFkBT is a 105 kD protein which undergoes cotranslational processing to produce a 50 kD protein which is the DNA binding subunit of the NF-kappa-B (NFKB) protein complex. Its most common partner is subunit p65: RELA. NFkB links signal transduction events initiated at the cell membrane by a vast array of stimuli (cytokines, oxidant-free radicals, bacterial/viral products), translocating the signal to the nucleus where it directly binds to genes that coordinate inflammation, immunity, differentiation, cell growth, tumorigenesis and apoptosis.

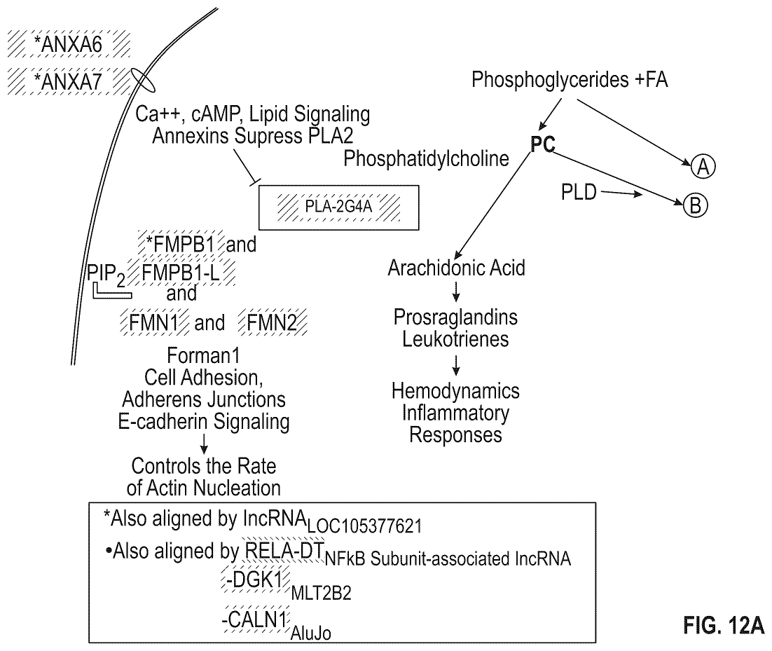

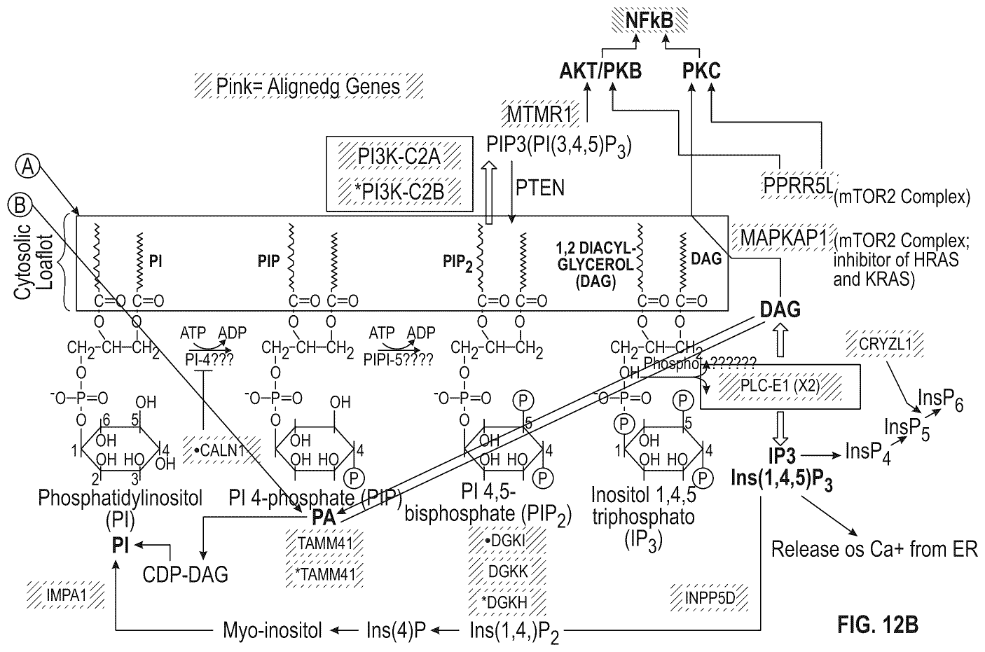

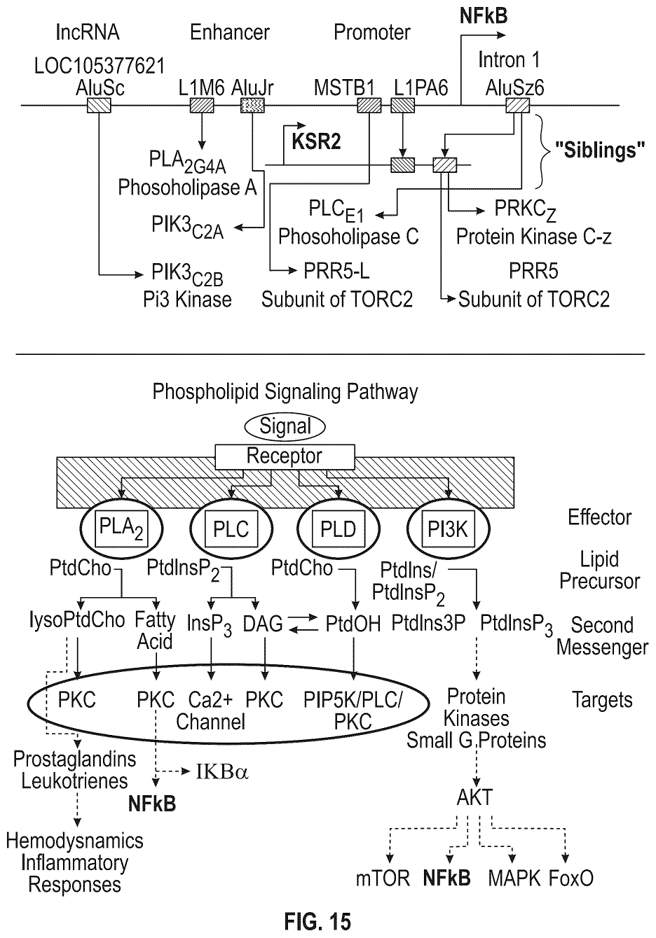

There was significant likelihood that TEr within NFkBT transcriptional regulatory regions share high-identity TEr with phospholipid signaling pathway-specific genes, an ancient pathway critical to the genes critical to the initiation of cell activation at the plasma membrane (FIGS. 12, 15, Table 7).

TABLE 7
Significant likelihood that the results are specific and non-random

| | | | |
|---|---|---|---|
| Index Gene | TEr | n/N | P value |

Likelihood that NFkB1 TEr align to Phospholipid
Signaling Pathway Genes

| | | | |
|---|---|---|---|
| NFkB | 41 | 17/367 | |
| Random TE | 25 | 1/240 | <0.003 |
| Hair genes Control | 28 | 2/270 | <0.004 |
| Housekeeping genes Control | 28 | 2/247 | <0.007 |

Likelihood that MyoD1 TEr align to
Muscle/Cardiovascular Pathway Genes

| | | | |
|---|---|---|---|
| MyoD1 | 46 | 48/446 | |
| Random TE | 25 | 5/240 | <0.00004 |
| Hair genes Control | 28 | 10/270 | <0.0008 |
| Housekeeping genes Control | 28 | 6/247 | <0.00009 |

n = # TEr alignments to specific pathway genes
N = Total TEr with high identity alignments
Abbreviations: NFkB1: Nuclear Factor Kappa B Subunit 1; a transcription factor that is the endpoint of a series of signal transduction events that are initiated by stimuli related to embryogenesis, oncogenesis, cell activation, inflammation, and cell growth. MyoD1: Myogenic Differentiation 1 promotes transcription of muscle-specific target genes and plays a role in muscle differentiation.

BLAT₂₀₁₃ analysis of promoter, promoter-proximal intron 1 and highly conserved enhancer TEr sequences of NFkB1 (N=41, Total alignments=367) revealed a significantly larger fraction of TEr sequences aligned with high-identity to genes of the Phospholipid-mediated signaling cascade (N=17) than did random TEr (P<0.003), Hair gene-specific TEr (P<0.004) or TEr of Housekeeping genes (P<0.007) (Table 7). This is in contrast to TEr of the key gene of muscle development MyoD1, with aligned with high likelihood to genes of the muscle/cardiovascular system.

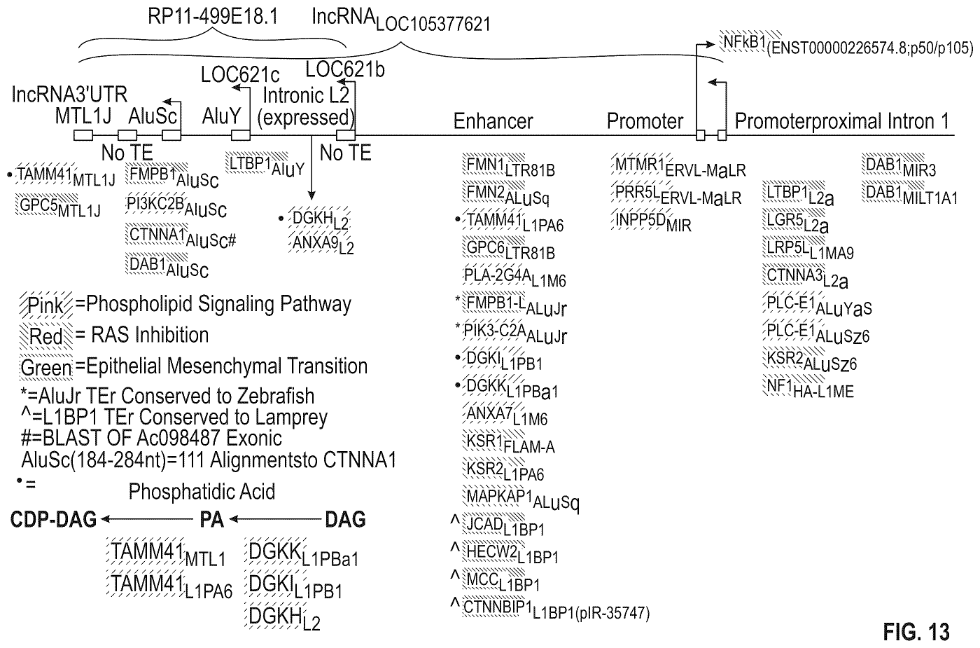

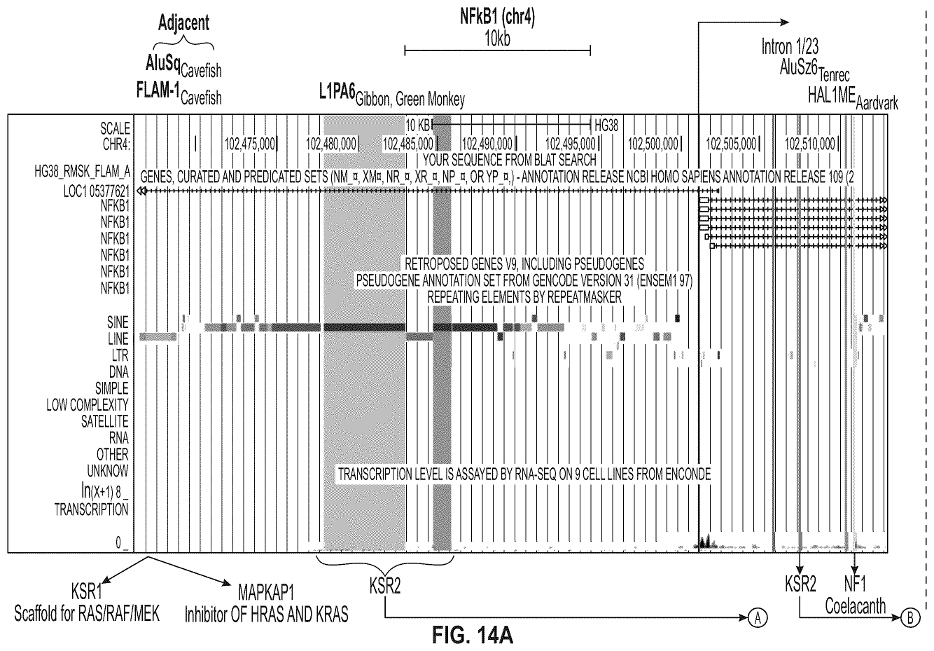

The ancient Phospholipid Signaling Pathway is initiated by inflammatory and proliferative signals that activate cell membrane phospholipids, triggering immediate intracellular release of Ca²⁺ and the phosphorylation of effector proteins that activate NFkB1, (FIG. 12; outlined in FIG. 15). Multiple genes encoding isoforms of key proteins critical to the initiation of phospholipid signaling were aligned by NFkB TEr including PI3-Kinase (PI3K-C2A), Phospholipase A (PLA2G4A) and Phospholipase C (PLC-E1) (FIG. 12). TEr with high identity to genes of this pathway were present throughout KFkB1 transcriptional regulatory regions including its upstream lncRNA_{LOC105377621/RP11-499E18.1} (FIG. 13). Astonishingly, PLC-E1 was aligned by two different Alu Repeats in the promoter-proximal region of NFkB1 intron 1: AluYa5 and AluSz6 _{chr4:102507477-102507601} (which also aligned KSR2, see below). Index TEr aligned to three genes encoding enzyme isoforms responsible for Phosphatidic Acid (PA) metabolism to DAG (Diacylglycerol Kinase Iota, Kappa and Eta; DGKI, DGKK and DGKH; and aligned another gene of this same pathway twice: TAMM41 (Mitochondrial Translocator Assembly and Maintenance Homolog; catalyzes the reaction of PA to CDP-diacylglycerol (CDP-DAG) (FIG. 13). Interestingly, RELA/p65 (most common NFkB1/p50 subunit within the NFkB complex) contained a promoter TEr that also aligned to the DGK1 gene.

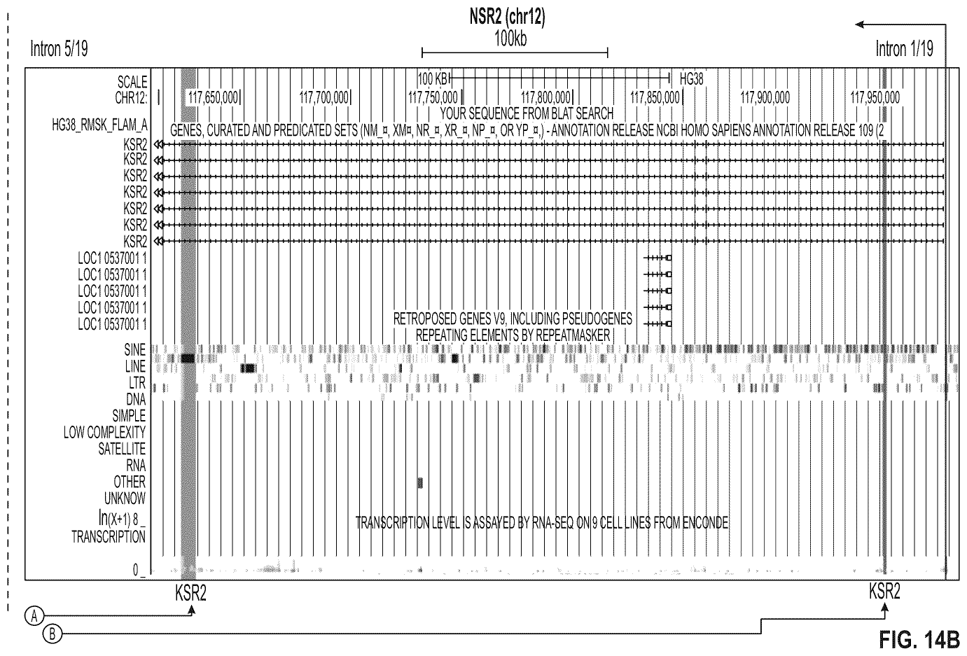

Other results unlikely to be random included five NFkB1 TEr sequences that align with high identity to four genes encoding key inhibitors of the Ras signal transduction pathway (critical molecular switch that turns on various target proteins necessary for cellular proliferation) (FIG. 13, 14). KSR2 (Kinase Suppressor of Ras 2) is aligned twice (FIG. 14). Interestingly, the “sibling” TEr within KSR2 further aligned to genes critical to the phospholipid signaling pathway (FIG. 15). The family of Ras proteins play a pivotal role in the regulation of cell proliferation and their activation is critical to downstream NFkB1-mediated pathway outcome and to cell oncogenic potential. Intron 1 TEr also aligned Neurofibromin 1 (NF1; negative regulator of the Ras signal transduction pathway) and both an enhancer and intron 1 TEr aligned KSR2 (FIG. 13). Kinase Suppressor of Ras 1 (KSR1: a MEK/RAF/RAS scaffold) was aligned by a conserved enhancer NFkB1 TEr, as was MAPKAP1 (subunit of nutrient-insensitive mTOR2, inhibits HRAS and KRAS) which, astonishingly, was directly adjacent to the KSR1-aligning TEr. In total, five NFkB1 index TEr sequences aligned to four genes encoding RAS inhibitors.

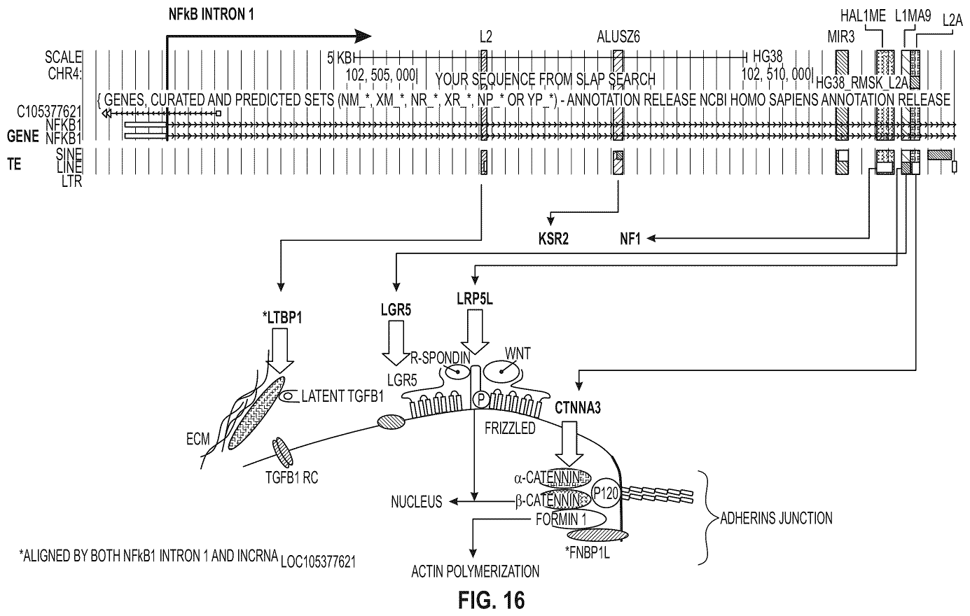

The first set of TEr following the NFkB1 5′UTR in intron 1 is especially interesting: not only do TEr aligning KSR2 and NF1 lie close together, this region contained several sequential TEr that aligned with high identity to genes critical to the initiation of EMT at the plasma membrane (FIG. 16). FIG. 16 also highlights the Adherens Junction, where genes essential to initiating and maintaining cell-cell contact are aligned by TEr of NFkBT, including both Formin 1 and 2 (FMN1, 2; essential for polymerization of linear actin cables; conserved to slime mold) as well as two of Formin's binding proteins (FNPB1 and FNPB1-L). Promoter-proximal intron 1 RNA sequences are transcribed soon after RNA polymerase II has begun mRNA elongation. While the 5′untranslated region (UTR; exon 1) forms secondary RNA structures required for mRNA capping and translation, the intronic region that follows is not known to participate in RNA-mediated signaling. Whether RNAs from these TEr sequences are physiologically active is may require additional investigation.

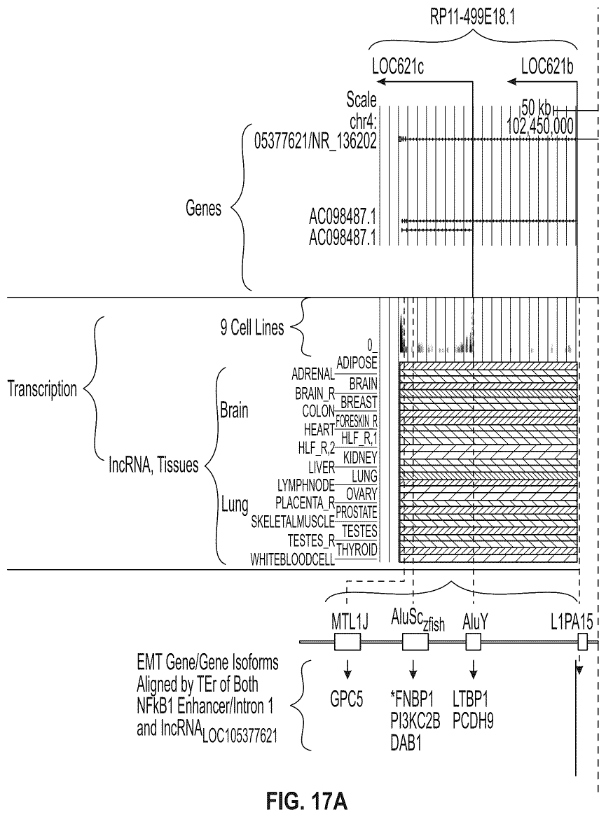

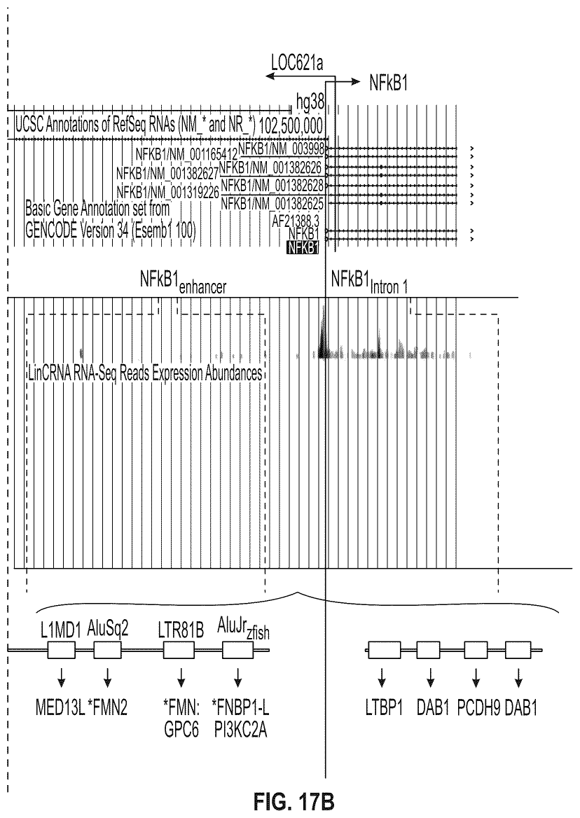

Importantly, there were several genes aligned by TEr of both NFkB1 enhancer/intron 1 TEr and lncRNA_{LOC105377621/RP11-499E18.1} TEr (FIG. 17; Table 8). For example, DAB1 (Disabled (Drosophila) Homolog 1) was aligned 3 times: twice by adjacent TEr of NFkB1 intron 1 and once by an exonic TEr of lncRNA_{LOC105377621/RP11-499E18.1} (FIG. 17; Table 8. DAB1 is activated upon the binding of Reelin, which is expressed most strongly in brain, blood and liver. It increases with liver damage, returning to normal following its repair, and it is elevated in aggressive pancreatic cancer.

TABLE 8
Exonic TEr of lncRNA_{LOC105377621/RP11-499E18.1} that aligned the same
genes as TEr from NFkB1 enhancer/intron 1

| | | |
|---|---|---|
| NFkB1 | | |
| Enh, intron 1 | lncRNA_{LOC105377621} | TEr-aligned Genes/Gene isoforms |

TEr alignments to same gene_{(TEr subscript to aligned gene)}

| | | |
|---|---|---|
| LTBP1 | LTBP1 | Latent-Transforming Growth Factor Beta-Binding Protein 1: controls TGF-beta activation |
| 12a to ln 3/33 | AluY to ln 5/33 | |
| DAB1 | DAB1 | Disabled (Drosophila) Homolog 1-Reelin Signal Transducer 1: |
| 1. MIR3 to Enh | AluSc, | Activated by the binding of Reelin (secreted by developing neurons, liver, pancreas) to |
| 2. MLT1A1 to | numerous | VLDLR and LRP8/APOER2; triggers activation of Src kinases, PI3K and Crk (cell |
| ln 2/14 | alignments | adhesion, spreading and migration). Loss of Reelin contributes to the ability of |
| | | pancreatic cancer cells to migrate and invade |
| PCDH9 | PCDH9 | Protocadherin-9: calcium-dependent cell-adhesion protein, involved in signaling at |
| L2a to ln 2/4 | AluY to ln 3/4 | neuronal synaptic junctions |
| MED13L | MED13L | Mediator complex subunit 13 like: transcriptional coactivator for most RNA polymerase |
| L1MD1 to | L1PA15 to Enh | II-transcribed genes (participates in enhancer clusters). This subunit may specifically |
| ln 2/30 | | regulate transcription of targets of the Wnt signaling pathway and SHH signaling pathway |

TEr alignments to Isoforms

| | | |
|---|---|---|
| FNBP1L | FNBP1 | Formin-binding protein 1 and FBP1-Like: binds PIP2 and Formin (aligned by two NFkB1 |
| (AluJr) | (AluSc) | enhancer TEr; conserved to slime mold, polymerization of linear actin cable in formation of |
| | | adherens junction, regulates the shape and position of the nucleus during cell migration) |
| PI3KCZA | PI3KC2B | Phosphatidylinositol-4-phosphate 3-kinase with C2 domain (Type II): key role in signaling |
| (AluJr) | (Alusc) | pathways involved in cell activation and proliferation, oncogenic transformation, cell |
| | | survival and cell migration |
| GPC6 | GPC5 | Glypican 5, 6: cell surface heparan sulfate proteoglycan coreceptors for growth factors. |
| (CTR81B) | (MLT1J) | Associated with Wnt signaling |

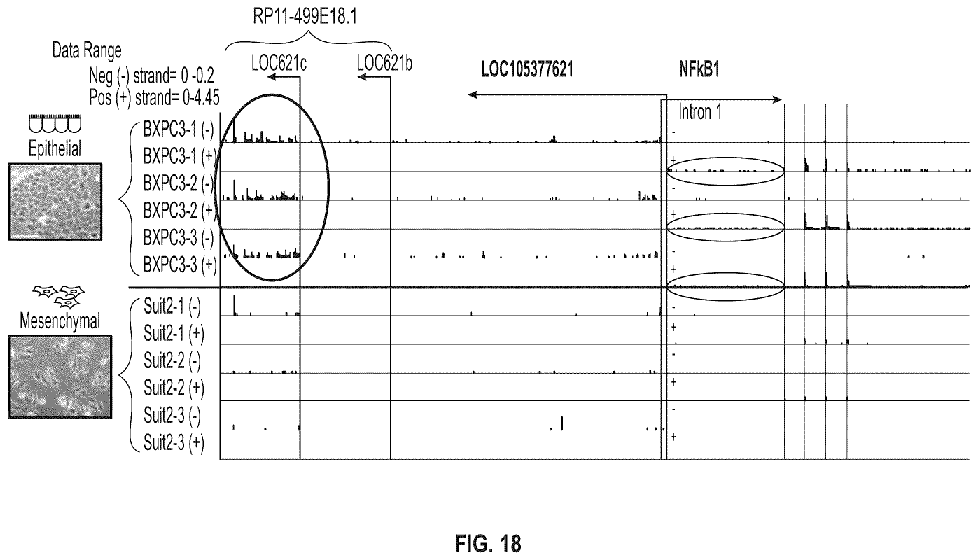

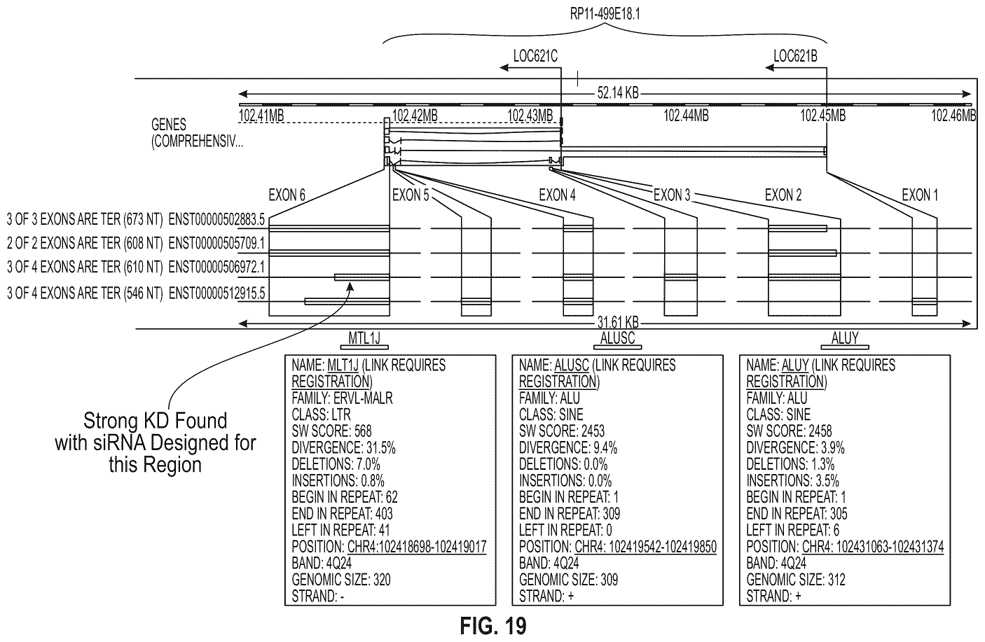

This convergence of TEr alignments to genes critical to the initiation of EMT led us to analyze the expression ofNFkB1 and lncRNA_{LOC105377621} isoforms (also termed RP11-499E18.1) in cancer cells. Using the public Gene Expression Omnibus high RNAseq profiling database, pancreatic adenocarcinoma cell lines were assayed for NFkB1 intron 1 and RP11-499E18.1 expression (GSE88759) (Barrett T, Wilhite S E, Ledoux P, Evangelista C, Kim I F, Tomashevsky M, et al. NCBI GEO: archive for functional genomics data sets-update. Nucleic Acids Research. 2012; 41(D1):D991-D5.) Both were expressed in a well differentiated (epithelial) pancreatic cancer cell line (BxPC3) and markedly decreased in a less differentiated (mesenchymal) cell line (S2-007/Suit2), suggesting their loss is associated with tumor progression (FIG. 18). In vitro analysis of RP11-499E18.1 was performed in PDA cell lines BxPC3, Suit2, Pancr1 and COLO357 (also associated with metastasis). RP11-499E18.1 is the UCSC term used for several isoforms, here distinguished as isoforms LOC621b and c; FIG. 19). Isoforms range in size from 608-673nt with LOC621c isoforms initiating with an AluY fragment and terminating in an MTL1J fragment. Depending on the isoform, 2 of 2, 3 of 3 or 3 of 4 exons consist of TEr sequences (FIG. 19). Genes to which these TEr sequences align within phospholipid signaling or EMT pathways are listed in FIG. 13.

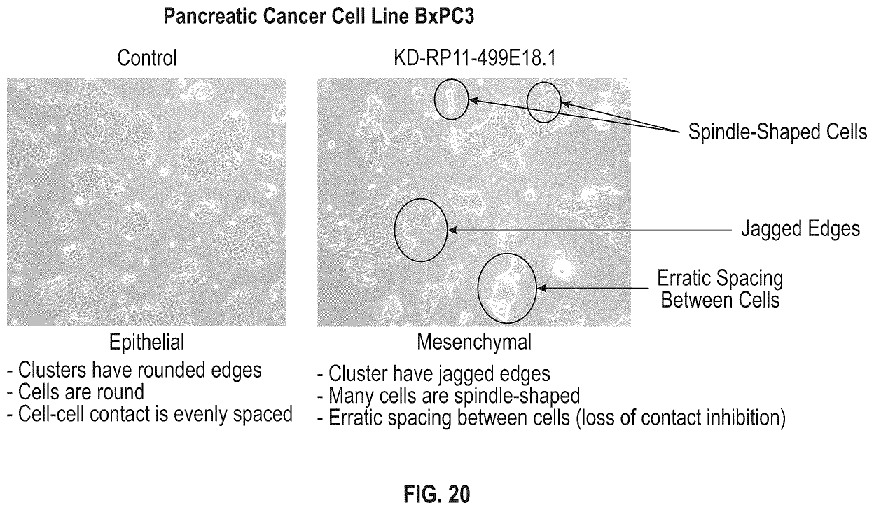

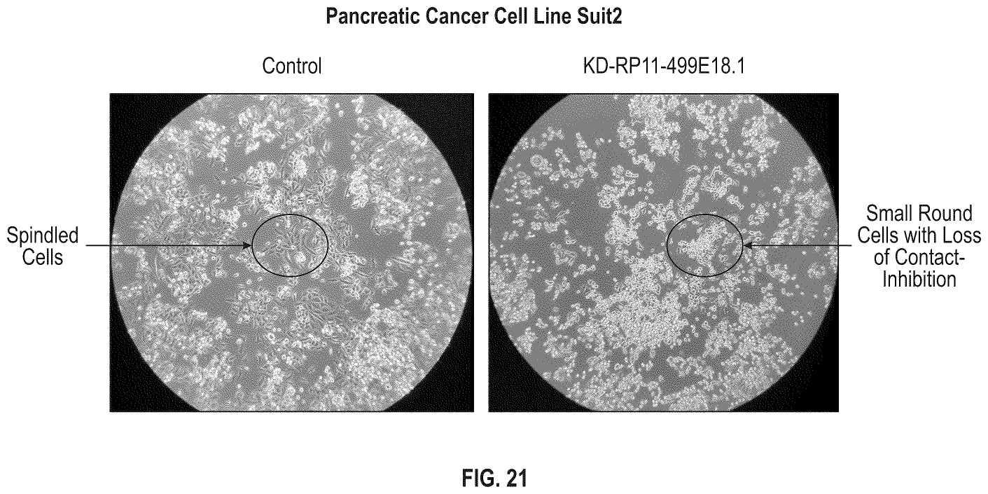

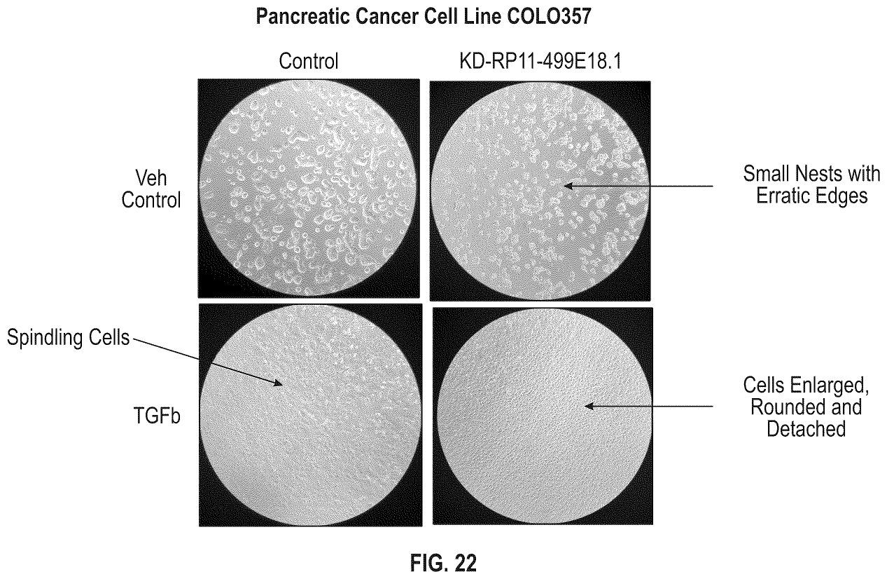

SiRNA sequence was designed to the 3′ MTL1J. Knock down (KD) of RP11-499E18.1 resulted in dramatic phenotypic changes in all PDA cell lines (FIGS. 20-22). Following KD, the well differentiated epithelioid cell line BxPC3-KD exhibited morphologic changes from epithelioid to mesenchymal, (FIG. 20) as did Pancr1-KD. In contrast the highly aggressive cell line Suit2-KD transitioned from a mix of poorly-differentiated and spindling cells into small round cells with no apparent contact-inhibition (FIG. 21). COLO357-KD transitioned from predominantly nested epithelioid cells into ragged clusters of small round cells (FIG. 22). PCR analysis of COLO357-KD cells revealed a marked decrease in markers of both mesenchymal (CDH2, VIM, SNAI) and epithelial (CDH1) differentiation (Table 9). TGFb stimulation of COLO357-KD cells resulted in round cell enlargement and marked loss of cell-to-cell contact inhibition. These TGFb stimulated COLO357-KD showed a strong increase in the mesenchymal-cell marker VIM, but the cells did not show and increase in SNAI1 or the typical spindle pattern of EMT (FIG. 22). Interestingly, in TGFb controls, RP11-499E18.1 levels doubled over baseline, suggesting its participation in TGFb-stimulated cell responses; however, in its absence, the EMT-associated mesenchymal phenotype appeared to further de-differentiate, possibly into cancer stem cells.

TABLE 9
Fold changes in RNA expression (as compared to control)
of EMT Markers in COLO357 cells following
RP11-499E18.1 knock down and TGFb stimulation.

| | | siRNA | | | |
|---|---|---|---|---|---|
| | | − | − | + | + |
| | | TGFb | | | |
| | | − | + | − | + |
| | RP11-499E18.1 | 1 | 2.2 | 0.2 | 0.3 |
| Epithelial | CDH1 | 1 | 1.4 | 0.5 | 0.3 |
| Mesenchymal | CDH2 | 1 | 5.2 | 0.4 | 1.6 |
| | VIM | 1 | 18.9 | 0.8 | 17.7 |
| | SNAI1 | 1 | 1.7 | 0.5 | 0.4 |
| | ZEB1 | 1 | 1.4 | 1.2 | 1.3 |

Green = increased, Red = decreased, Purple = decreased with ratio of CDH2:CDH1 consistent with EMT transition

The full identity of the small round cells seen in Suit2 and COLO357 following RP11-499E18.1 siRNA awaits RNAseq results (pending). However, the decrease of both epithelial and mesenchymal cell markers suggests a transition to—(or selection for—) a cancer stem-cell type. The potent de-differentiation effects seen with the loss of this single small lncRNA, which consists predominantly of TEr that align genes of EMT, suggest that RP11-499E18.1 is behaving like a molecule required for maintenance of cell differentiation; in its absence, well differentiated epithelioid tumors transition into mesenchymal and poorly differentiated tumors completely de-differentiate. Results of RP11-499E18.1 overexpression experiments are pending.

Our findings in pancreatic adenocarcinoma cell lines differed somewhat from those of Yang et al, who report that RP11-499E18.1 expression is decreased in ovarian cancer tissue associated with rapid progression. (Yang J, Peng S, Zhang K. LncRNA RP11-499E18.1 Inhibits Proliferation, Migration, and Epithelial-Mesenchymal Transition Process of Ovarian Cancer Cells by Dissociating PAK2-SOX2 Interaction. Front Cell Dev Biol. 2021; 9:697831.) RP11-499E18.1 knock down in OC cells increased cell proliferation, migration, colony formation, and EMT transformation, and RP11-499E18.1 overexpression reversed these effects. (Yang J, Peng S, Zhang K. LncRNA RP11-499E18.1 Inhibits Proliferation, Migration, and Epithelial-Mesenchymal Transition Process of Ovarian Cancer Cells by Dissociating PAK2-SOX2 Interaction. Front Cell Dev Biol. 2021; 9:697831.) These authors do not note the dramatic change in cell morphology that we found in our more poorly-differentiated cell lines following knock down. In OC cells, the kinase Pak2 was shown to bind RP11-499E18.1, suggesting to the authors that interference with Pak2-SOX2 interaction in the cytoplasm inhibited EMT transition. The underlying hypothesis of RP11-499E18.1 mechanism of action is focused on potential chromatin-modifying effects, which is quite different than that of Yang et al, although the models are not mutually exclusive.

### Example 6: Myoblast Determination Protein (MyoD1) TEr and Muscle/Cardiovascular Genes

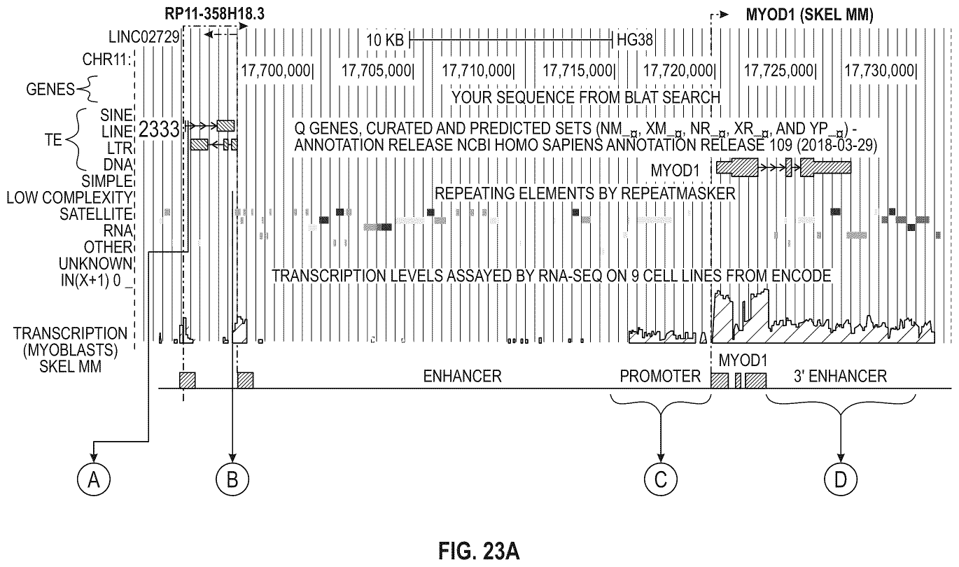

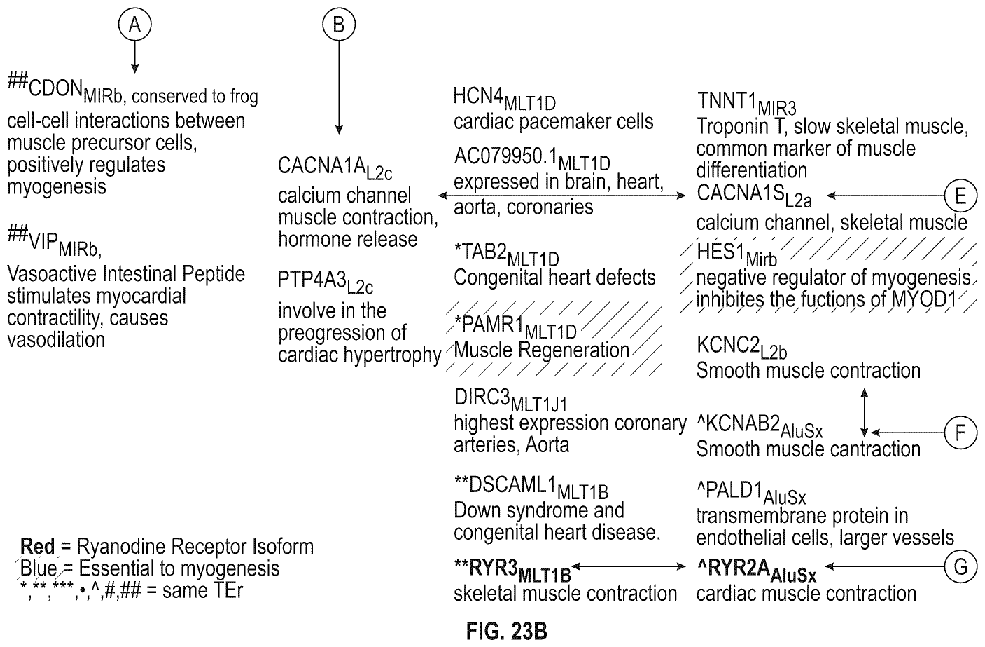

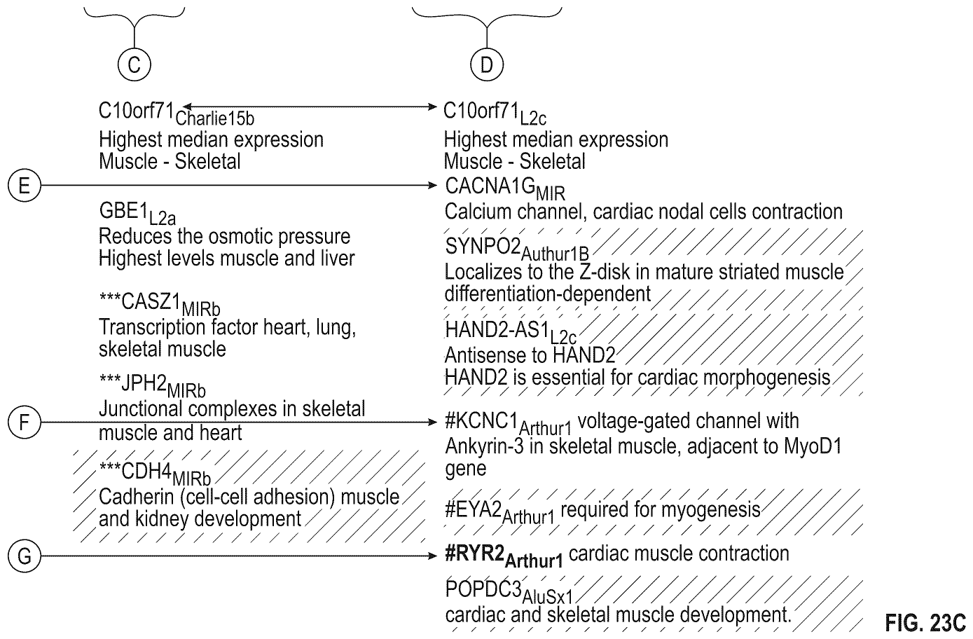

The alignment to pathway-specific genes of TEr of key genes and their cis lncRNA was further tested in detail using TEr of MyoD1 (major role in regulating muscle differentiation) and its upstream lncRNA_{RP11-358H18.3} (FIG. 23). MyoD1 promoter and 3′ enhancer contain numerous TEr than are strongly transcribed in muscle cell (myoblast) tissue culture, as is lncRNA_{RP11-358H18.3} (FIG. 23) Bioinformatics analysis of these TEr revealed a significantly high number of alignments to other genes of the muscle/cardiovascular system (P<0.00004 vs random TE; P<0.0008 vs hair gene controls; P<0.00009 vs housekeeping genes) (Table 7). An astonishing number of alignments were to genes of myogenesis, and often the same TEr would align 2 or more genes required for muscle development or maintenance (FIG. 23). For example, highly conserved MIRc in exon 2 (of 3) of lncRNA_{RP11-358H18.3} aligned with high-identity to both CDON1 (a mediator of cell-cell interactions specifically between muscle precursor cells) and to VIP (critical protein of cardiac muscle contraction and vasodilation (FIG. 23). These results suggest that TEr sequence in lncRNA participate in the trans localization of lncRNA to genes of the same pathway as those targeted by the TEr of its associated coding-gene and imply the specificity of the reaction is due to lncRNA nucleotide sequences such as exonic TEr.

### Example 7: Steroid Receptor RNA Activator 1 (SRA1) TEr and Genes Associated with Parkinson's Disease

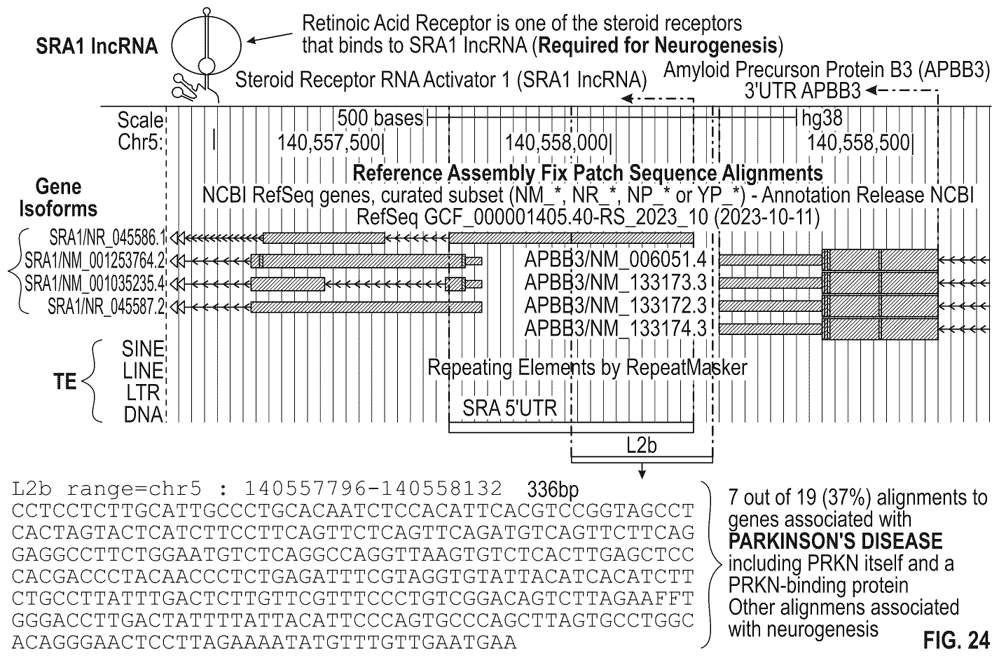

In contrast to protein coding genes, 83% of lncRNAs contain a TE, and TEs comprise 42% of lncRNA sequences. (Kapusta A, Kronenberg Z, Lynch V J, Zhuo X, Ramsay L A, Bourque G, et al. Transposable Elements Are Major Contributors to the Origin, Diversification, and Regulation of Vertebrate Long Noncoding RNAs. PLoS Genetics. 2013; Alfeghaly C, Sanchez A, Rouget R, Thuillier Q, Igel-Bourguignon V, Marchand V, et al. Implication of repeat insertion domains in the trans-activity of the long non-coding RNA ANRIL. Nucleic Acids Research. 2021; 49(9):4954-70.) SRA1 is a lncRNA that scaffold's hormone receptors such as Retinoic Acid Receptor (required for neurogenesis). Transcription is initiated from a L2b that forms the first half of exon 1 (FIG. 24). Surprisingly, this L2 fragment had a high likelihood of aligning genes associated with Parkinson's Disease (Table 10). Parkinson's Disease (PD) is a disorder that affects movement. The etiology of PD is unknown, although multiple genes and proteins have been identified at abnormal levels in diseased tissue. These results suggest a new model of PD pathogenesis based on aberrant transcriptional network signaling, rather than malfunction of a single gene or protein.

TABLE 10
Genes associated with Parkinson's Disease aligned by the L2-TEr sequence
initiating SRA1 lncRNA

| | Aligned | |
|---|---|---|
| | Gene | Function |
| 1 | STX18-AS1 | antisense to Syntaxin (depolarization of the presynaptic axonal boutons). |
| | | STX1B and STX6 are associated with PD |
| 2 | PRKN | Parkinson Protein 2, E3 Ubiquitin Protein Ligase, targets substrate proteins for |
| | | proteasomal degradation, first gene identified as associated with PD |
| 3 | FILIP1 | filamin A interacting protein 1,. promotes filamin A degradation; role in cortical |
| | | neuron migration and dendritic spine morphology, associated with PD |
| 4 | PLA2G2C | Secretory Phospholipase A2 Group IIC. Secretory PLA2 is involved in LPA production |
| | (aligned | (lysophosphatidic acid; involved in neural development; activates microglial cells). |
| | again by | PLA2-G6 is associated with adult-onset dystonia-parkinsonism; PLA2G1B and PLA2G10 |
| | SRA1 | are differentially expressed in the substantia nigra of PD patients |
| | intron 2 | |
| | L2a | |
| 5 | OTUD3 | deubiquitinase OTUB1 is amyloidogenic, neurotoxic and forms inclusions with |
| | | α-synuclein (Lewy bodies) in rotenone-induced mouse model of PD |
| 6 | SYT6 | synaptotagmin 6. Highest median expression in basal ganglia, Ca2+ dependent |
| | | exocytosis of vesicles. Synaptotagmin interacts directly with PRKN |
| 7 | IGSF21 | immunoglobin superfamily member 21, synaptic inhibition through interactions with |
| | | NRXN2 (neuronal cell adhesion molecule down regulated in PD) |

### Example 8: NFkB1 Promoter Non-Processive “Junk” Transcripts and Genes Participating in Formation, Processing, Packaging and Function of mRNA

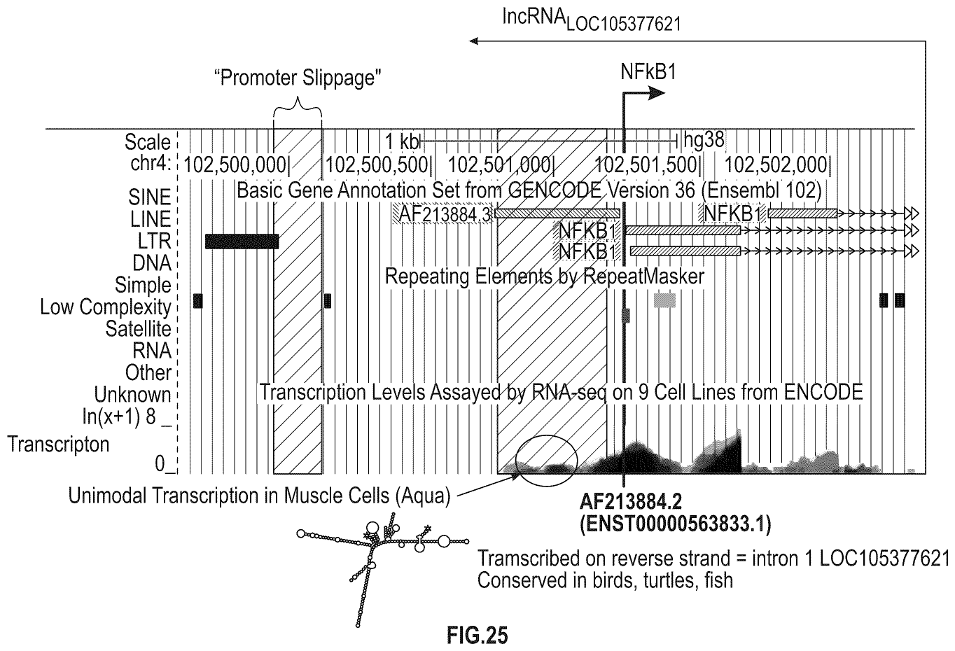

TEr are not the only “junk” found at the promoter. Bidirectional promoter transcripts are often considered “Promoter Slippage”. Although nascent RNA sequencing reveals widespread pausing and divergent initiation at human promoters, a function for these nonprocessive transcripts (NPtx) is unknown (FIG. 25). (Core L J, Waterfall J J, Lis J T. Nascent RNA sequencing reveals widespread pausing and divergent initiation at human promoters. Science. 2008.) The in silico method indicated that there is a significant likelihood that NFkB1 “promoter slippage” NPtx and lncRNA AF213884.2 share high-identity TEr within genes encoding RNA-binding proteins participating in formation, processing, packaging and function of mRNA (Table 11).

The presence of these conserved and transcribed “promoter slippage” sequences within the promoter of NFkB1 suggest that, 1) Transcription Factors are not always bound to active promoter regions, allowing antisense transcription to occur; and 2) there is potential for RNA-mediated transcriptional crosstalk between the NFkB1 promoter non-TE sequences and genes that code for RNA-binding proteins critical to RNA elongation and transport.

TABLE 11
Significant likelihood that NFKB1 promoter slippage NPtx
and IncRNA AF213884.2 share high-identity
TEr within RNA-binding protein genes

| AF213884.2 (479bp) | | | | | Promoter Slippage NPtx | | | | |
|---|---|---|---|---|---|---|---|---|---|
| Aligned | Gene | Region | Summary | Definition | Aligned | Gene | Region | Summary | Definition |
| 1* | TMEFF2 | 5′UT [symbol] | ERK1/2 | Transmembrane Protein | 1 | NFkB1 | Pro | NFkB | NFkB1 promoter |
| | | | phos- | With EGF Like And Two | | NPtx | | | nonprocessive |
| | | | phorylation | Follistatin Like Domains | | | | | transcripts |
| | | | | 2, Both an oncogene and | 2 | RBM15 | In 1/2 | RNA | RNA Binding |
| | | | | a tumor suppressor, | | | | binding | Motif Protein |
| | | | | proteolytic shedding | | | | | 15, mediates N6- |
| | | | | induced by TNFa promotes | | | | | methyladenosine |
| | | | | ERK1/2 phosphorylation | | | | | (m6A) methylation |
| 2* | hnRNP-L | 5′UT [symbol] | RNA | Heterogeneous nuclear | | | | | of RNAs, a |
| | | | binding | ribonucleoprotein L, | | | | | modification that |
| | | | | stably associated with | | | | | plays a role in the |
| | | | | hnRNP complexes, a major | | | | | efficiency of |
| | | | | role in the formation, | | | | | mRNA splicing |
| | | | | packaging, processing, | | | | | and RNA |
| | | | | and function of mRNA | | | | | processing |
| 3 | CFC1 | In 3/4 | patterning | Cripto, FAL-1, cryptic | | | | | |
| | | | the left-right | family 1, involved | 3 | AC022634.2 | Enh | unknown | [symbol] ncRNA |
| | | | embryonic | in signaling during | | | | function | |
| | | | axis | embryonic development | 4 | RPL3 | Pro | RNA | Ribosomal |
| 4* | Intragenic | | Transposable | Tigger6a range chr11: | | | | binding | Protein L3, HIV-1 |
| | | | Element | 127658493-127658843 | | | | | TAR RNA- |
| 5 | MEF2C- | In 5/5 | Transcription | Antisense to Myocyte | | | | | Binding Protein B |
| | AS1 | | Factor for | Enhancer 2C MEF2C, | 5 | VTRNA3- | Pro | Vault | Vault RNA 3-1 |
| | | | muscle | role in maintaining the | | 1P | | RNA | Pseudogene, vault |
| | | | differentiation | differentiated state of | | | | associaed | RNAs are |
| | | | | muscle cells | | | | with | polymerase |
| 6 | LOC10798 | In 2/3 | unknown | IncRNA | | | | ribo- | III transcripts |
| | 4606 | | function | | | | | nucleo- | associated with |
| 7* | ATF7IP | 5′UT [symbol] | couples' | Activating transcription | | | | proteins | ribonucleoproteins |
| | | | transcriptional | factor 7 interacting | | | | | involved in |
| | | | factors to | protein, modulates | | | | | nucleocytoplasmic |
| | | | general | transcription regulation | | | | | transport processes |
| | | | transcription | and chromatin | 6 | BIRC3 | In 1/2 | NFKB | Baculoviral IAP |
| | | | apparatus | formation | | | | signaling | Repeat Containing |
| | | | | | | | | | 3, E3 |
| 8* | ENST0000 | 5′UT [symbol] | Unknown | Unprocessed pseudogene | | | | | ubiquitin-protein |
| | 0568980.1 | | | | | | | | ligase regulating |
| | | | | | | | | | NF-kappa-B |
| | | | | | | | | | signaling |
| | | | | | 7 | Intergenic | | | |

[symbol]

indicates data missing or illegible when filed

### Example 9: Hub Genes of Epithelial to Mesenchymal Transition (EMT) Align with High Frequency to Other Hub Genes of EMT

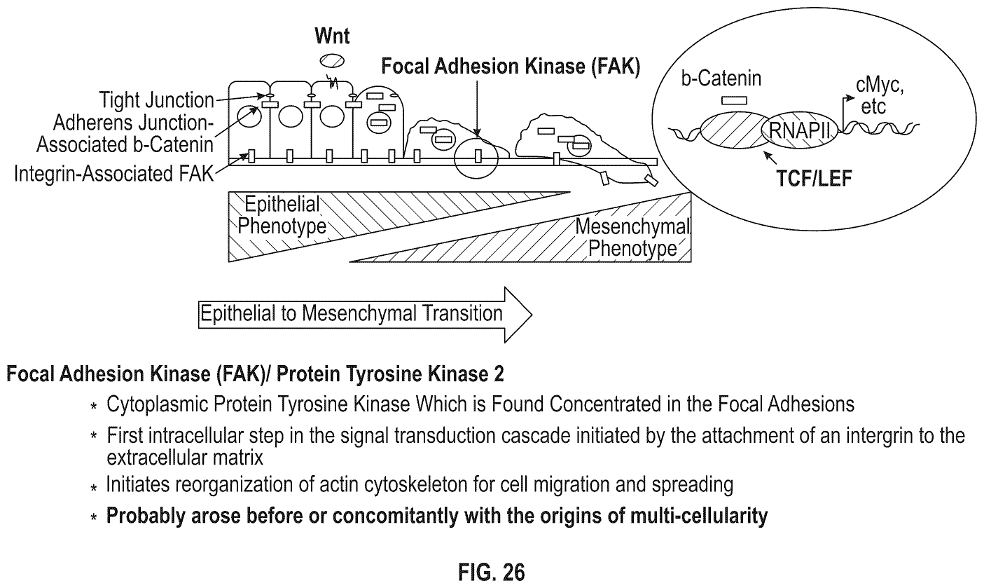

It is still unclear what specific signals induce EMT in carcinoma cells. Abnormal proliferation and apoptosis may originate from “multiple hits” within a stem cell or from signals in the tumor stroma. The canonical EMT pathway is initiated by Wnt (or Wnt/β-catenin pathway) and/or activation of Focal Adhesion Kinase (FAK, a.k.a Protein Tyrosine Kinase 2, PTK2) (FIG. 26). These proteins play an essential role in regulating cell migration, adhesion, spreading, reorganization of the actin cytoskeleton, formation and disassembly of focal adhesions and cell protrusions, cell cycle progression, cell proliferation and apoptosis. The canonical Wnt pathway triggers a cytoplasmic accumulation of b-catenin which then translocats into the nucleus where it binds directly to the TCF/LEF family of transcriptional activators (FIG. 26).

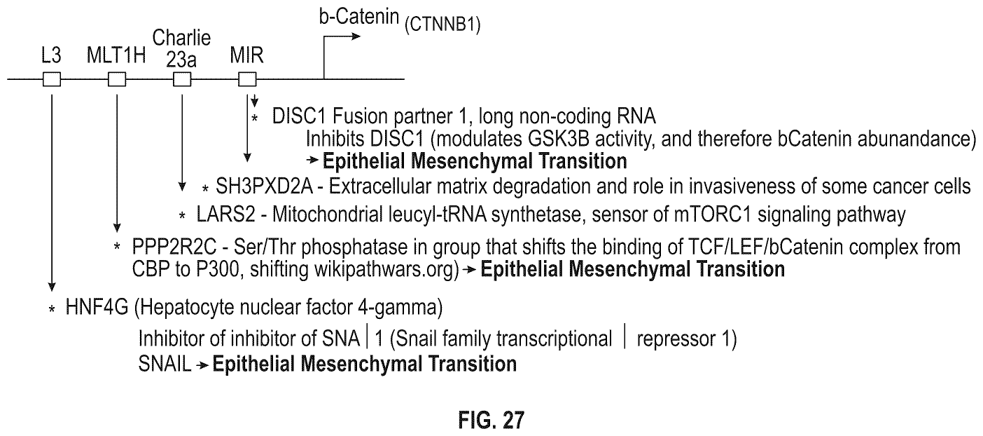

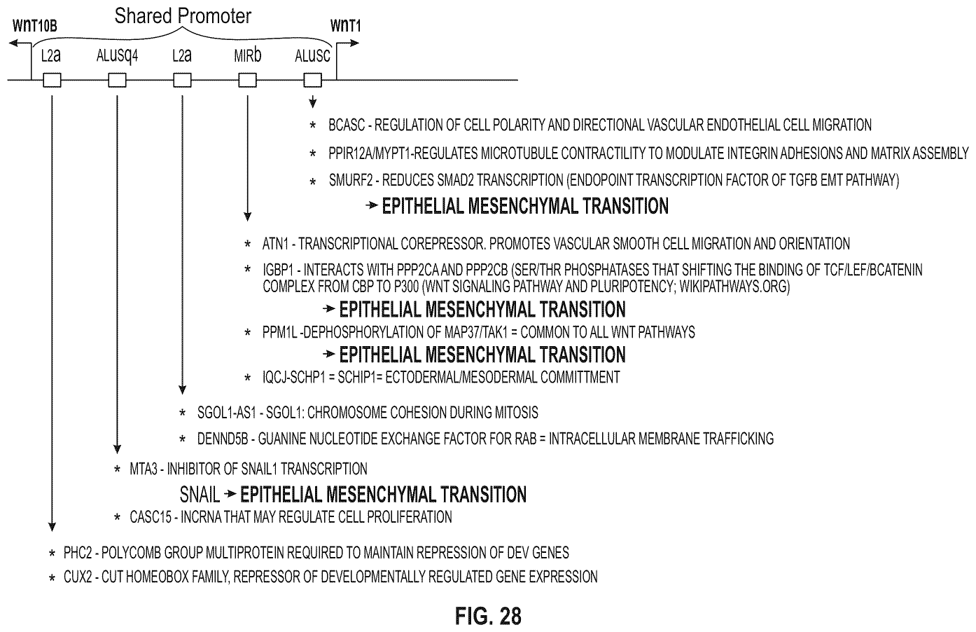

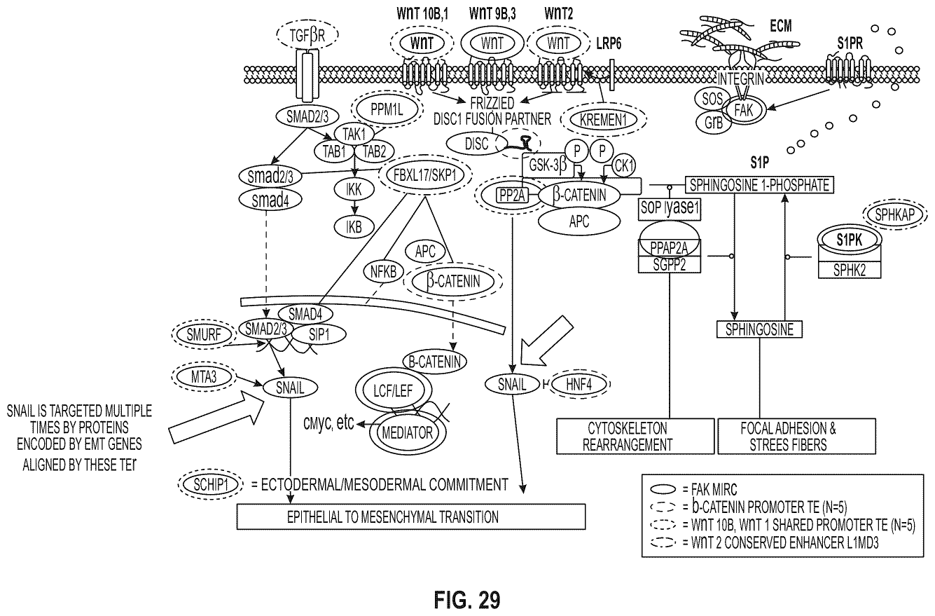

It was discovered that FAK contains a Transcription Start Site (TSS)-proximal MIRc that aligned both Wnt 3/9B and TCF7, a finding highly unlikely to be random (FIG. 26). In turn, b-Catenin itself contained promoter and TSS-proximal TEr that aligned with high sequence identities to genes required for Wnt signaling, including a lncRNA that modulates the abundance of b-Catenin itself (FIG. 27). Unlikely to be random included the finding that both b-Catenin and Wnt10B/Wnt1 promoters contained TEr that aligned Ser/Thr phosphatases shifts the binding of TCF/LEF/b-Catenin complex from CBP to P300, shifting the Wnt- signaling pathway between pluripotency and differentiation. (Wnt signaling pathway and pluripotency; wikipathways.org) (FIGS. 27, 28). In addition, critical EMT pathway genes aligned by promoter TEr of FAK, b-Catenin, Wnt10B,1 and Wnt2 participate in the regulation of SNAIL (involved in induction of the epithelial to mesenchymal transition (EMT), formation and maintenance of embryonic mesoderm, growth arrest, survival and cell migration) (FIG. 29).

### Example 10: Corticotropin Releasing Hormone Receptor 2 (CRHR2) TEr and Genes of Stress-Related Lipid Metabolism

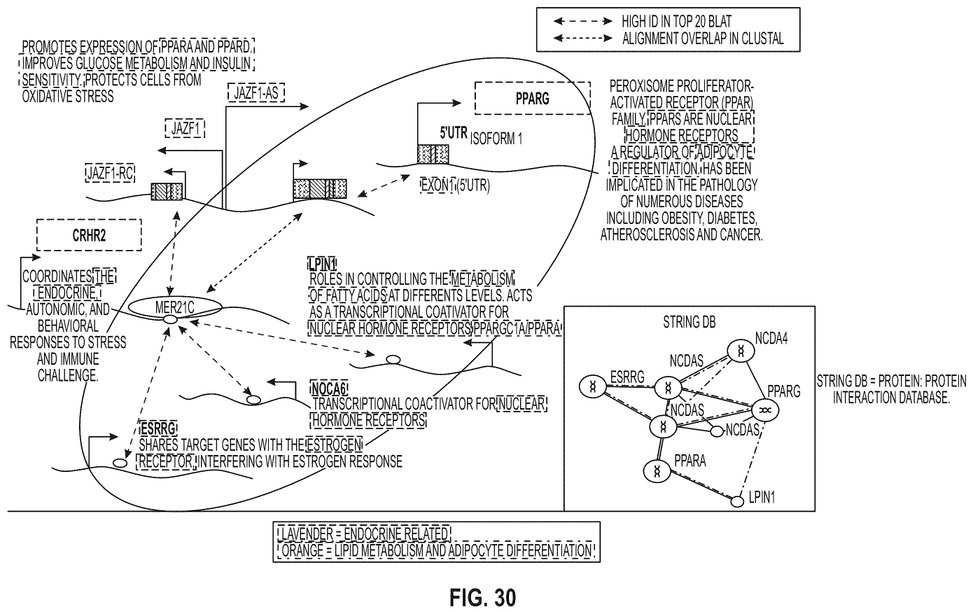

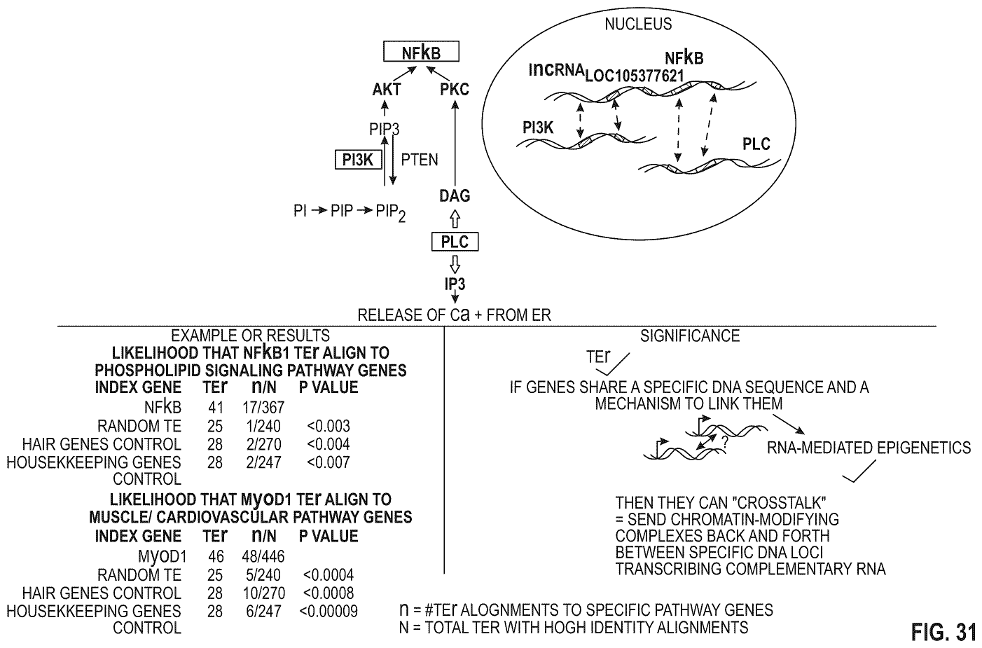

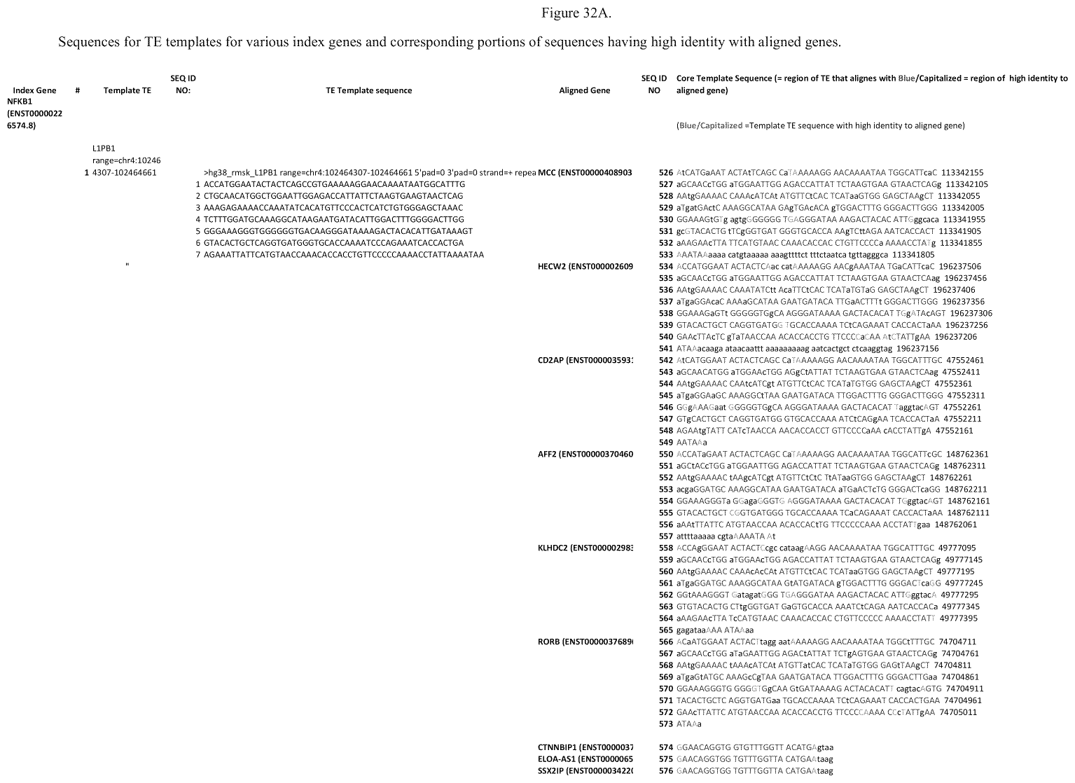

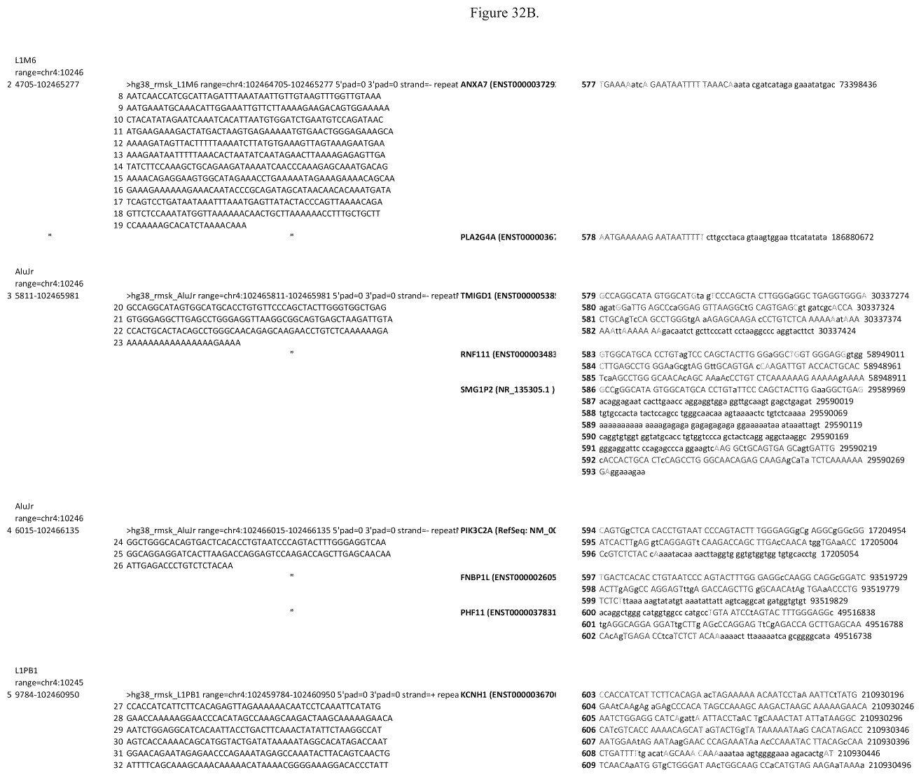

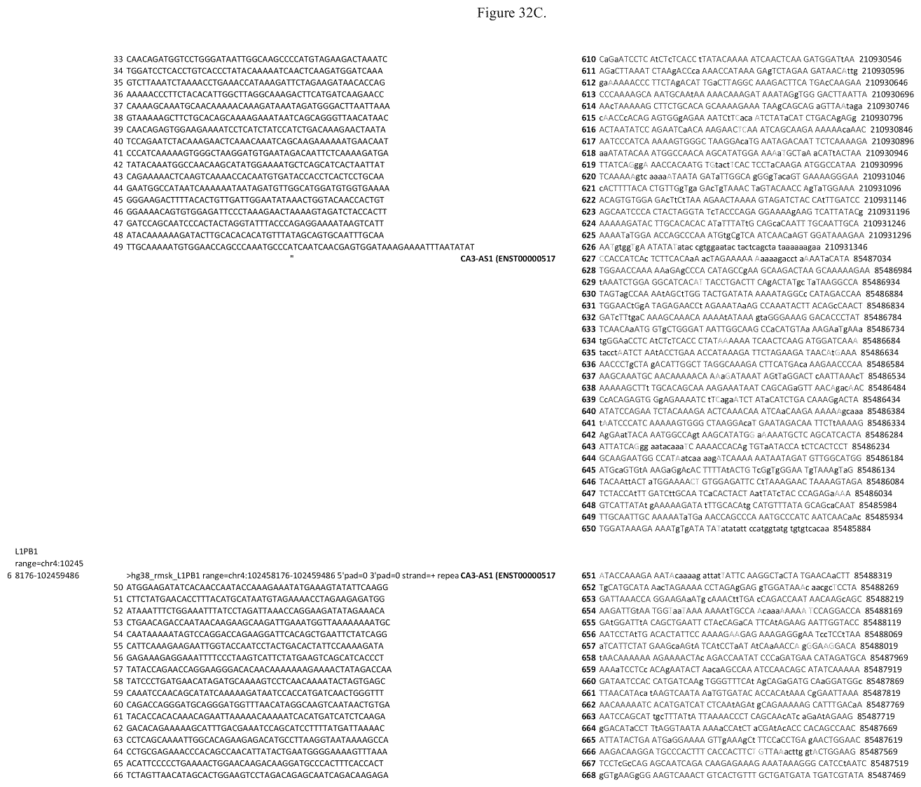

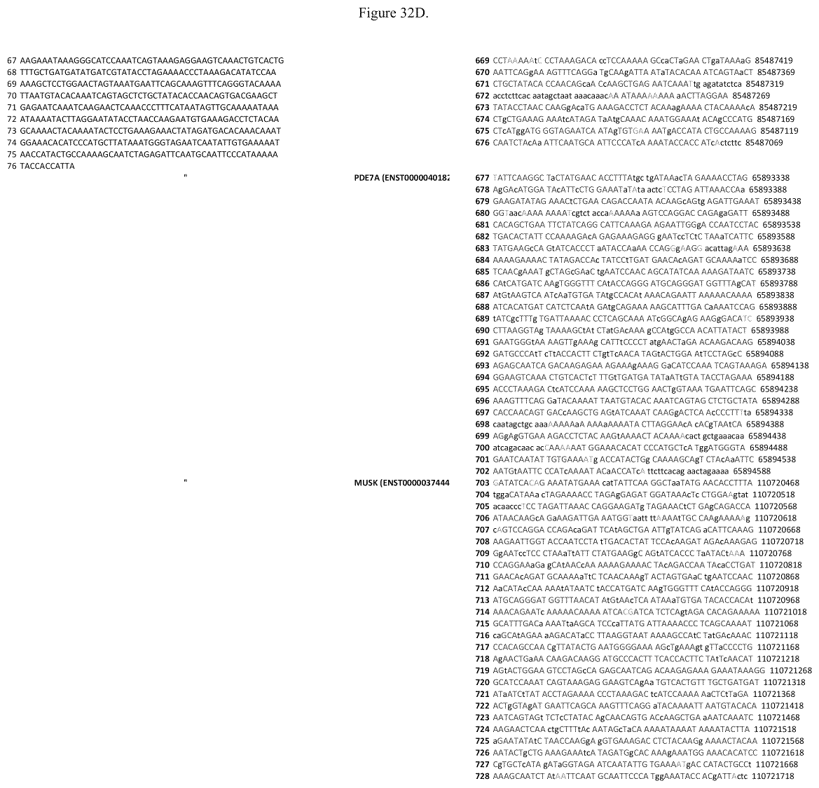

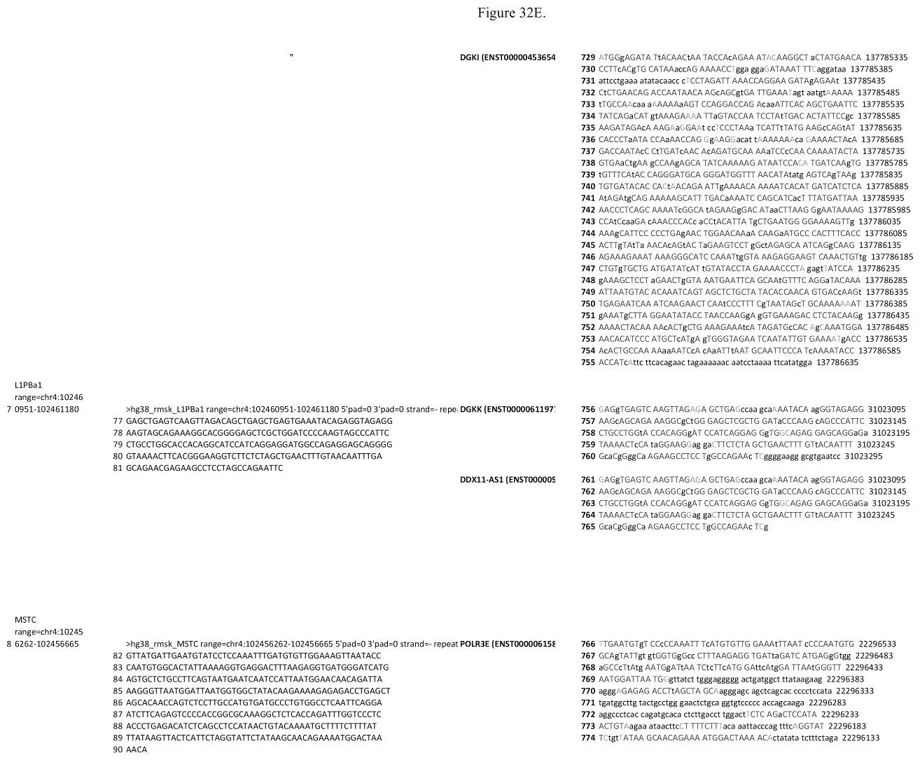

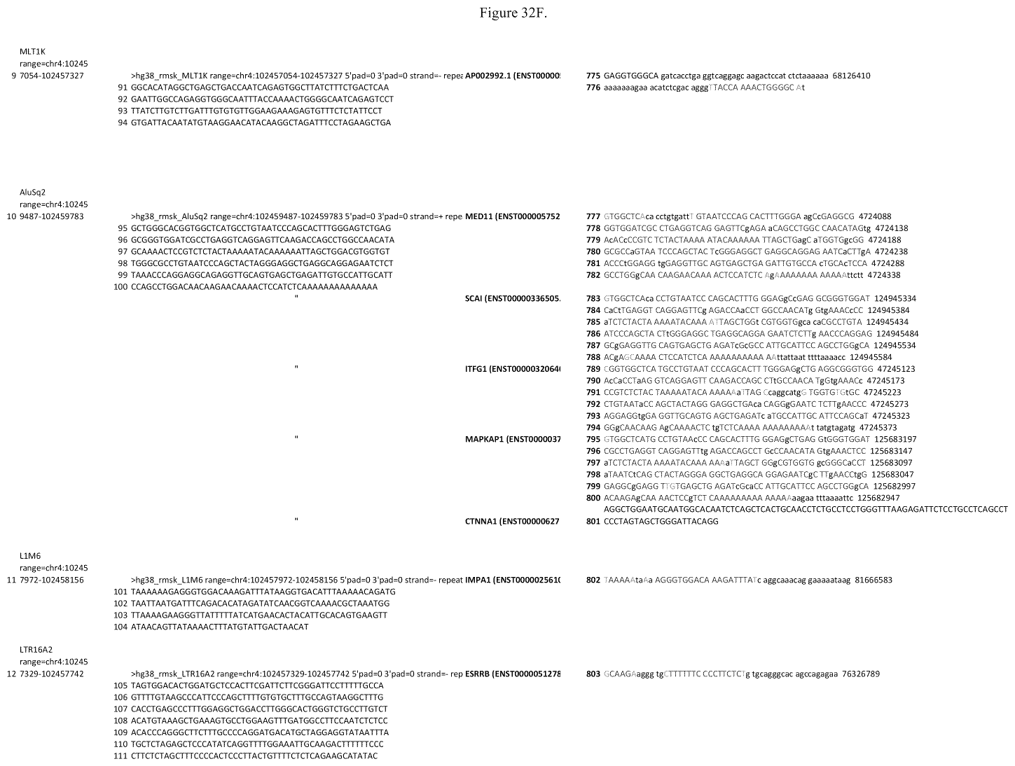

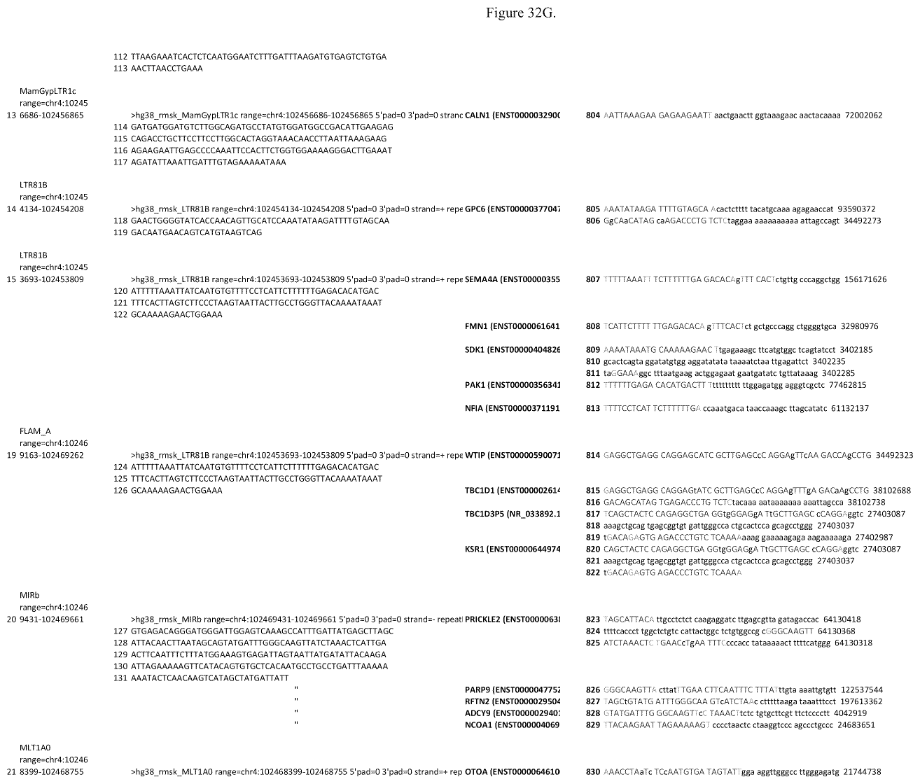

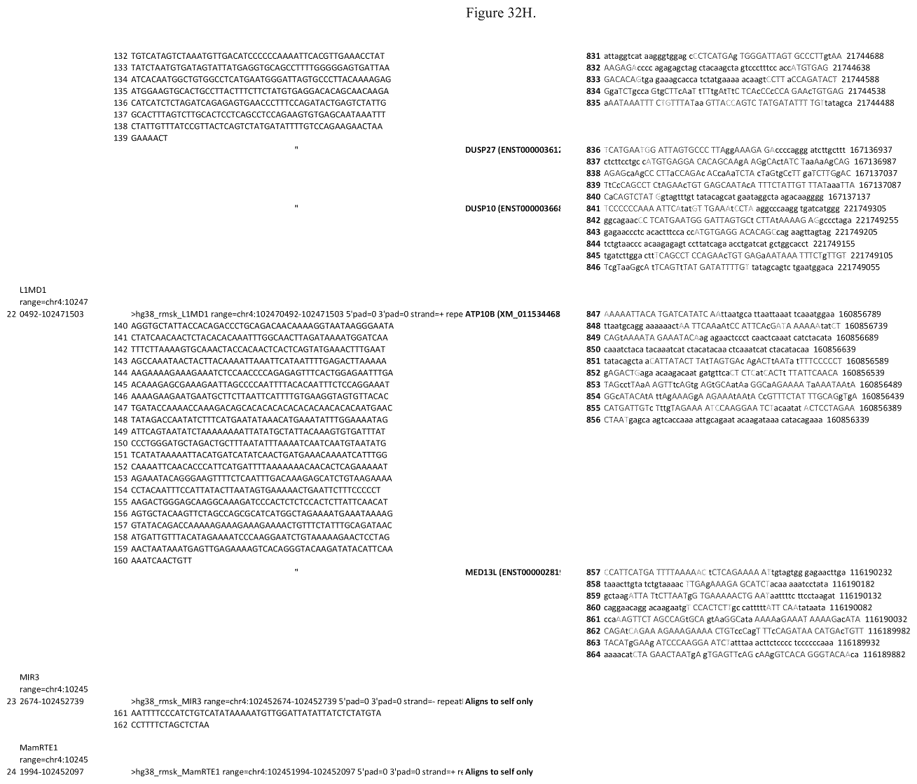

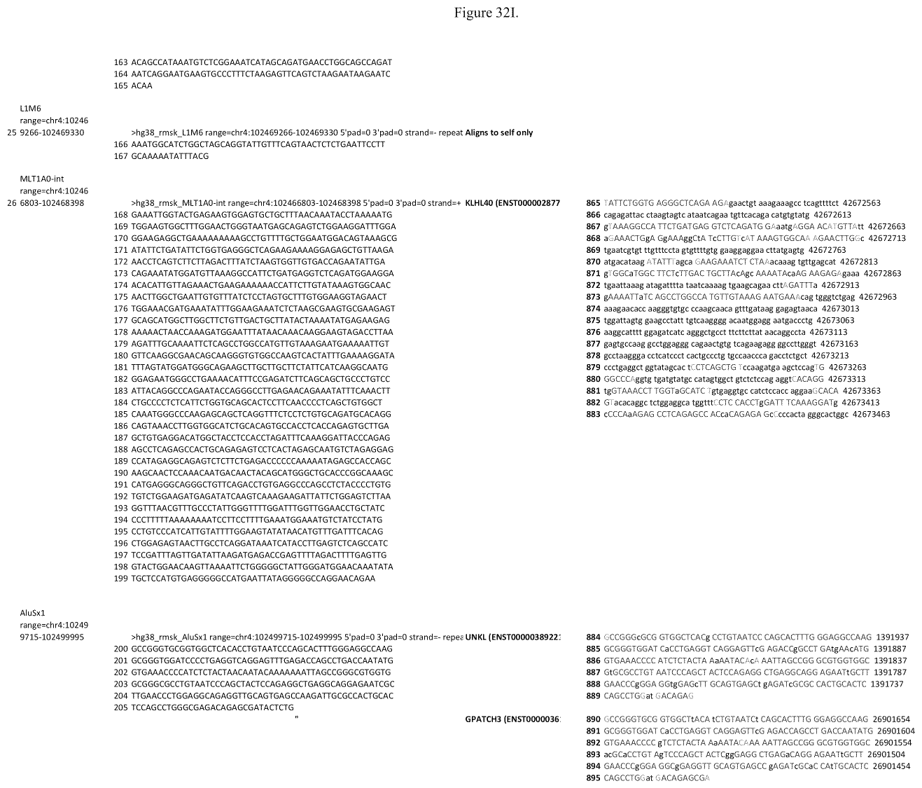

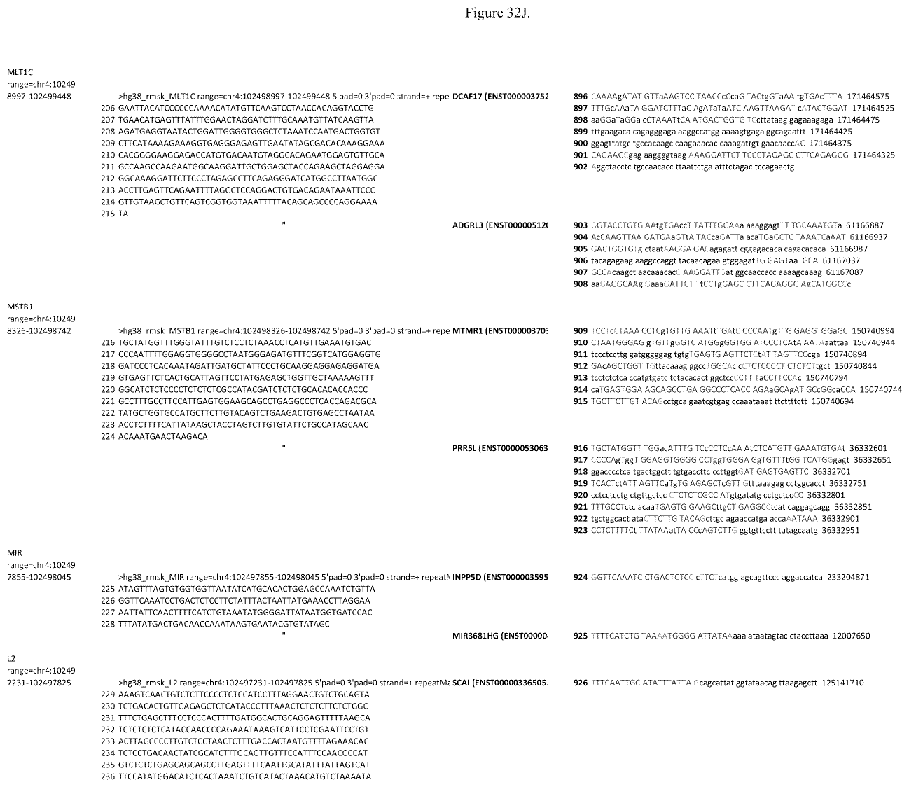

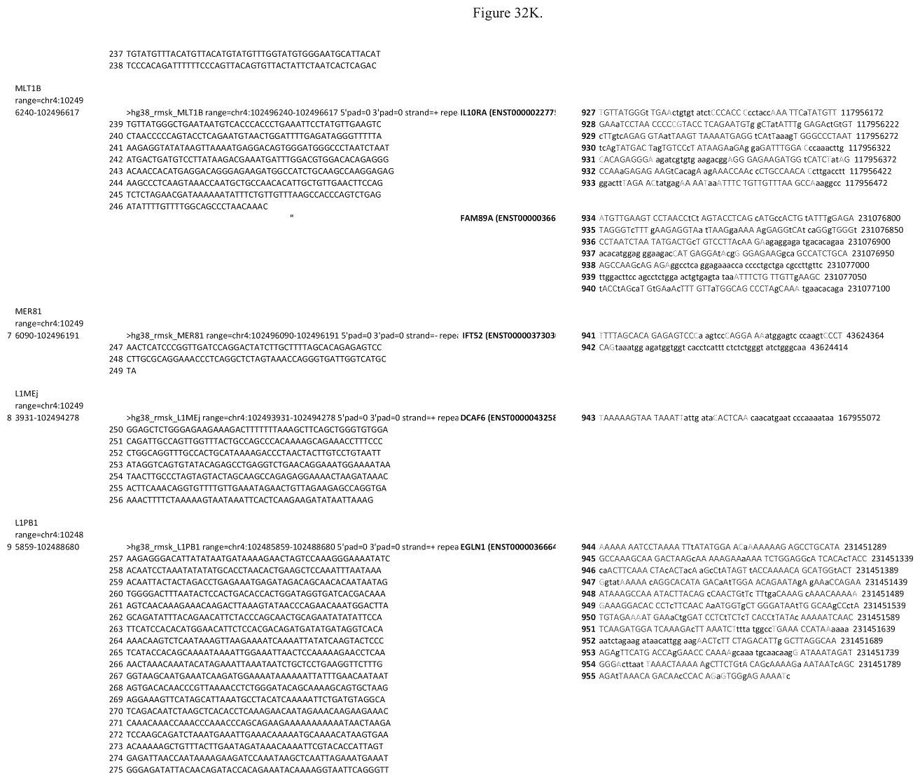

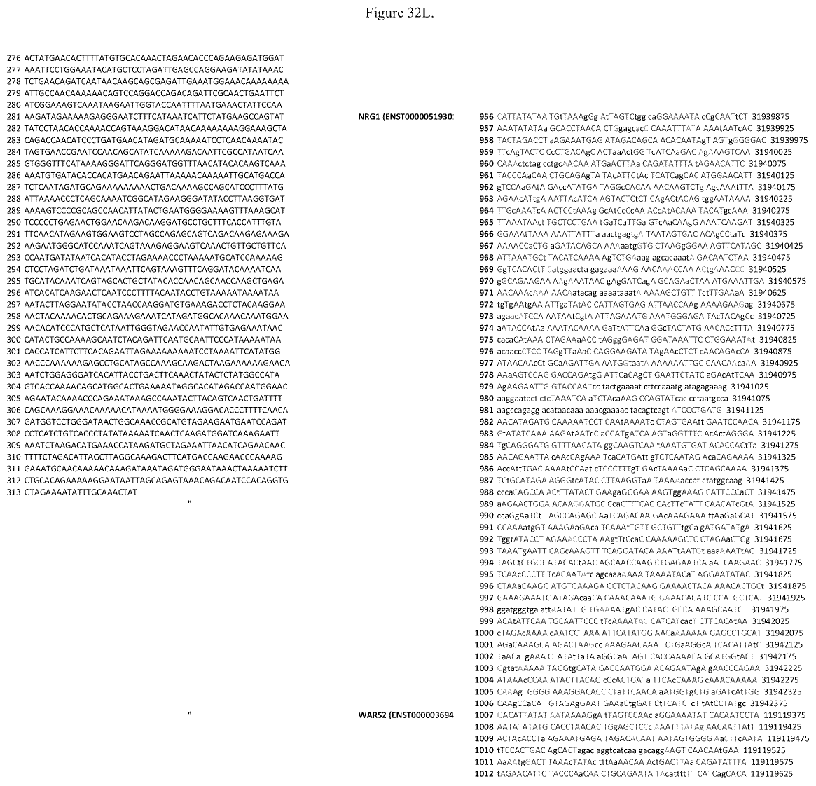

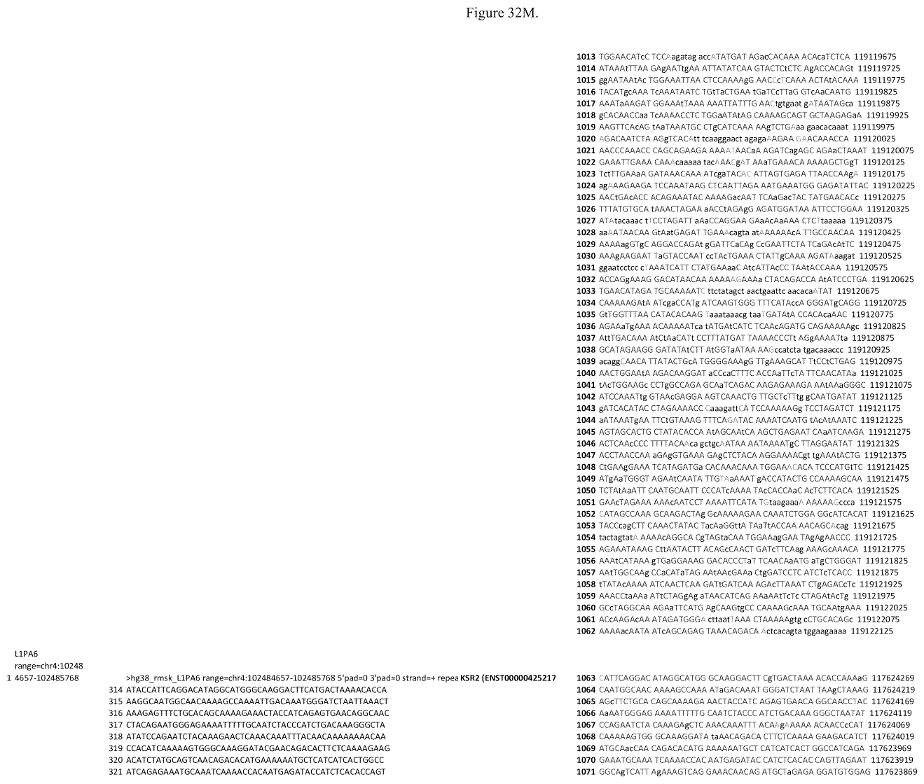

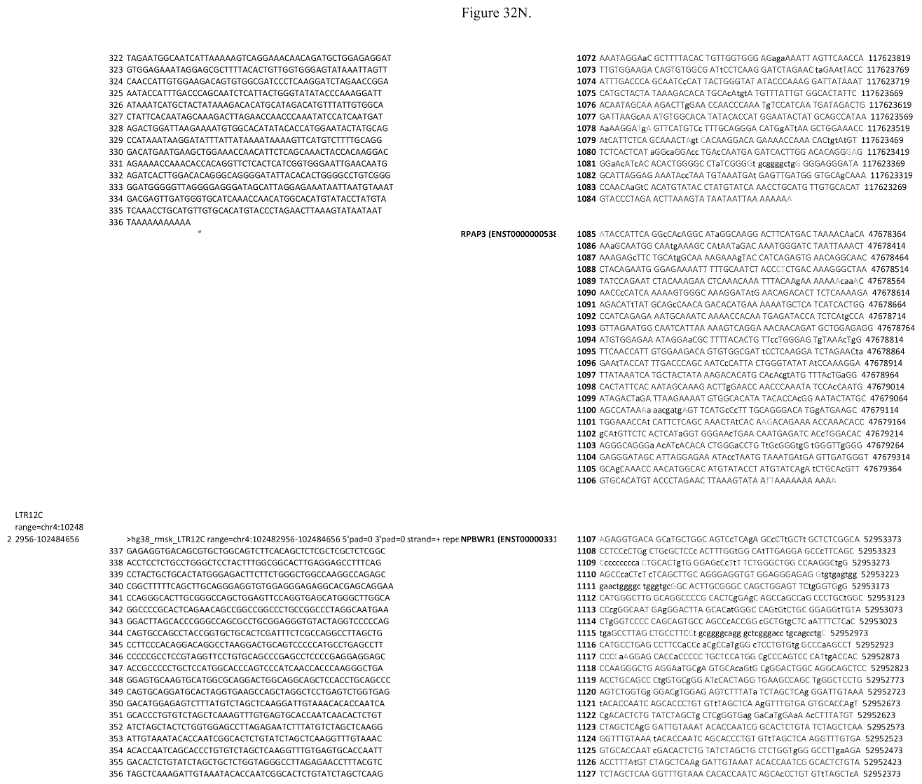

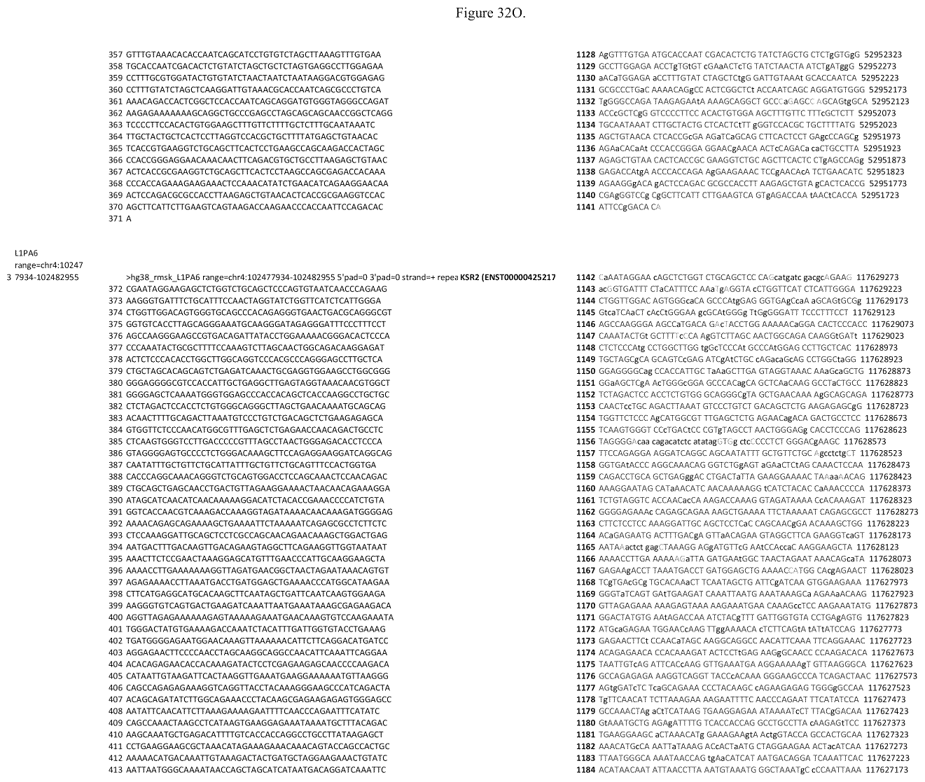

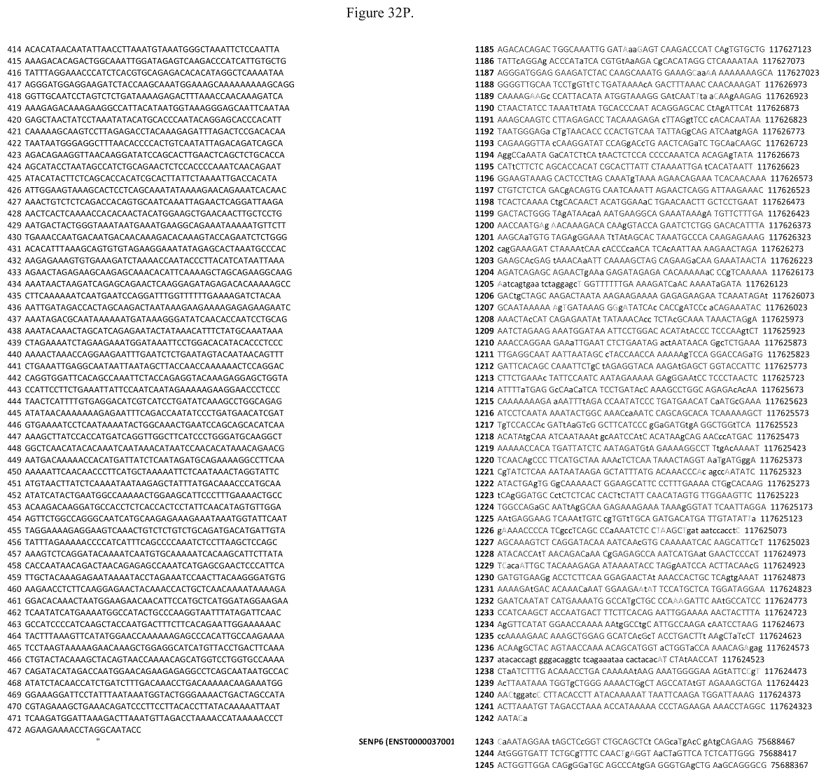

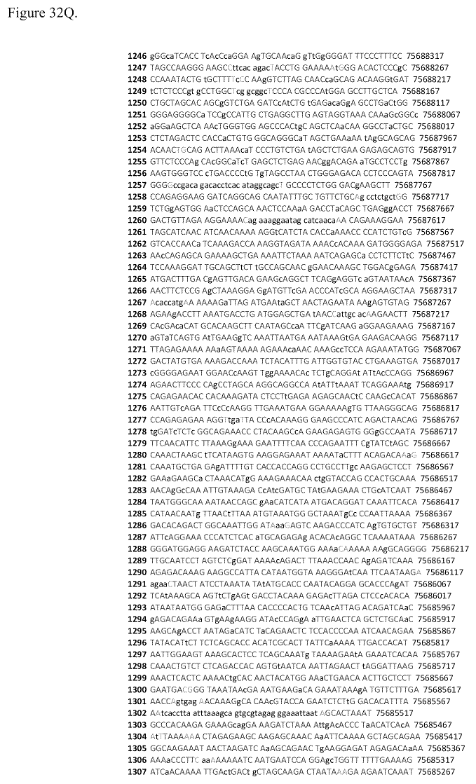

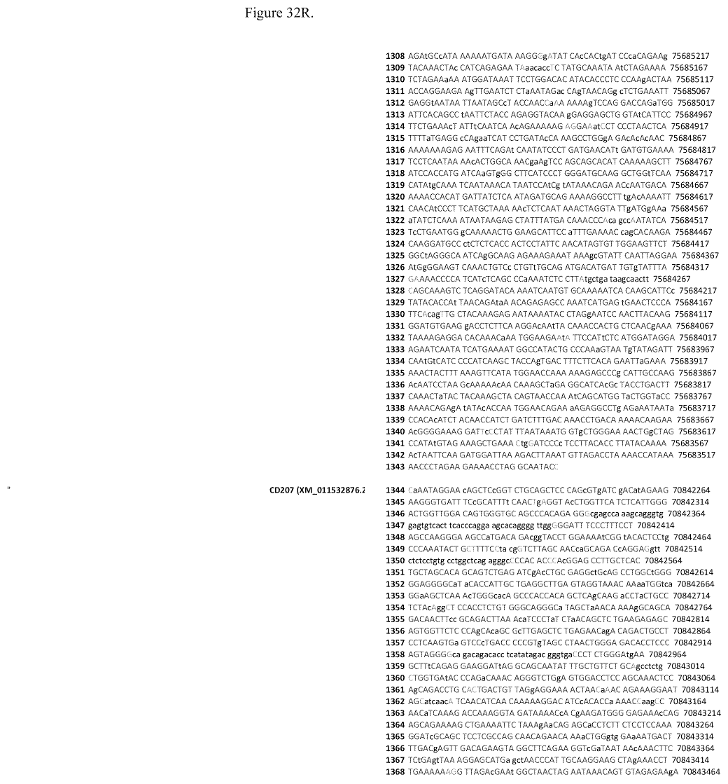

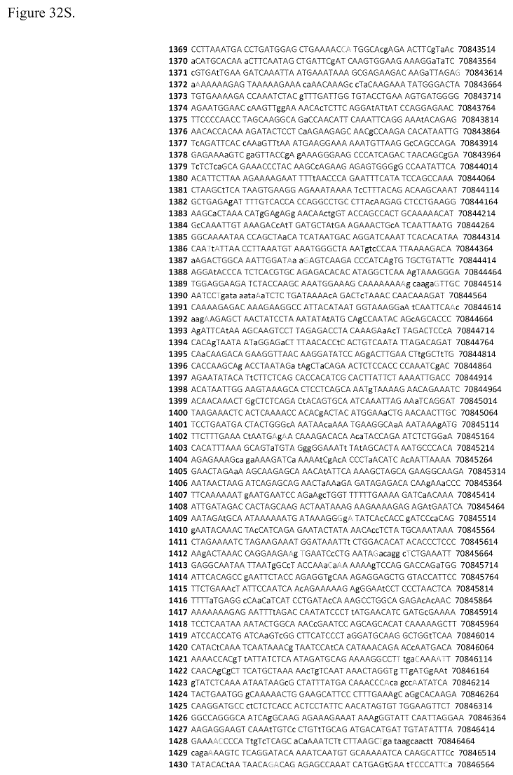

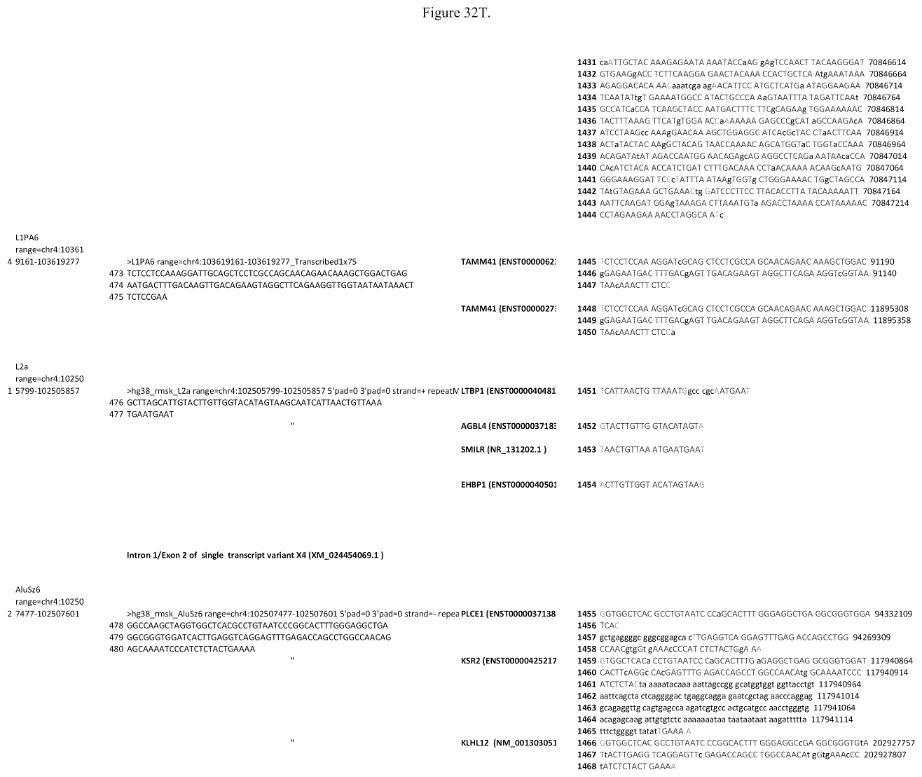

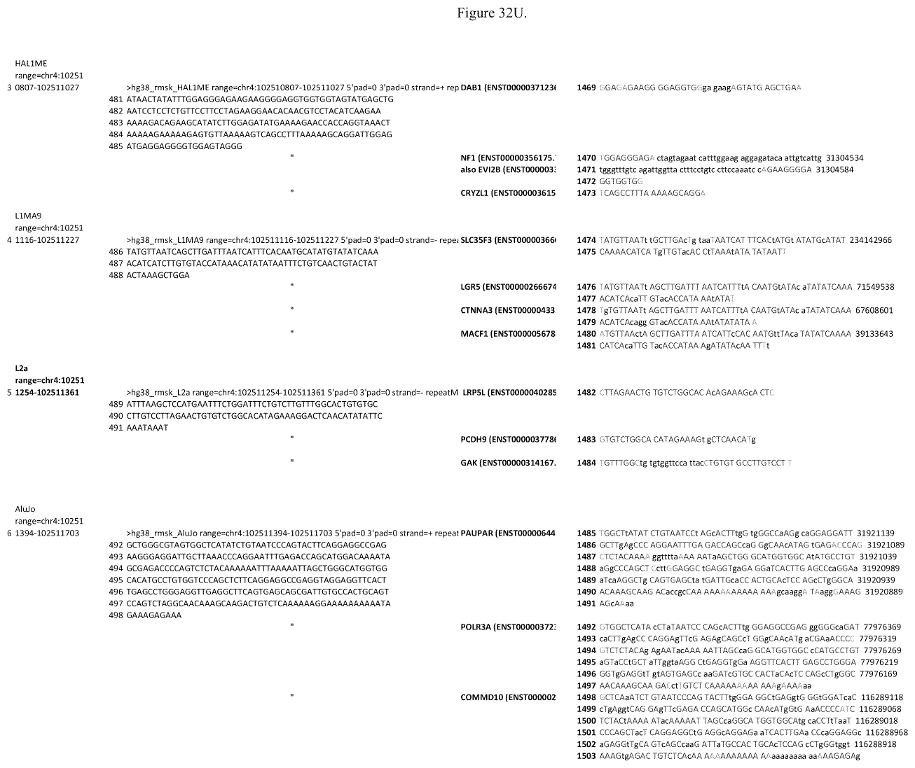

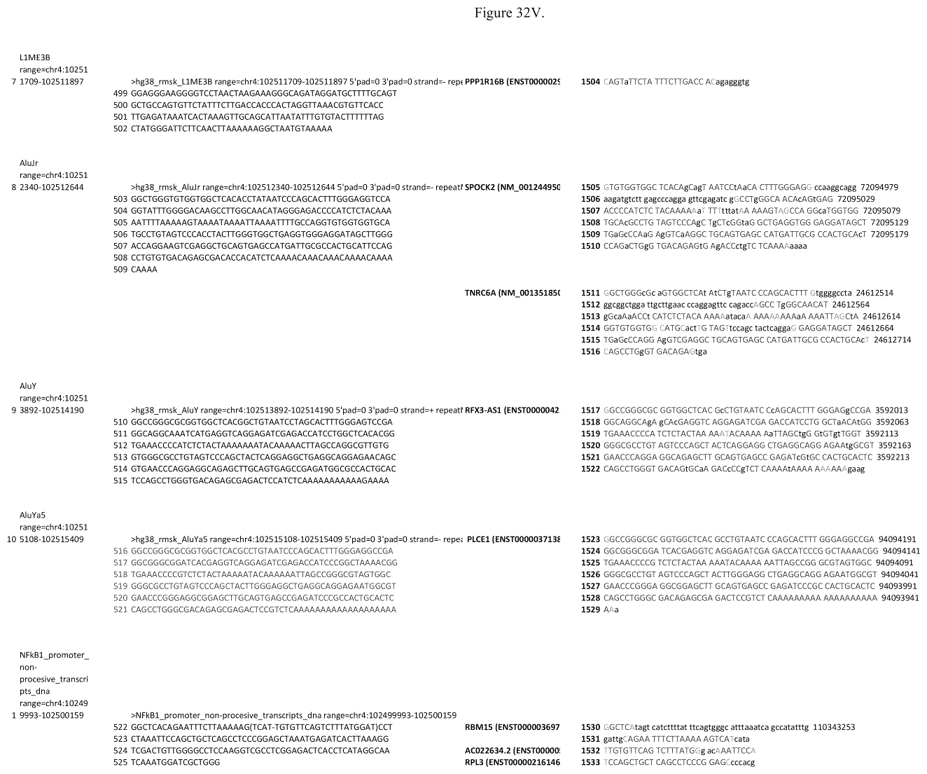

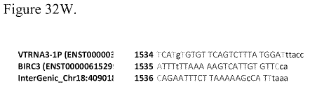

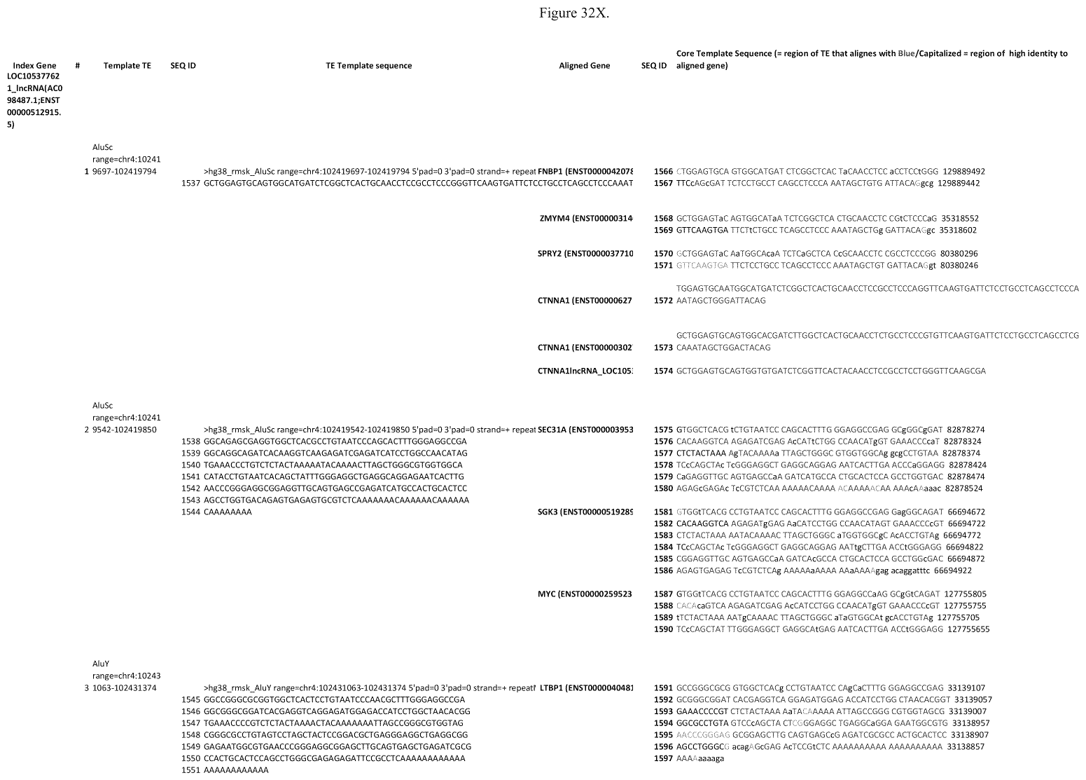

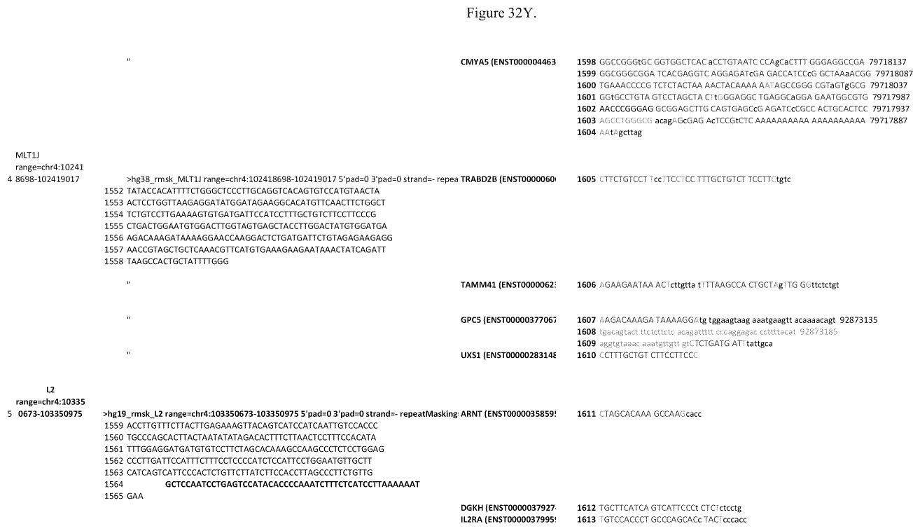

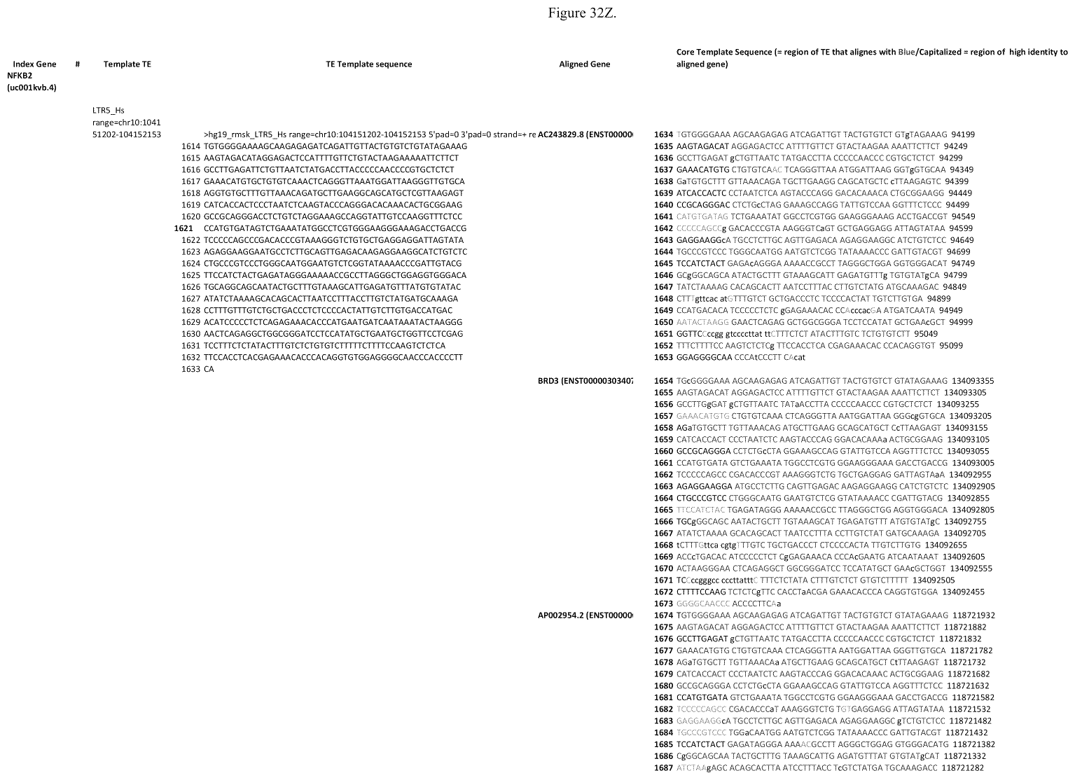

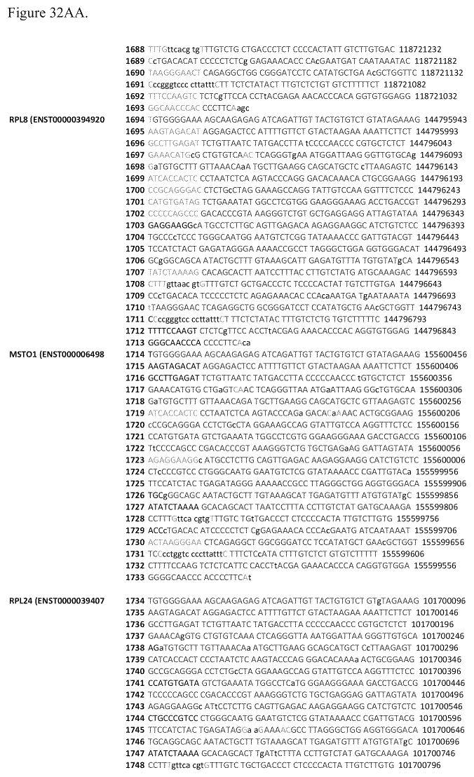

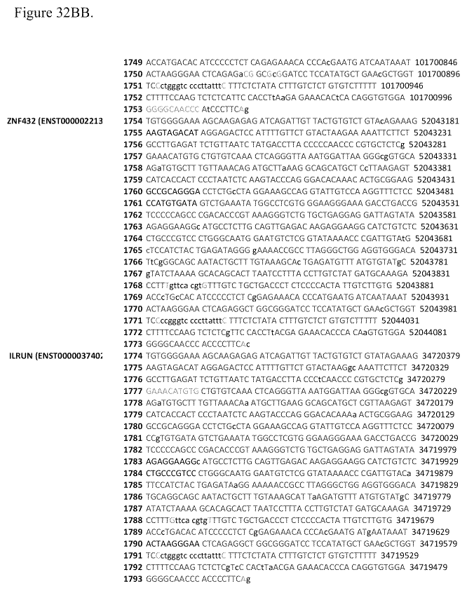

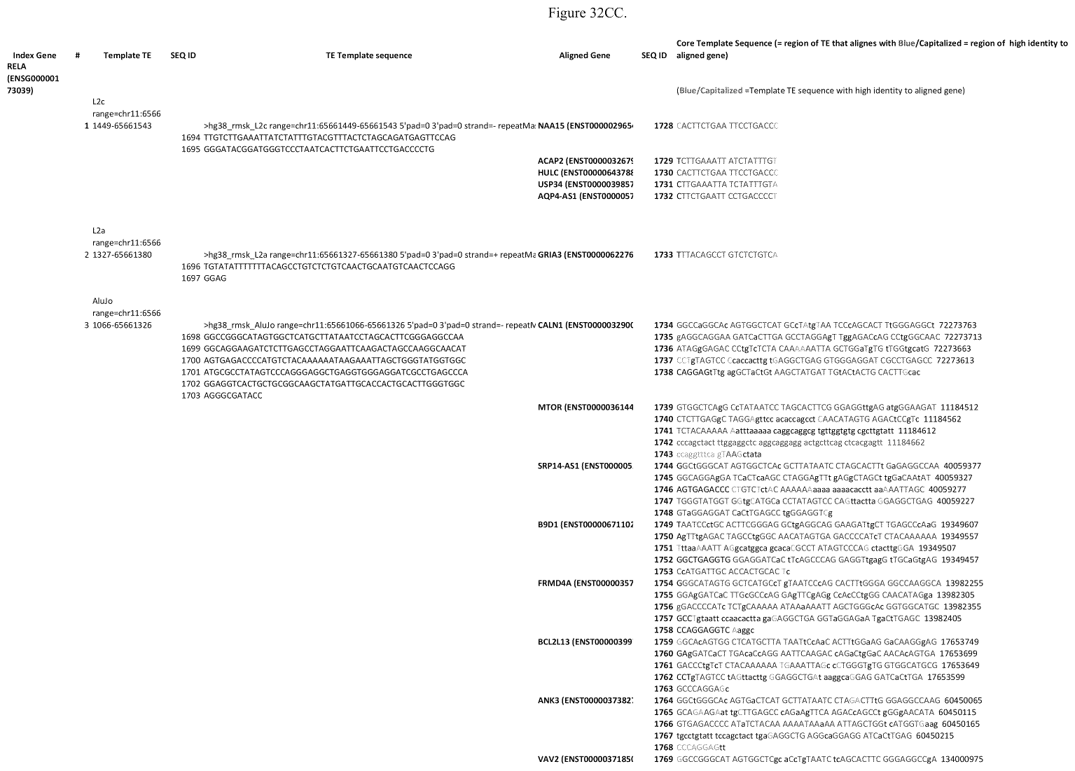

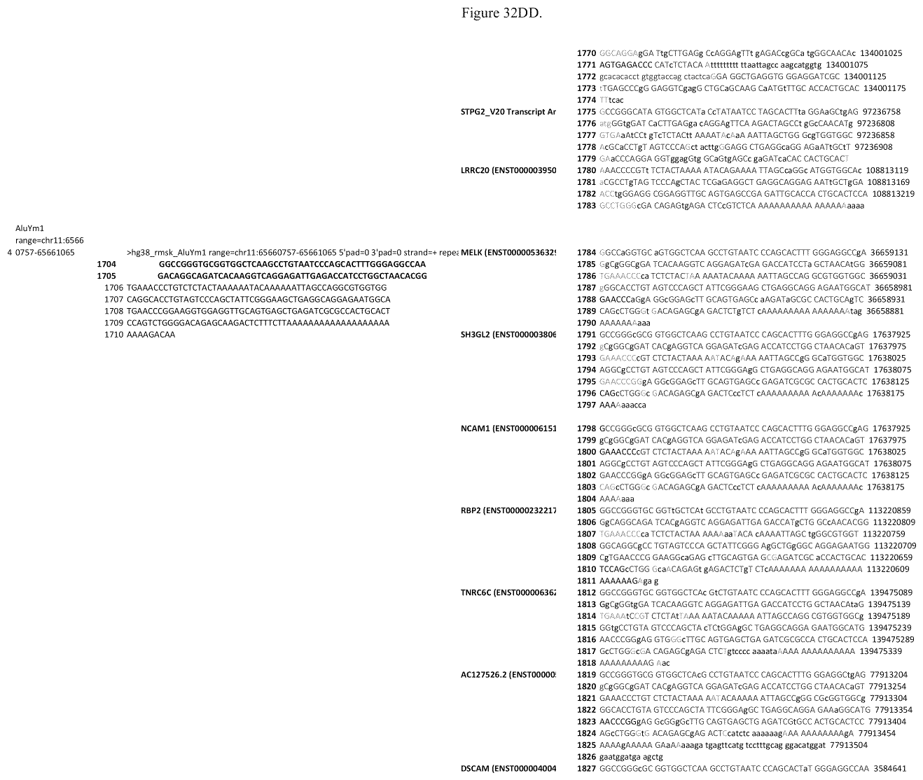

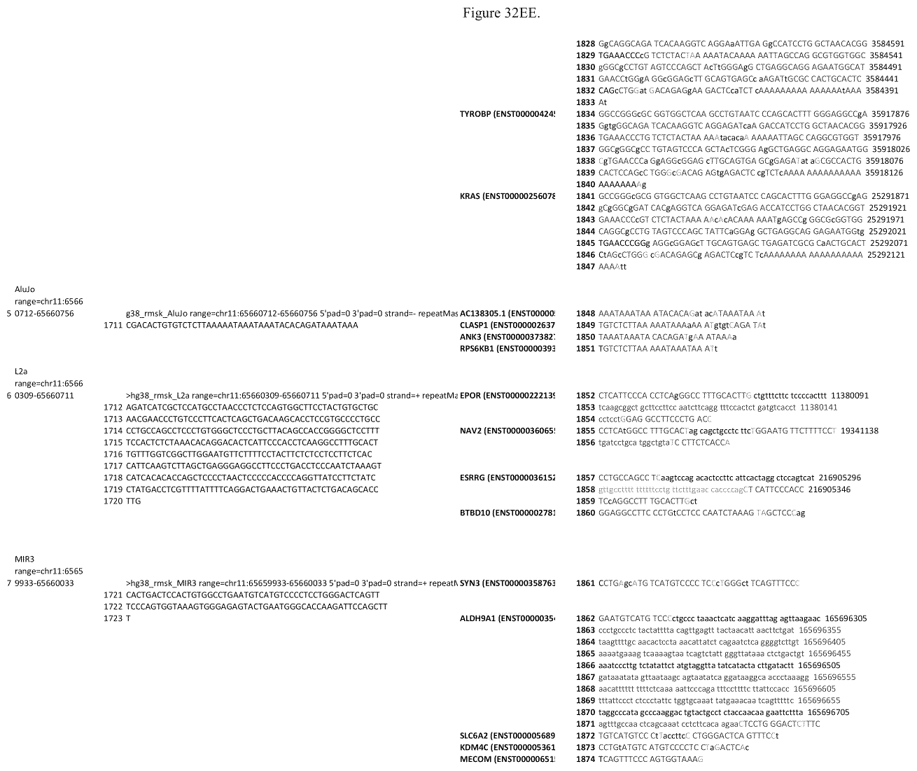

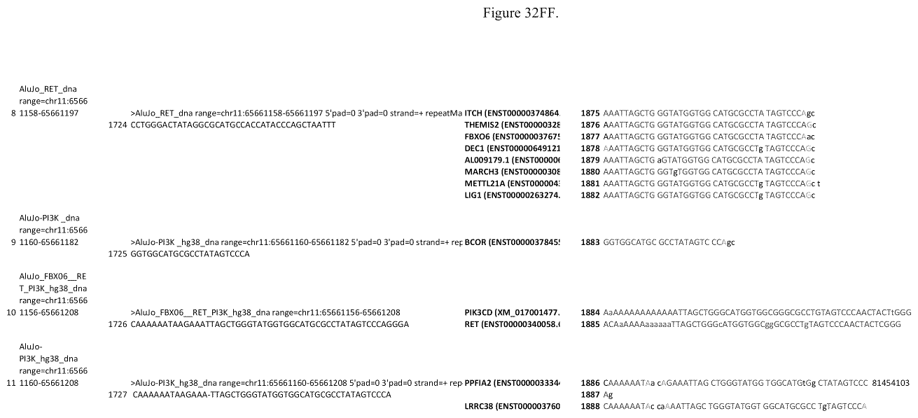

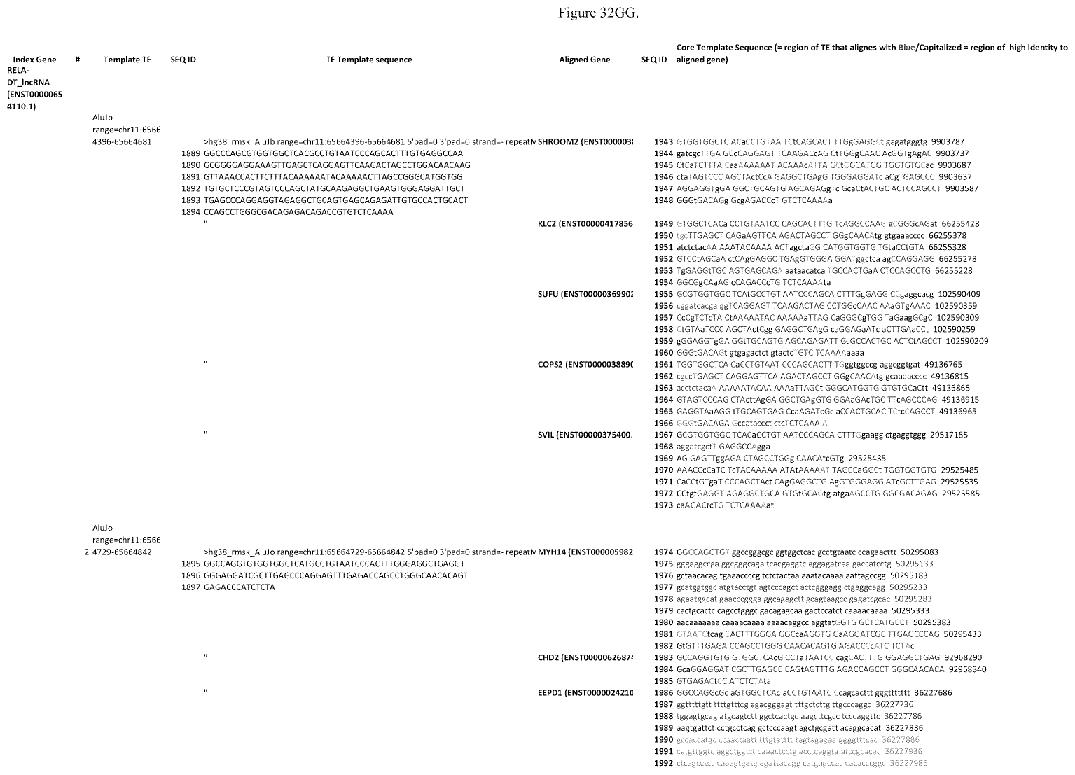

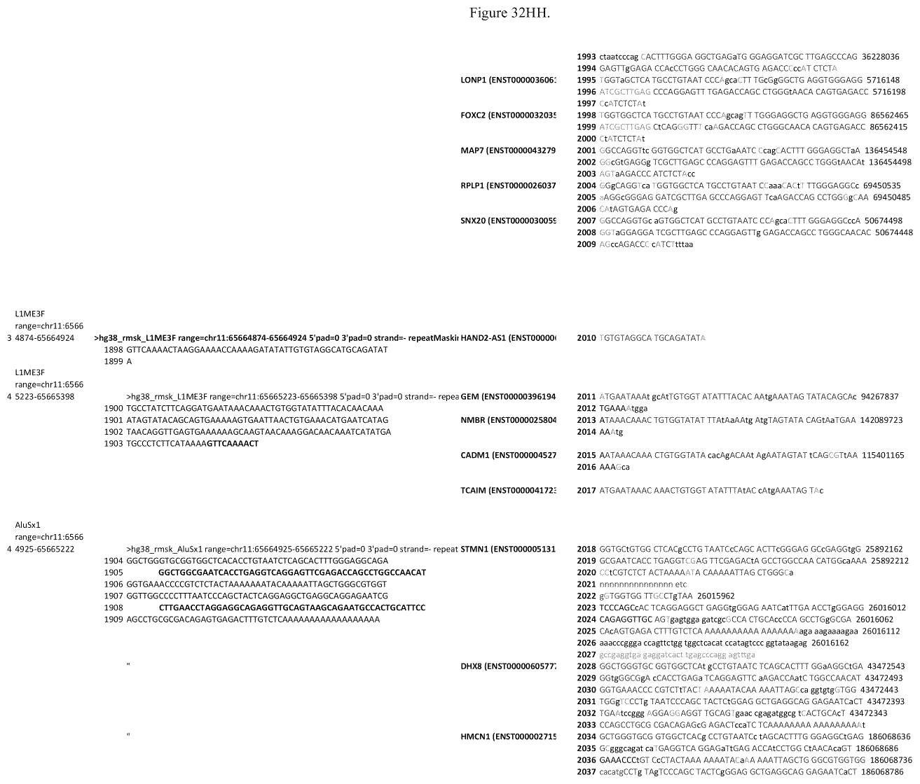

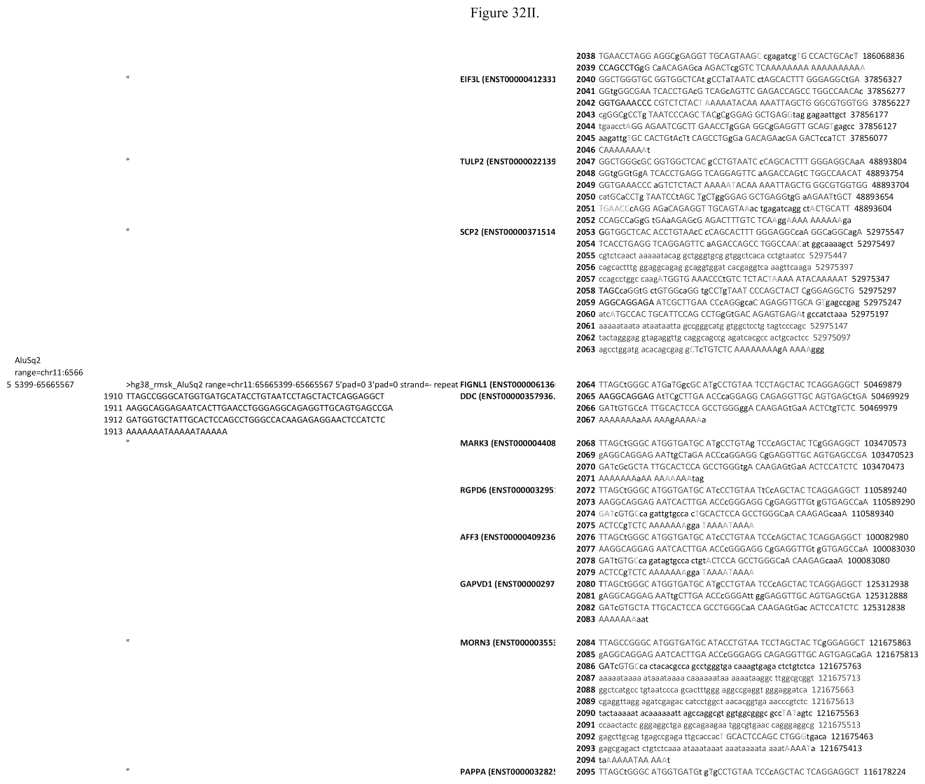

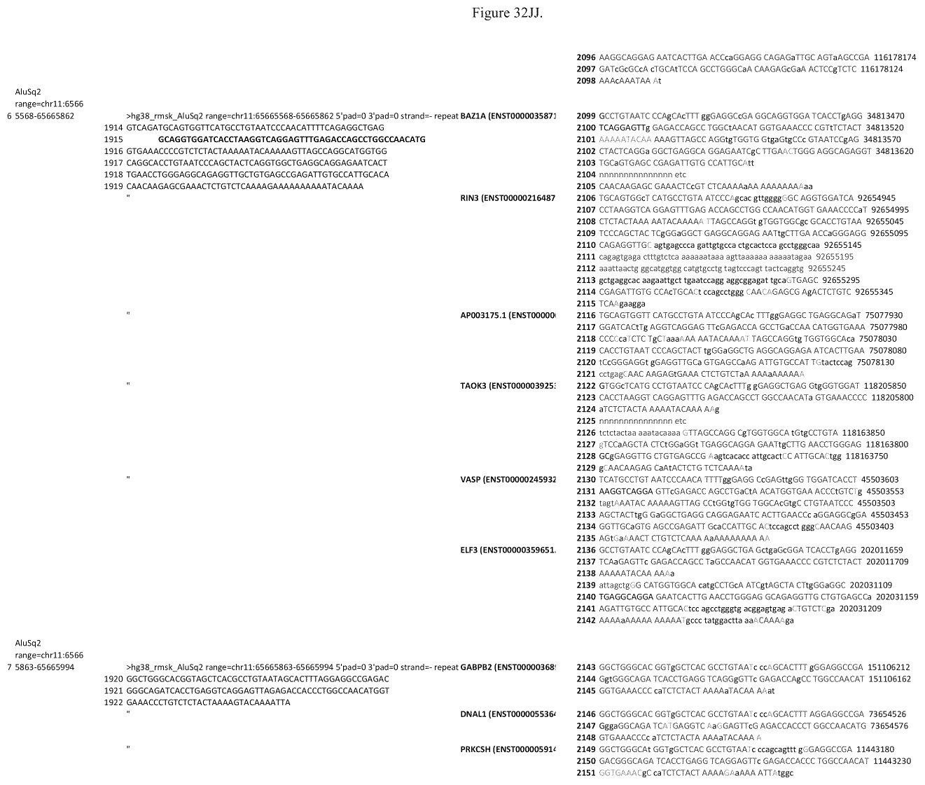

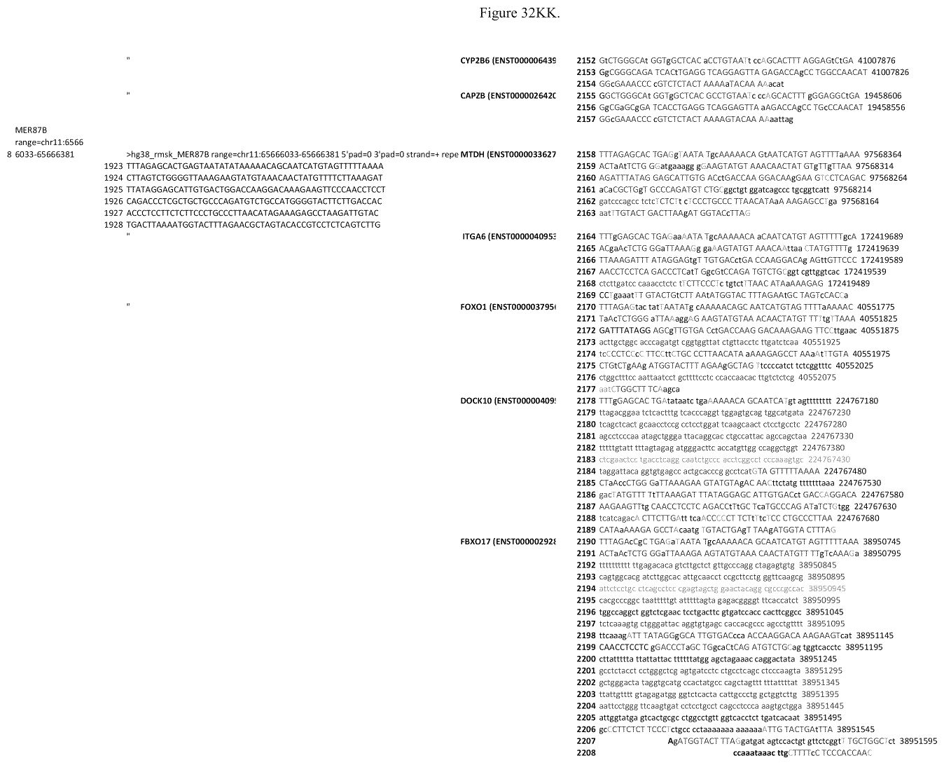

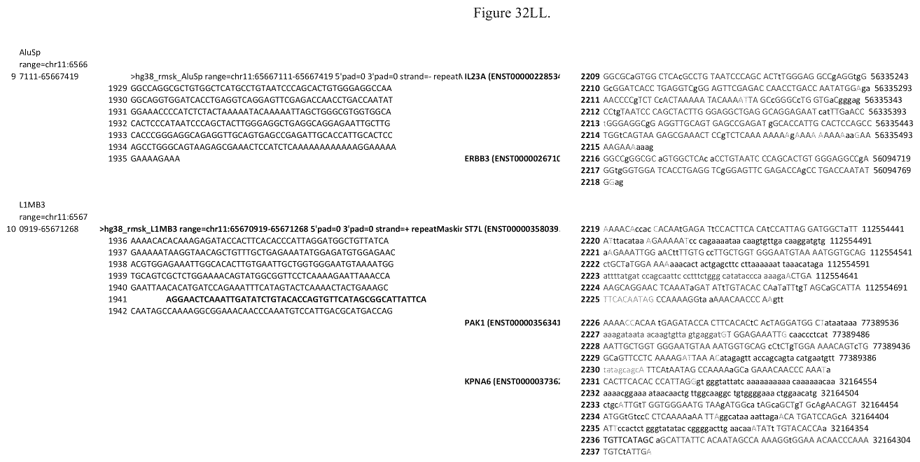

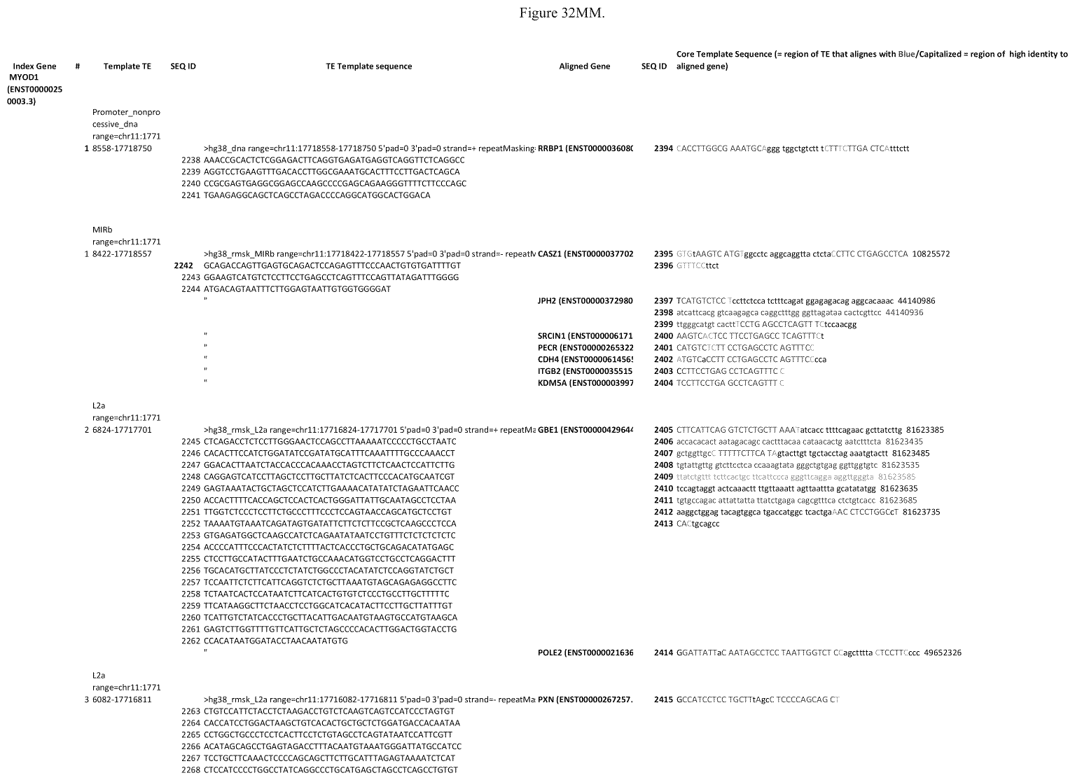

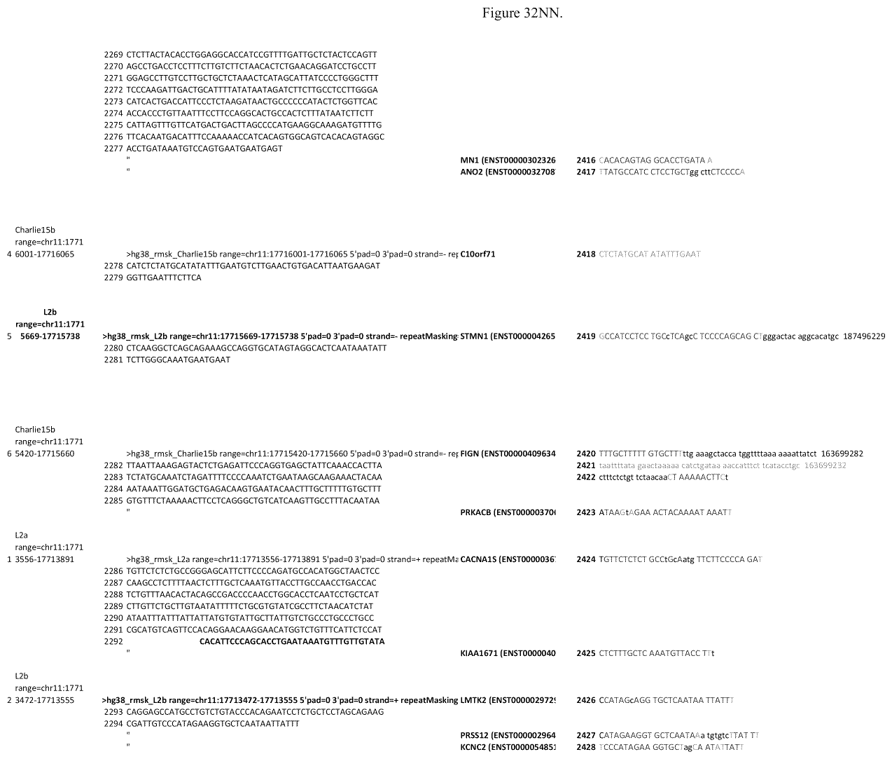

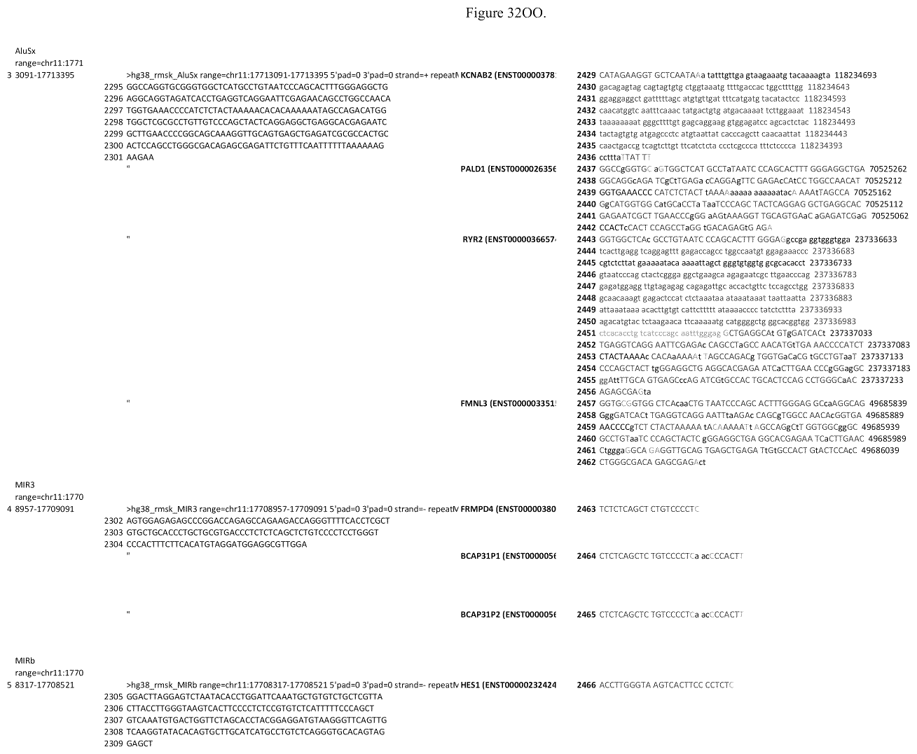

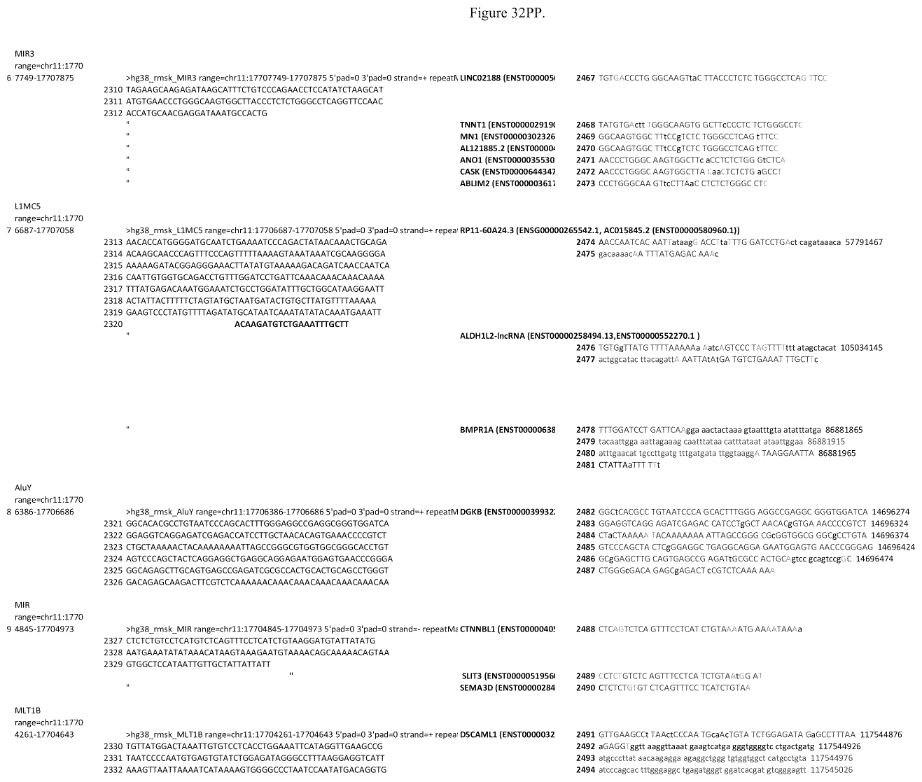

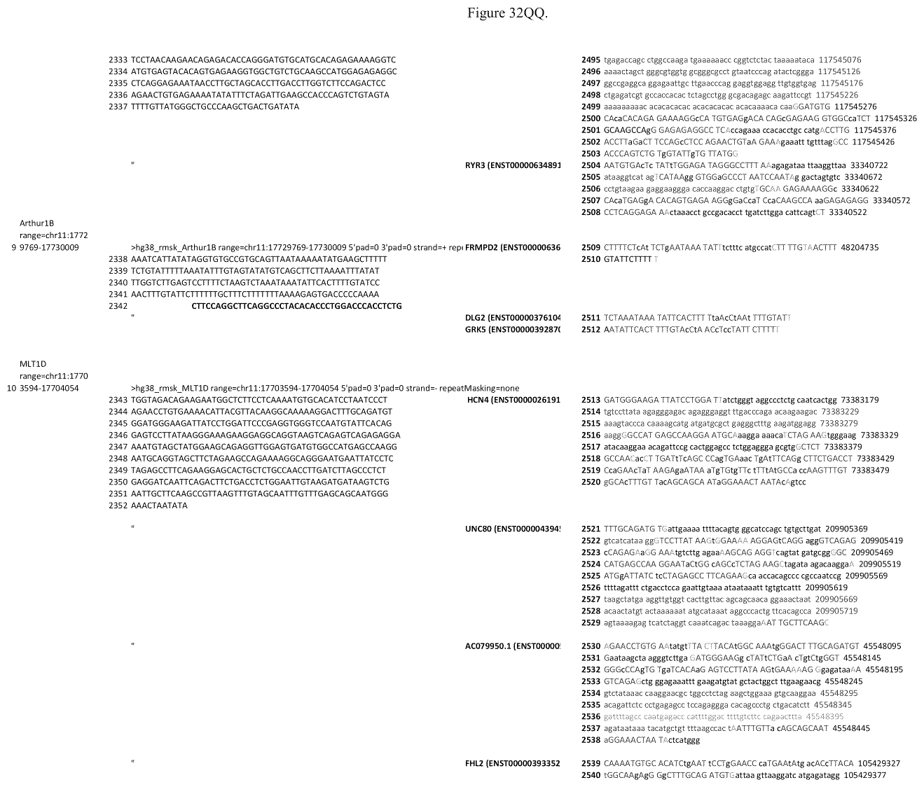

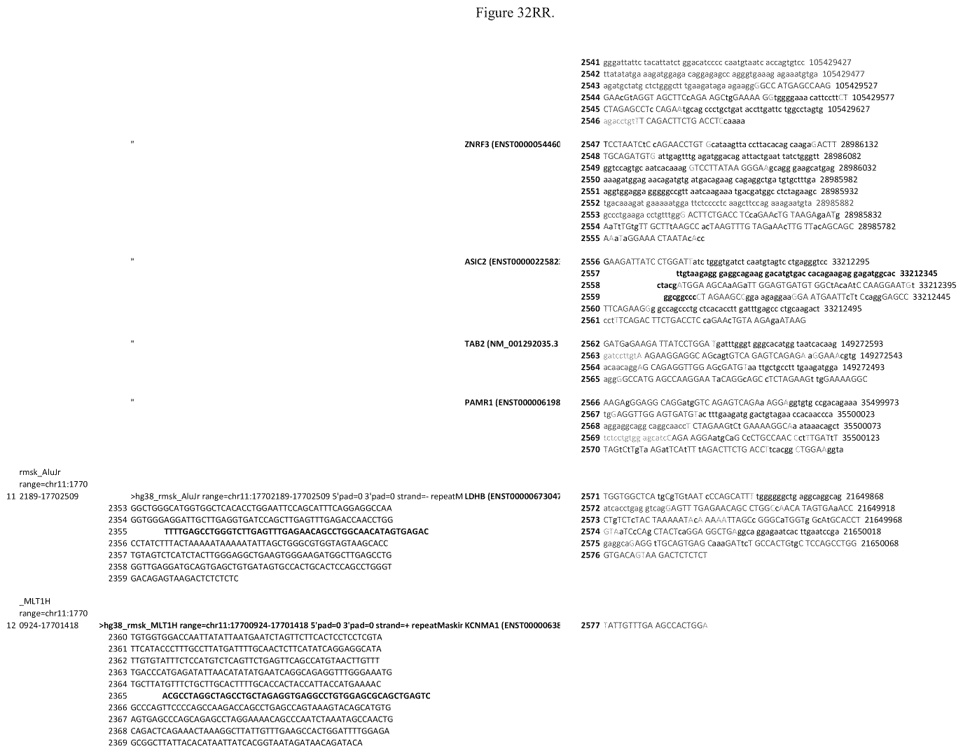

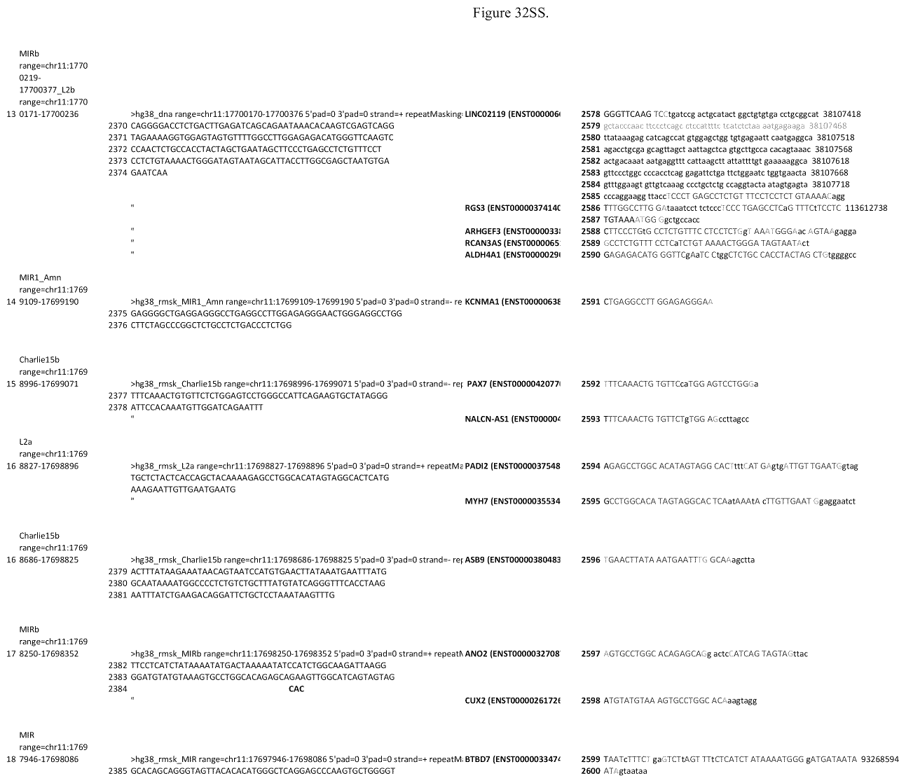

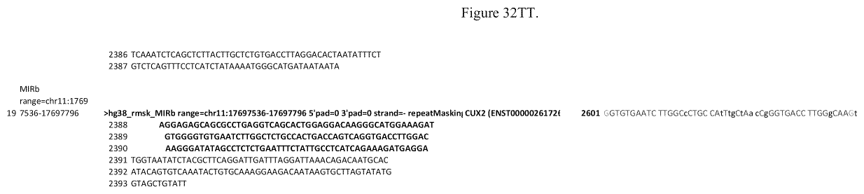

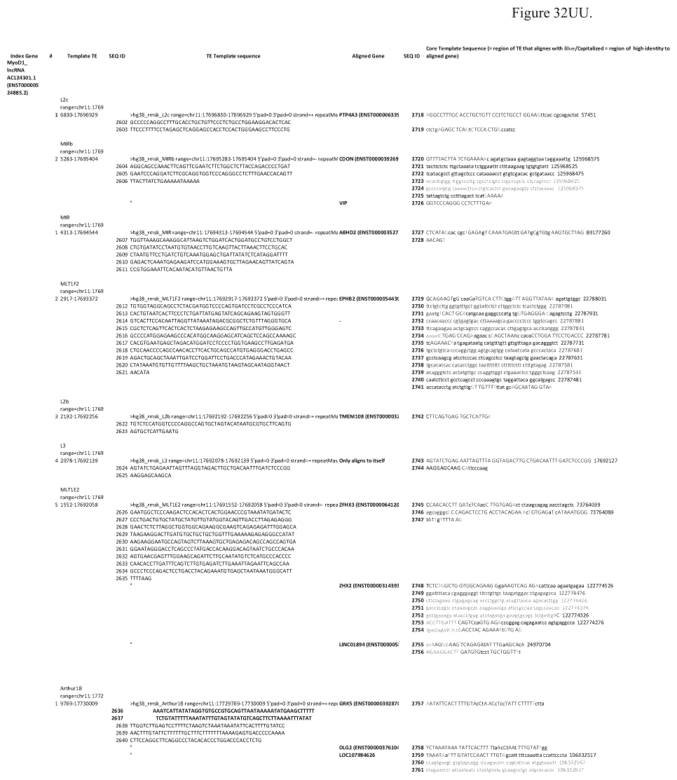

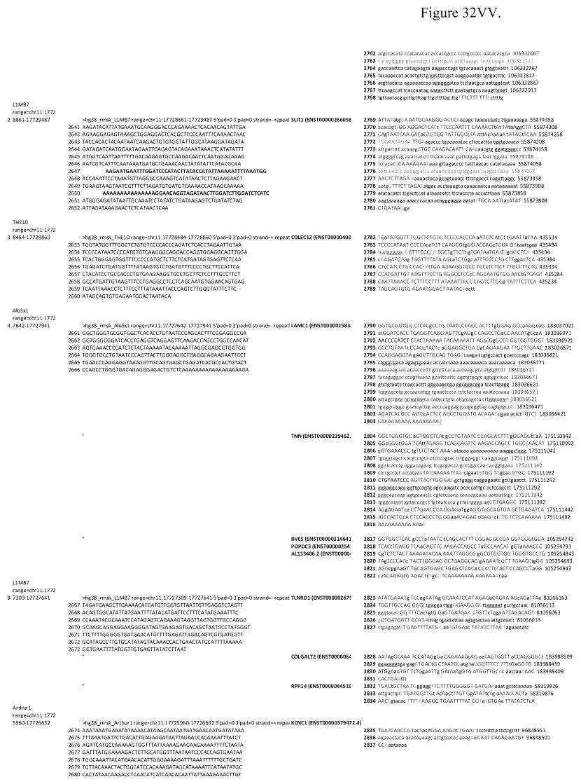

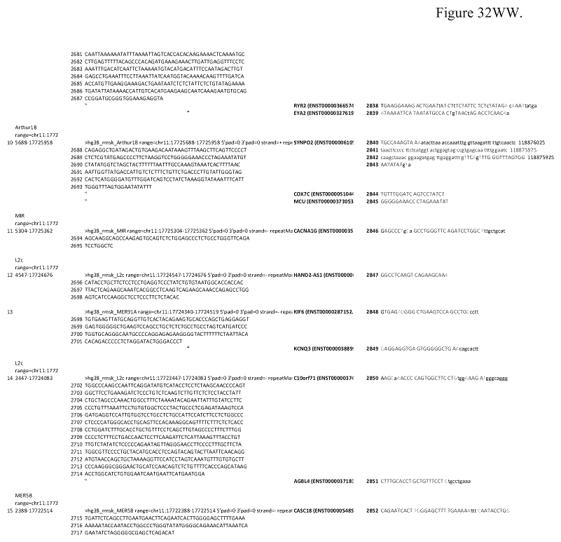

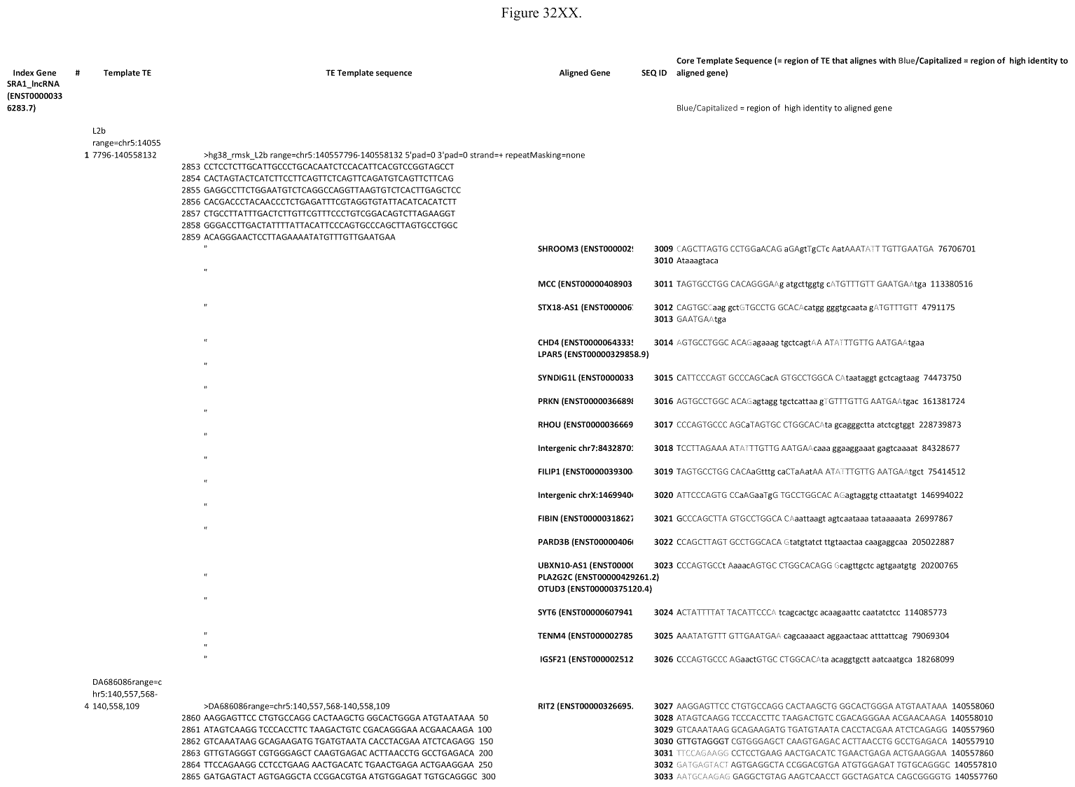

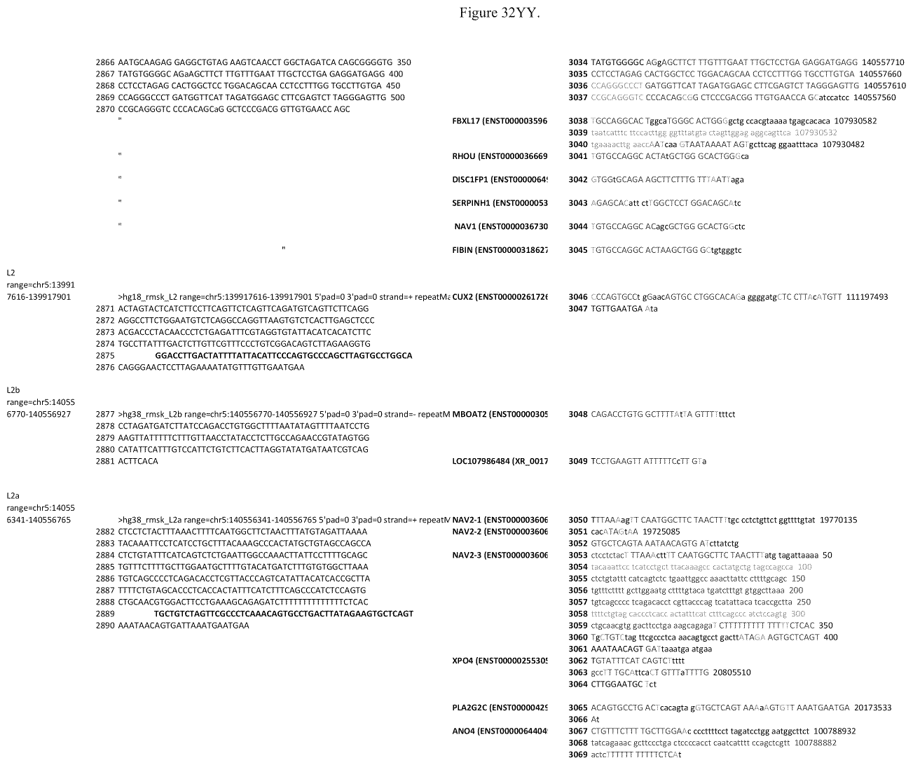

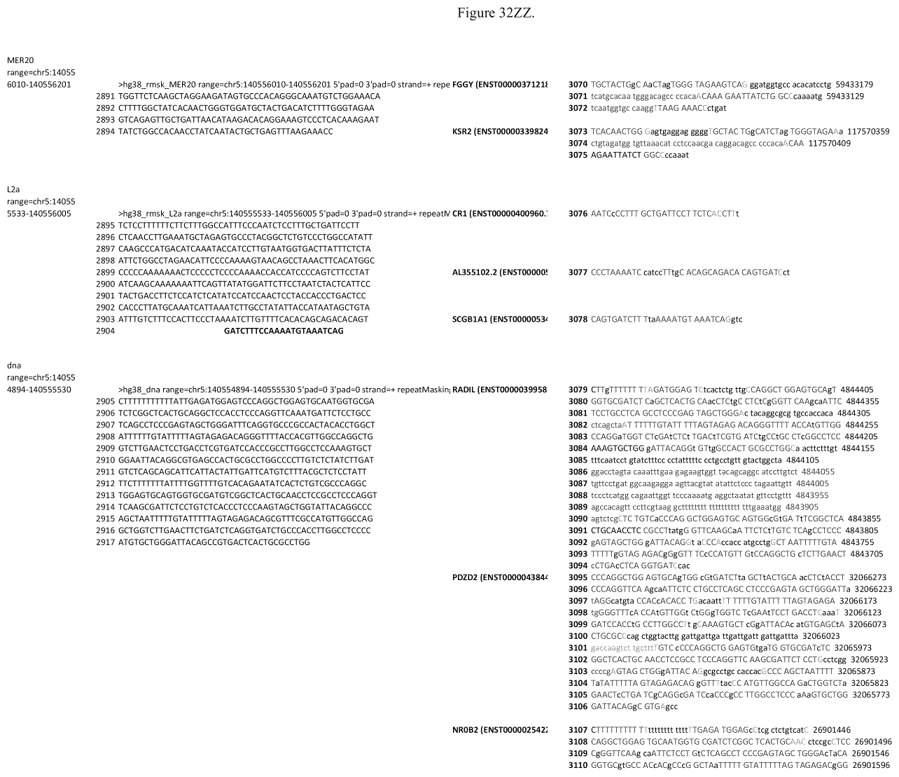

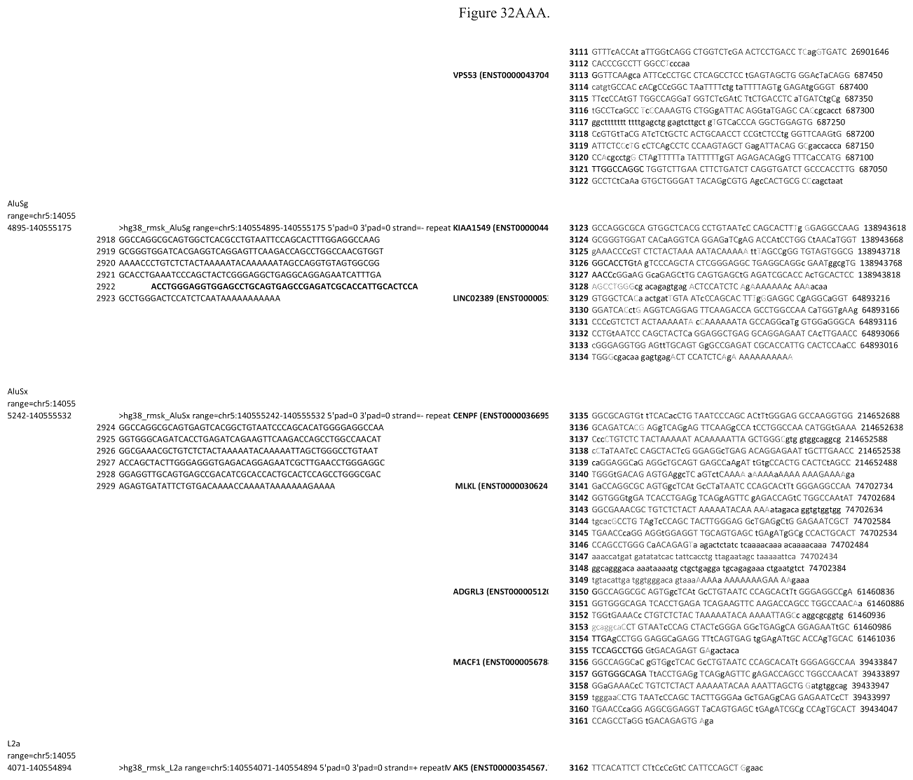

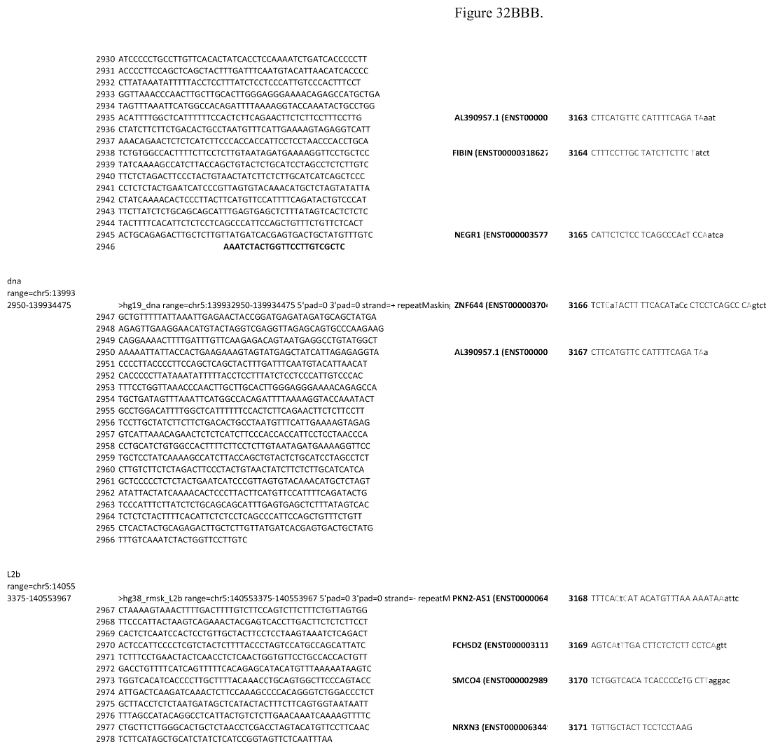

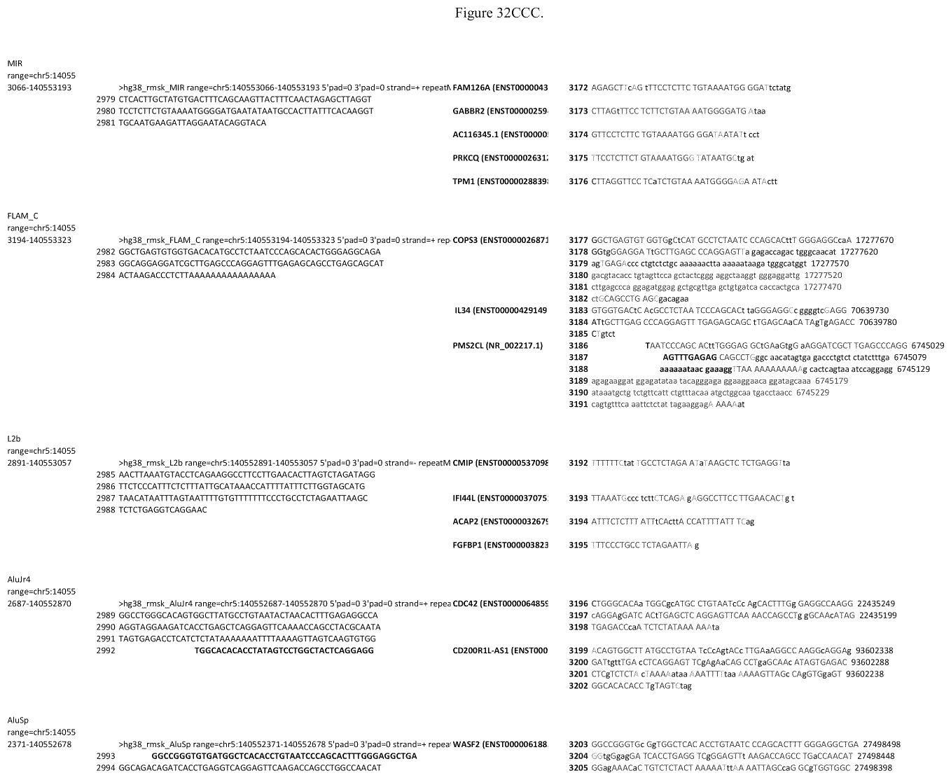

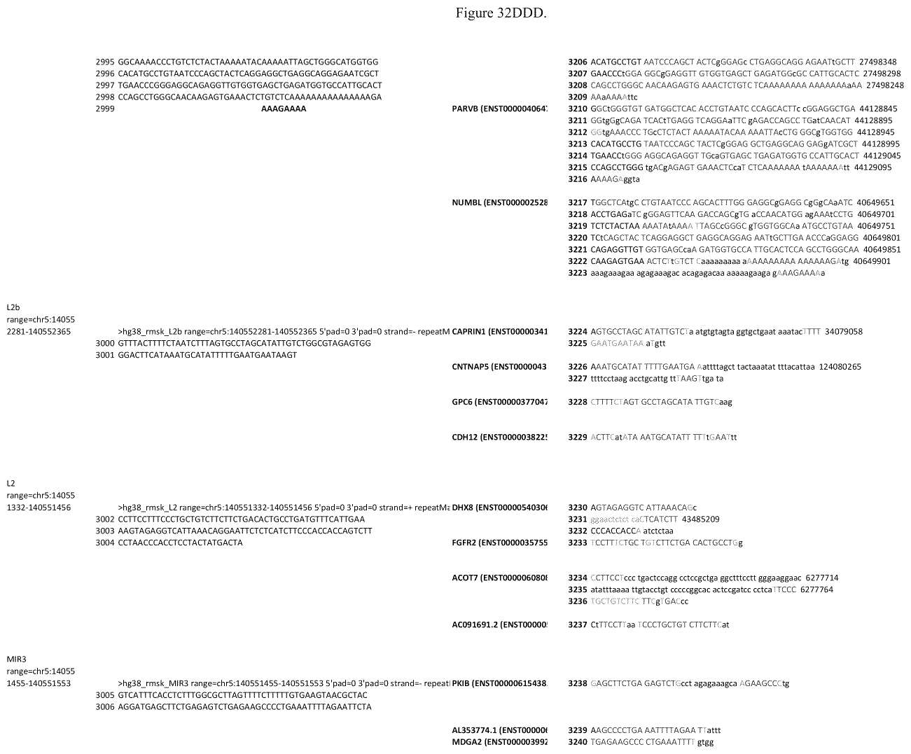

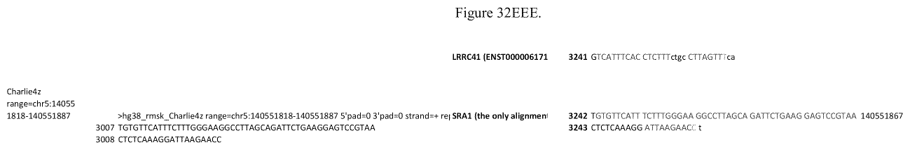

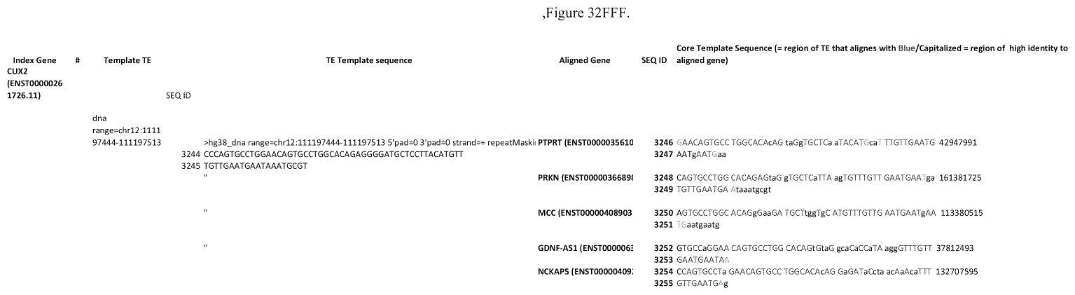

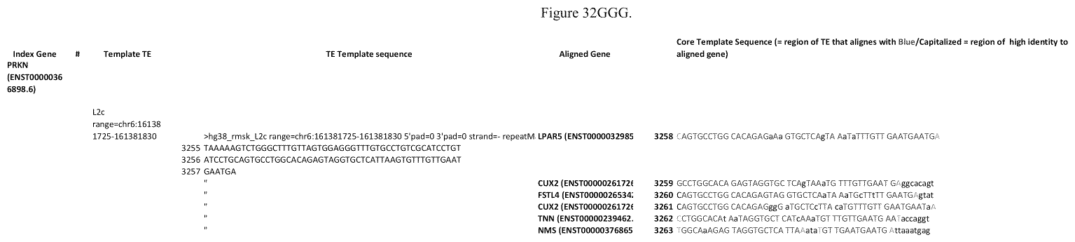

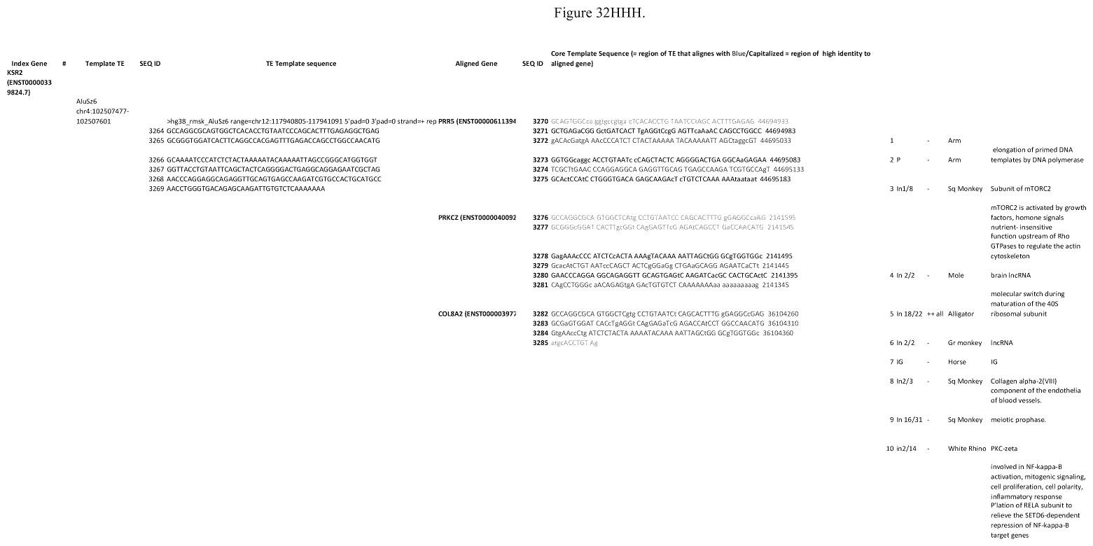

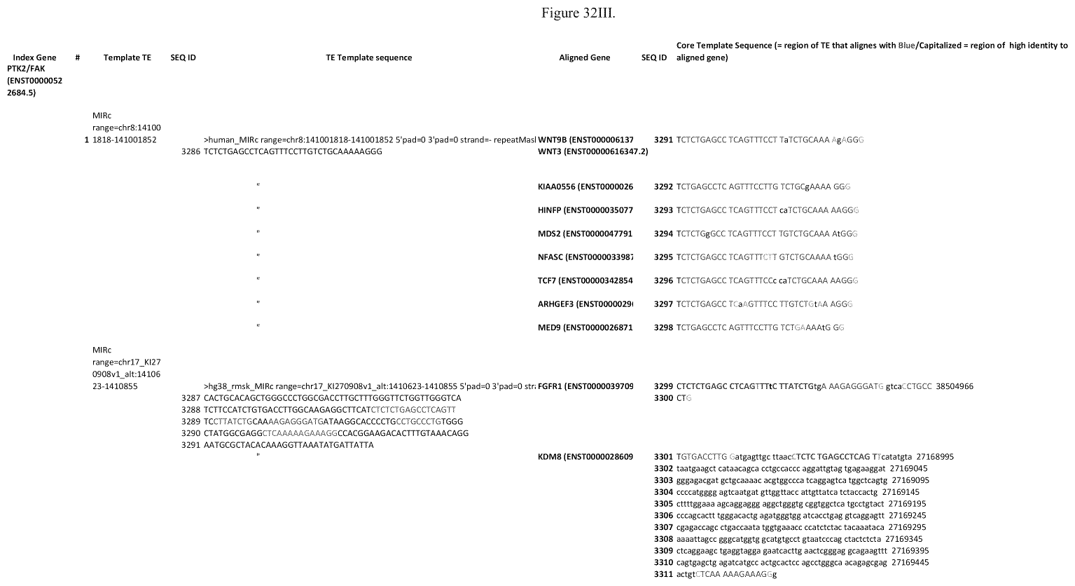

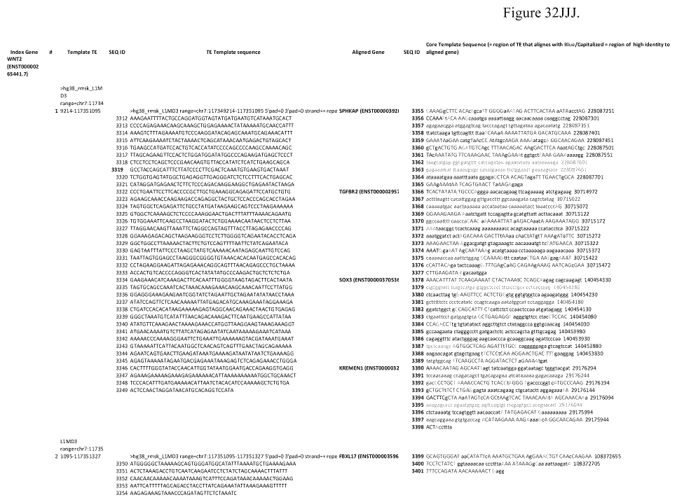

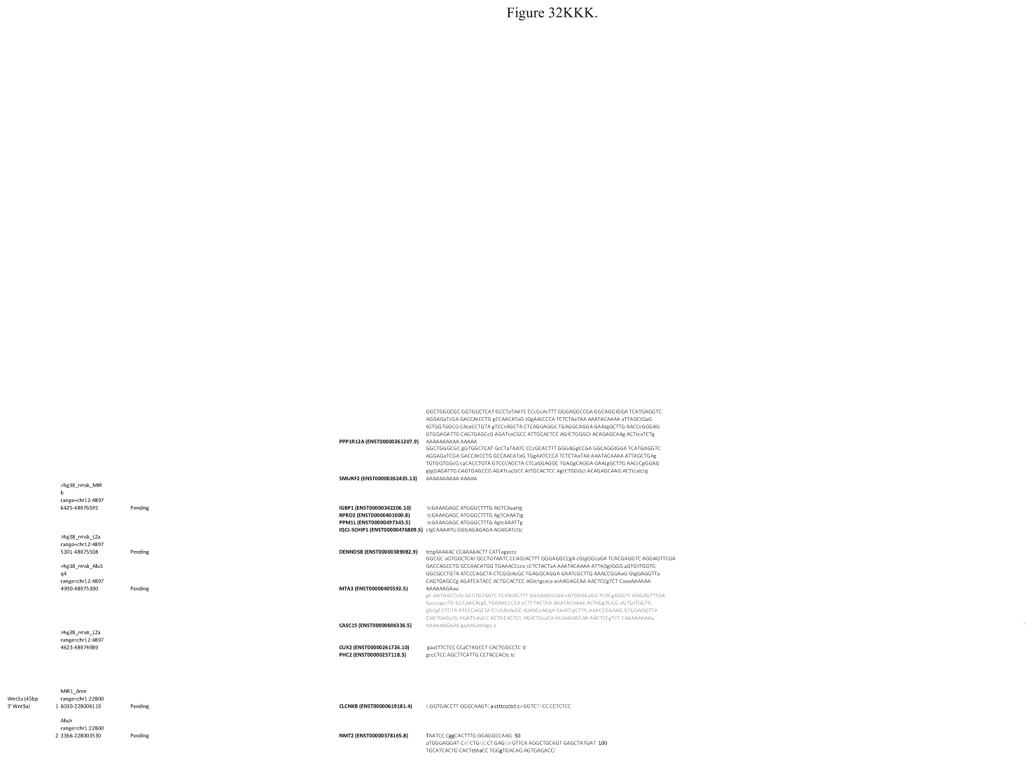

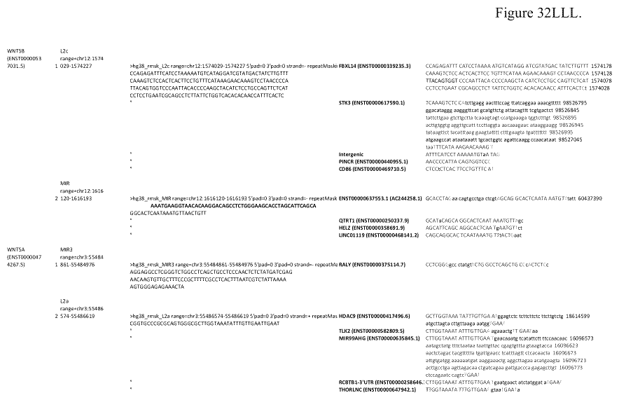

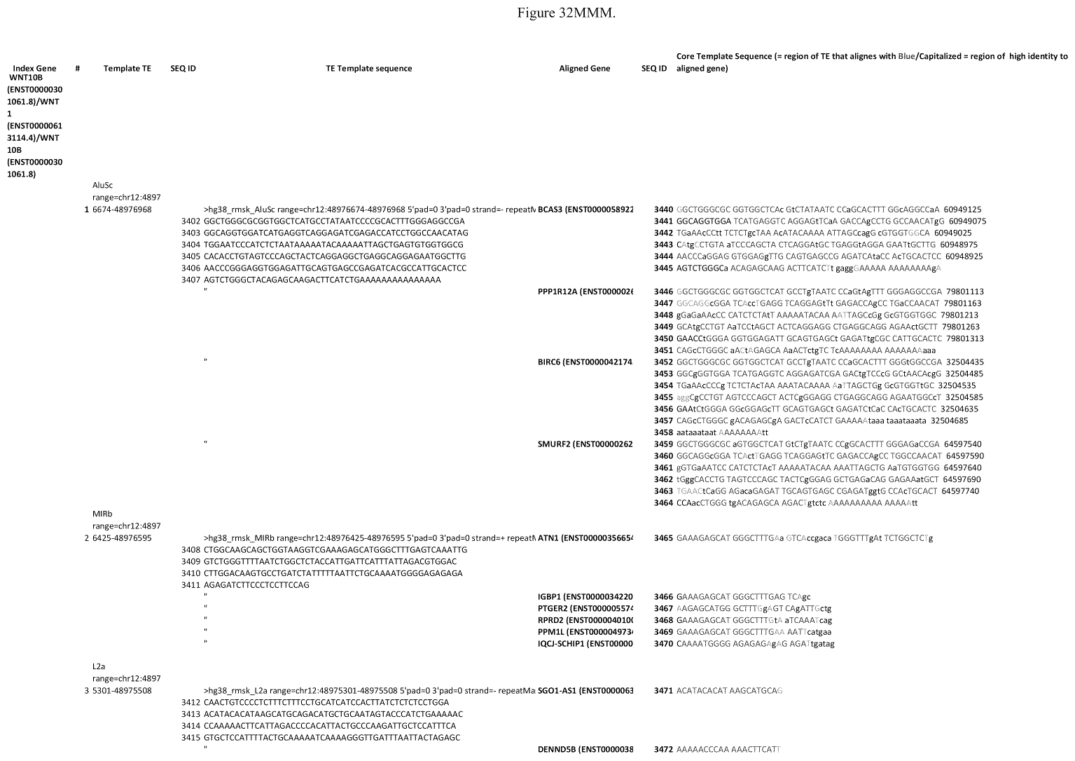

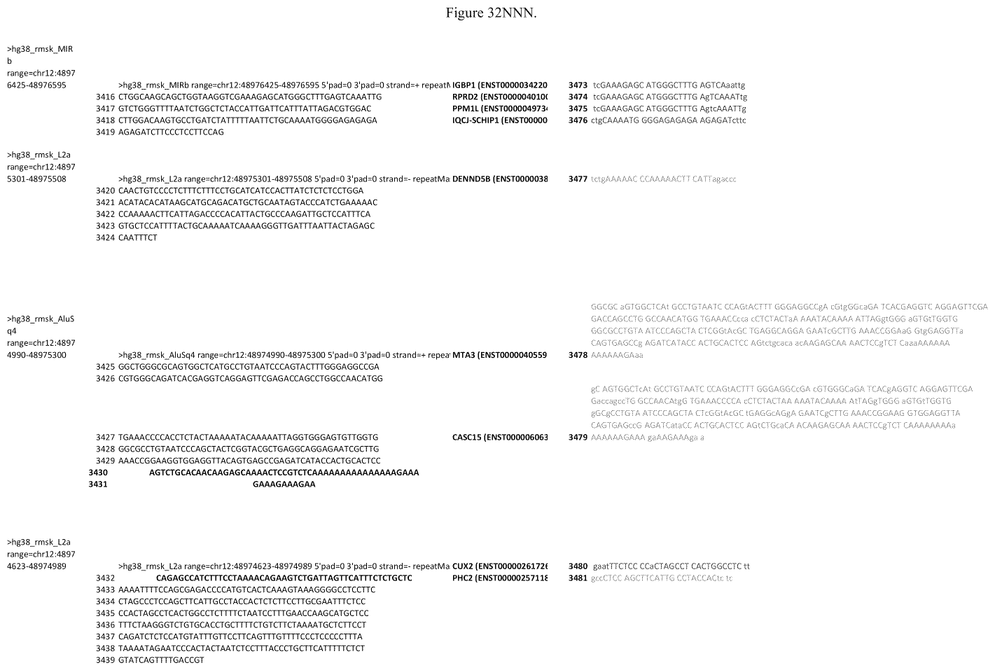

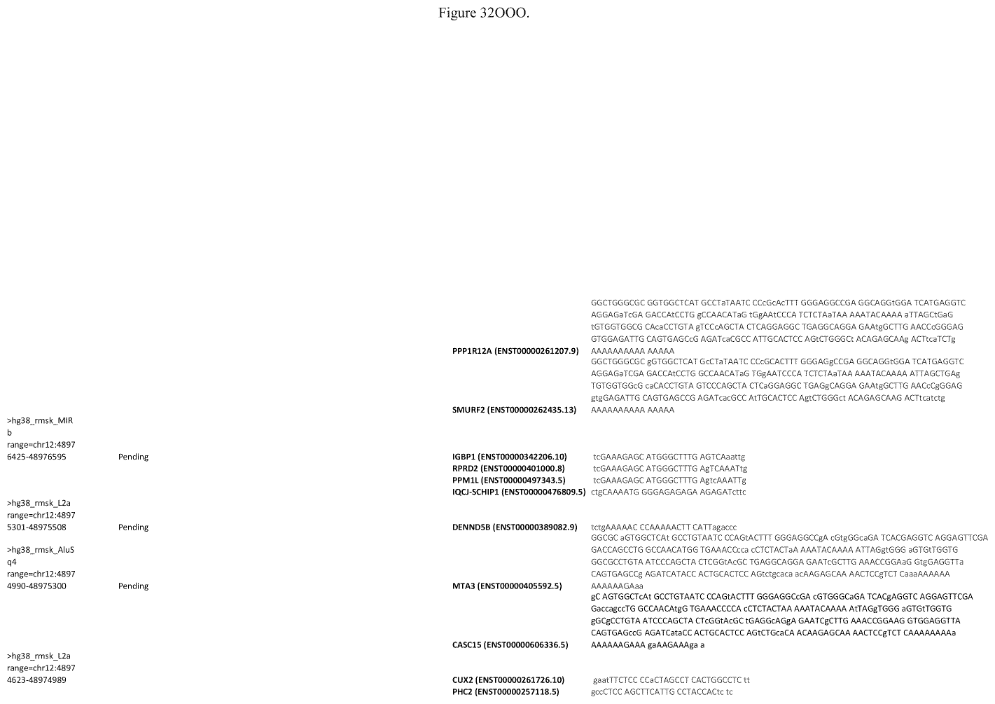

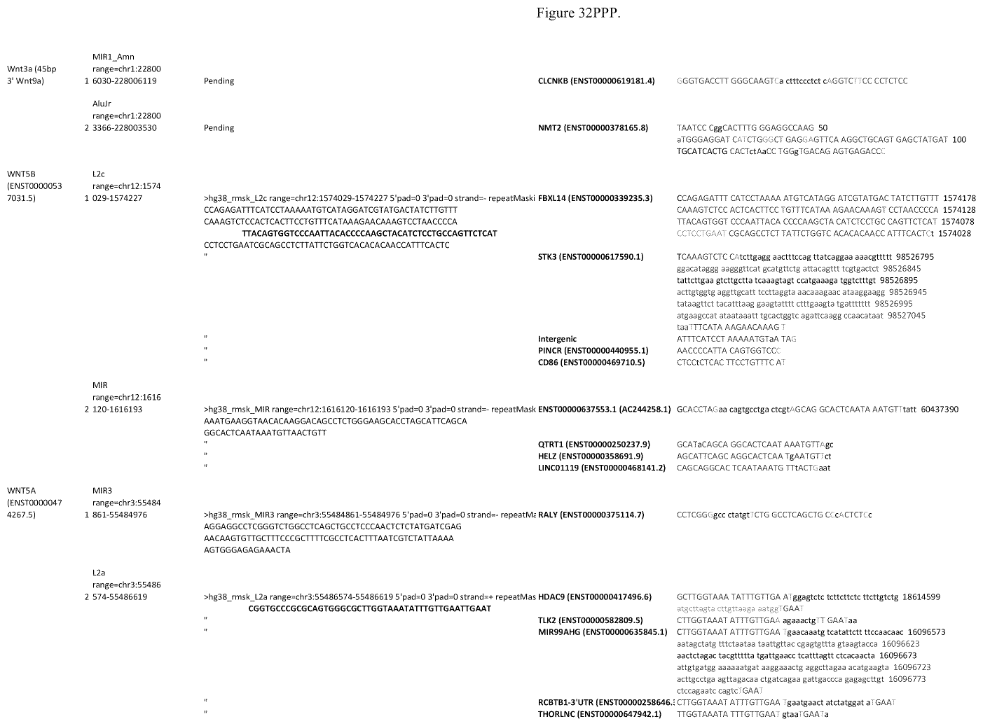

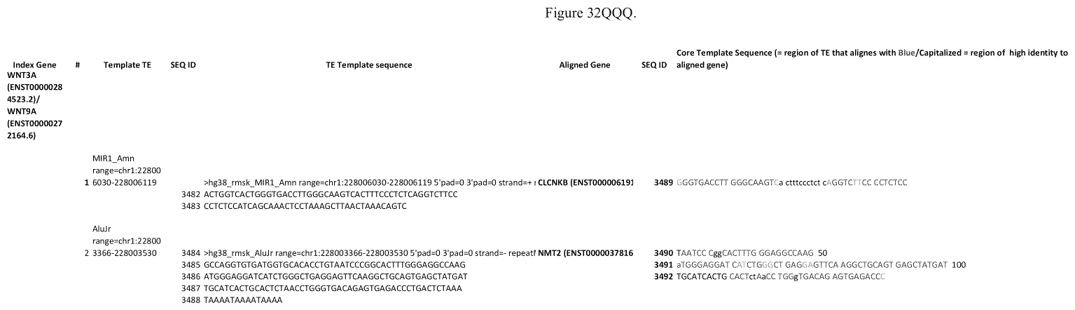

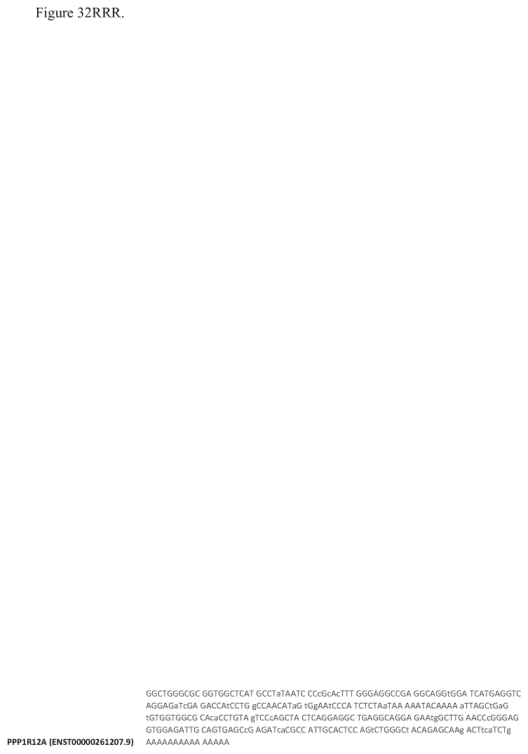

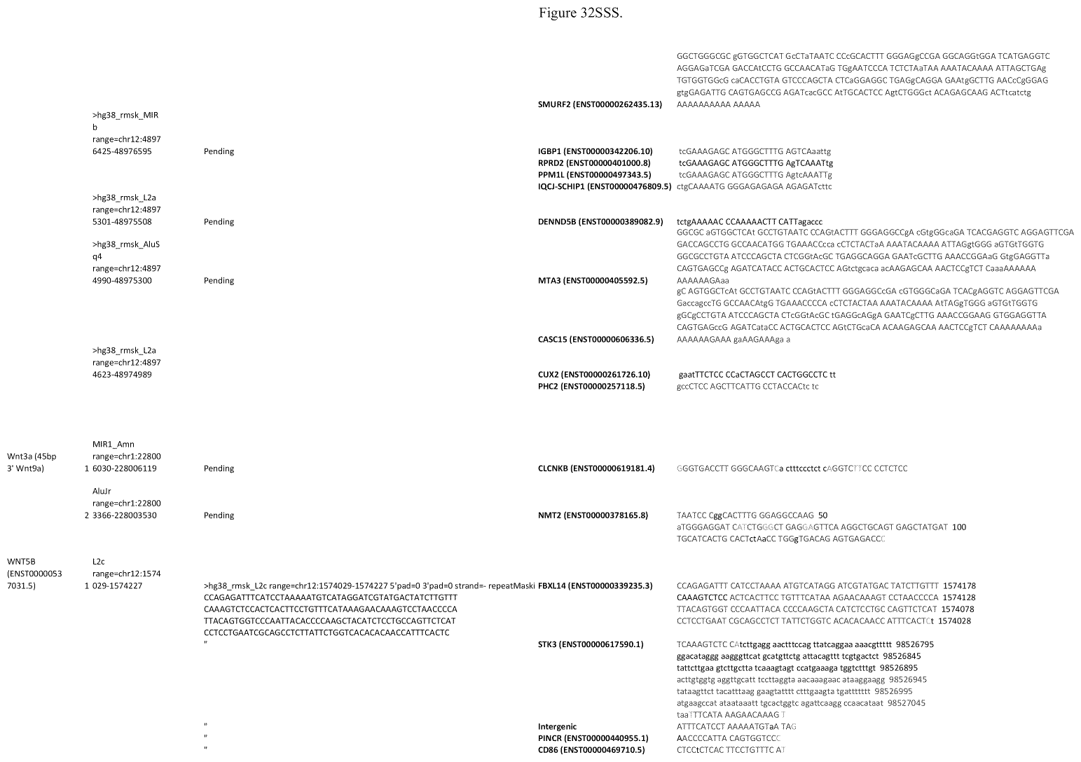

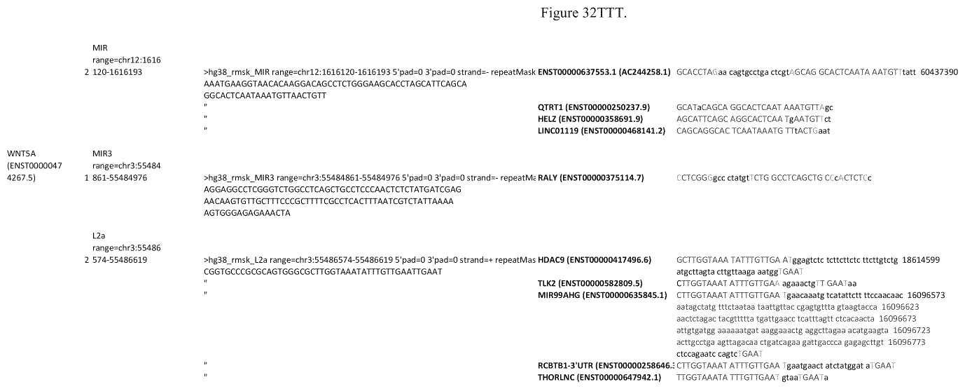

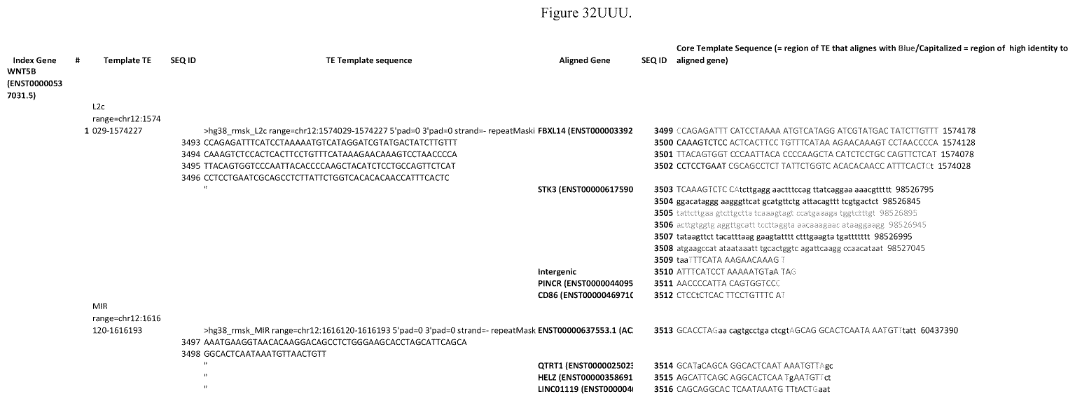

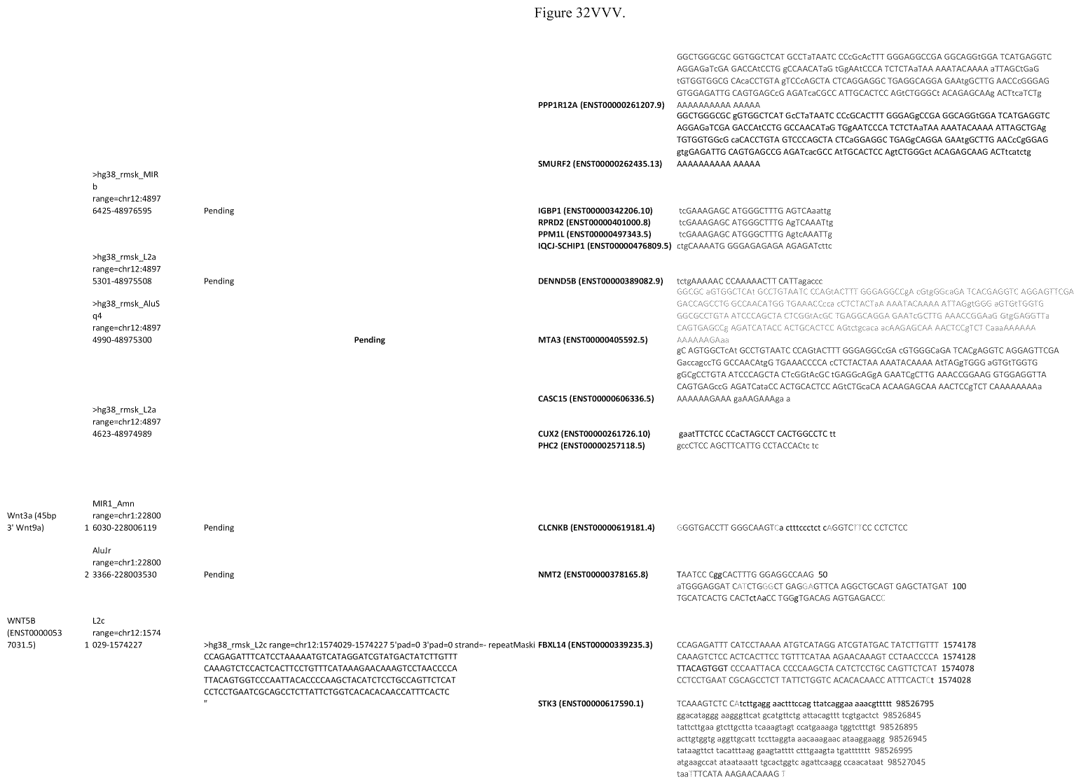

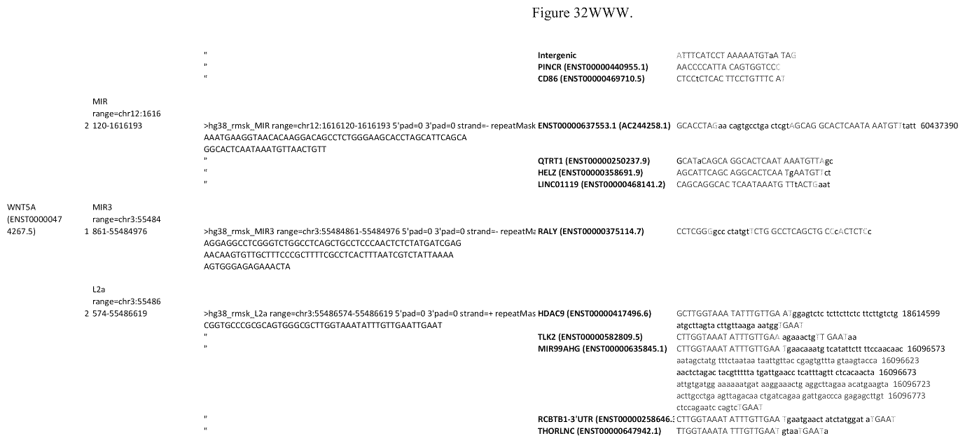

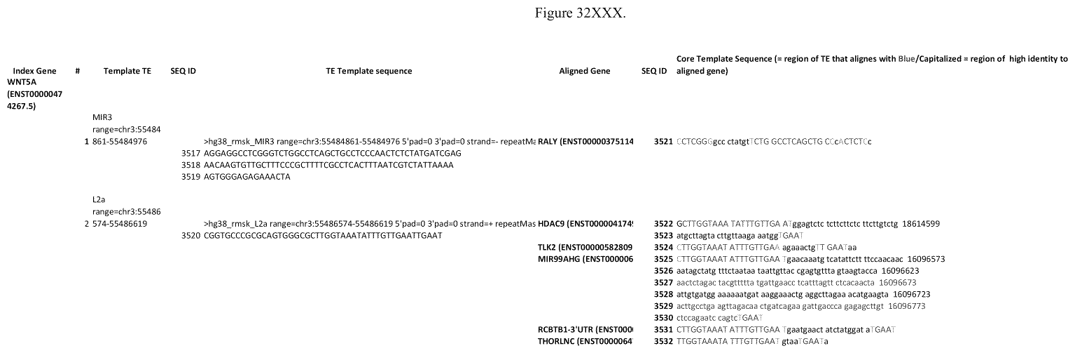

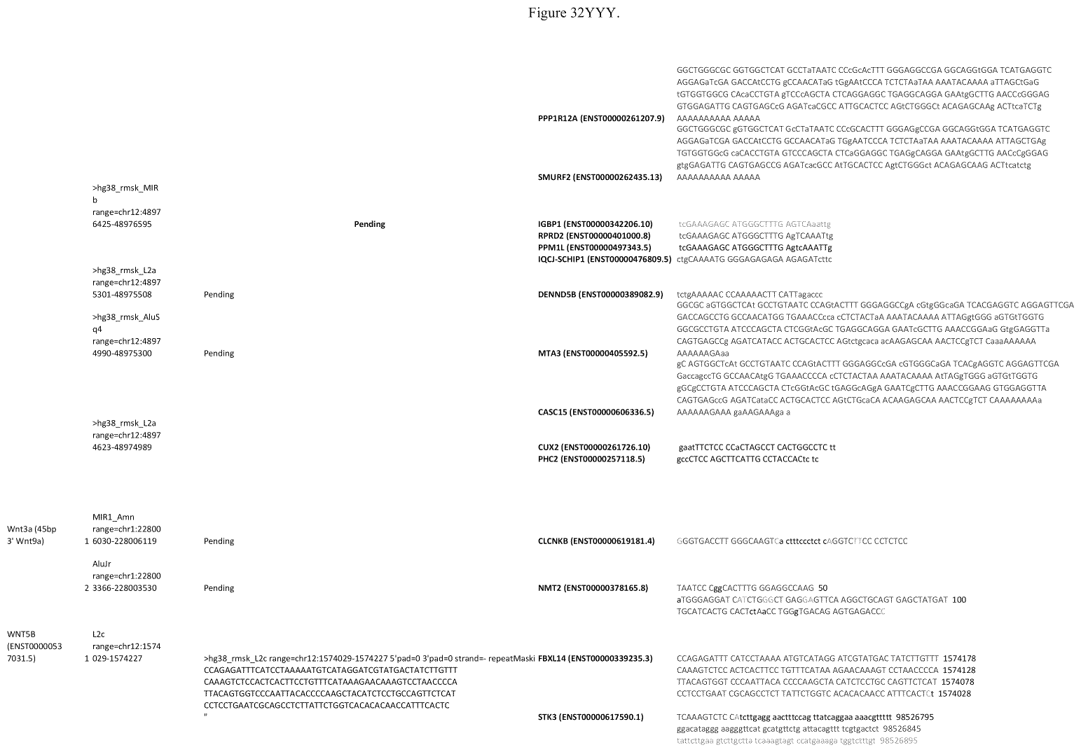

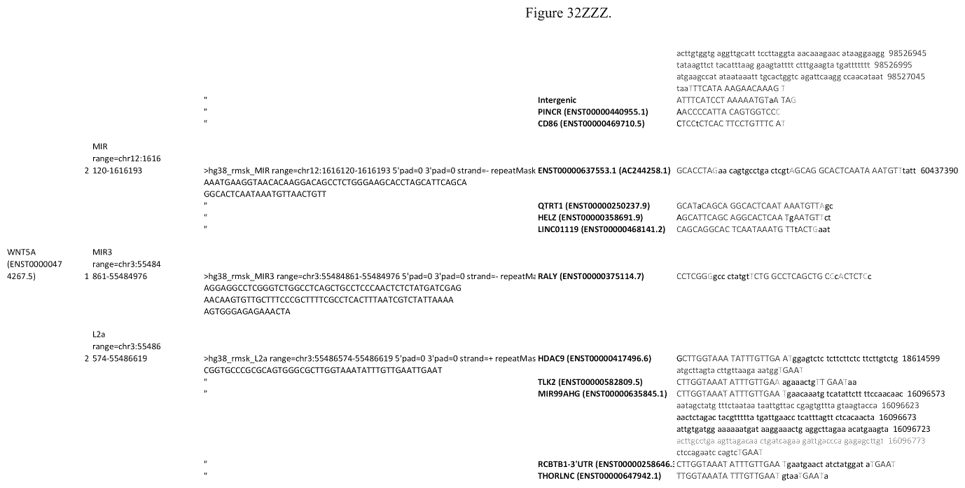

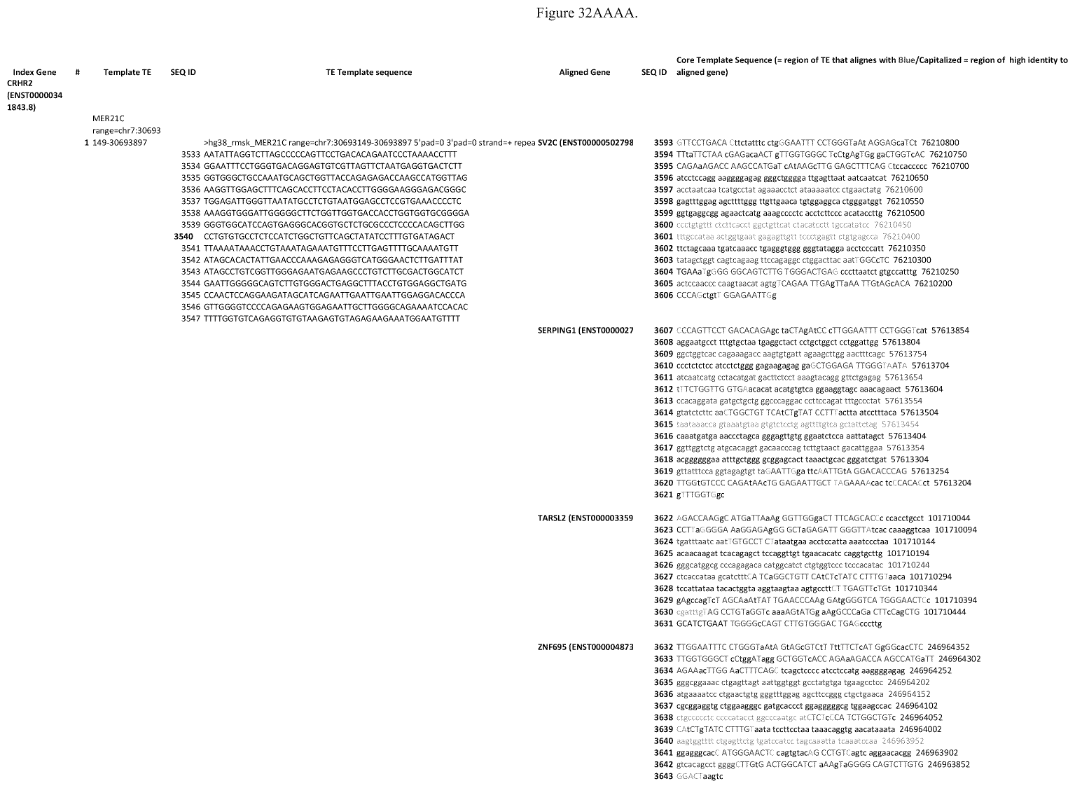

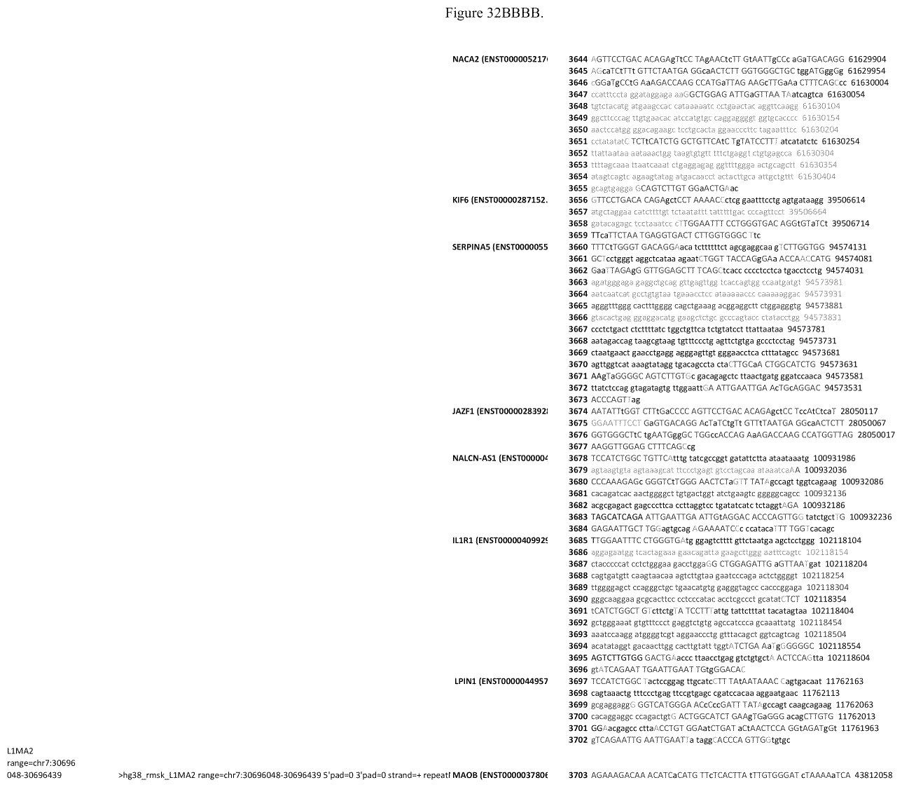

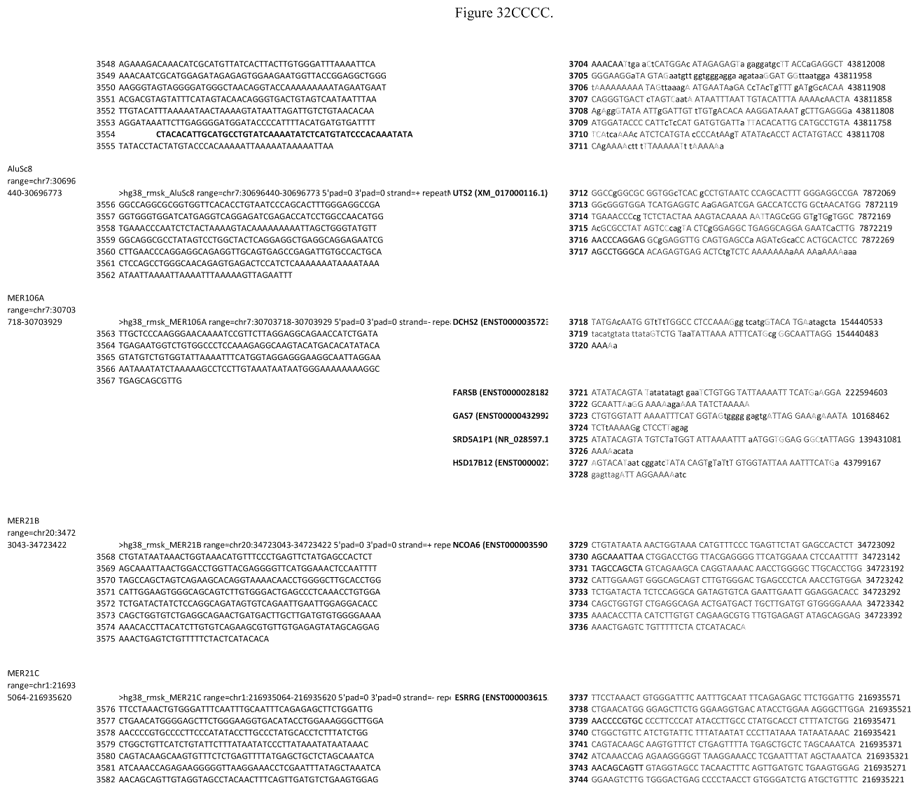

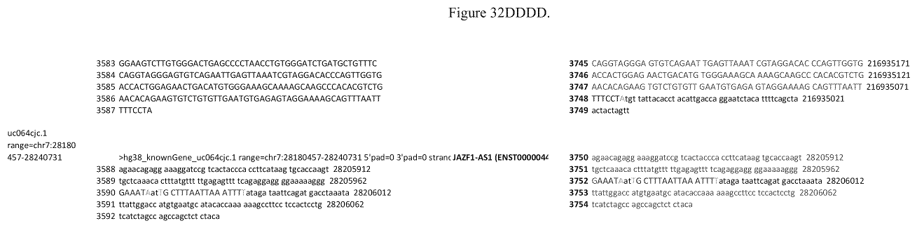

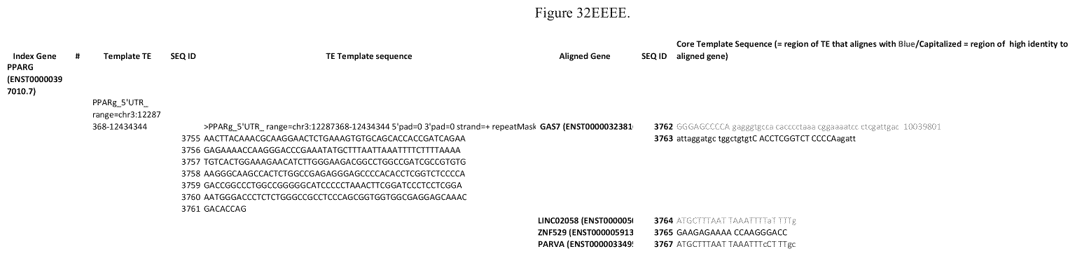

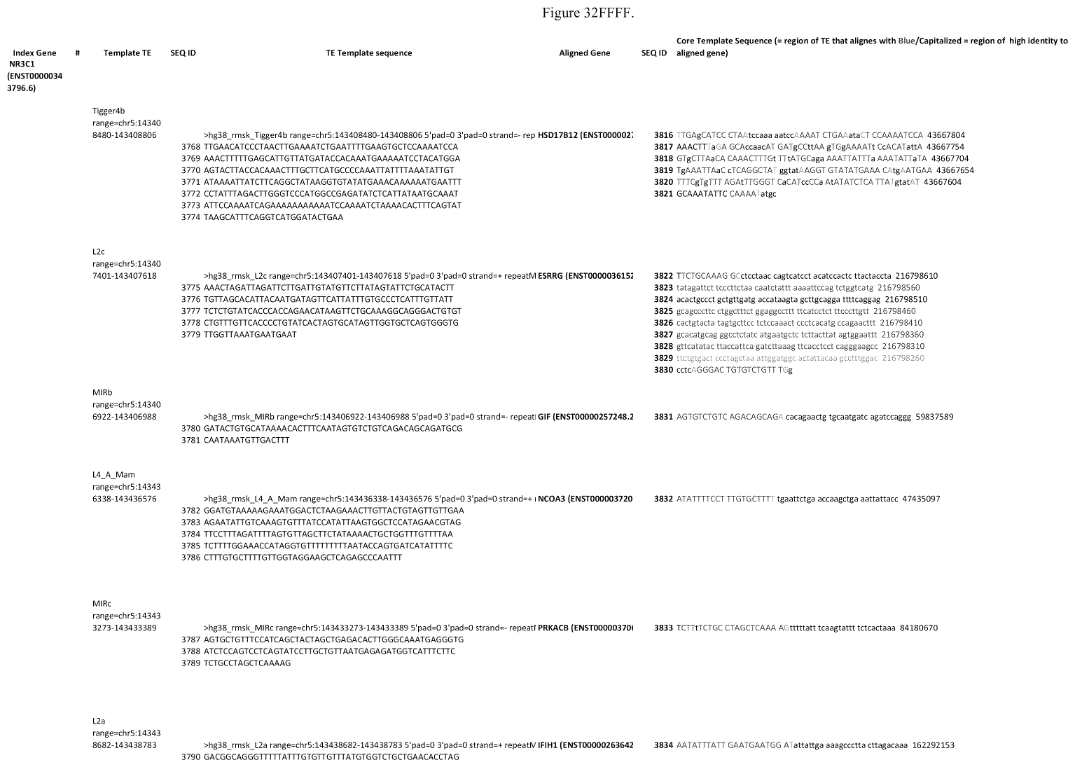

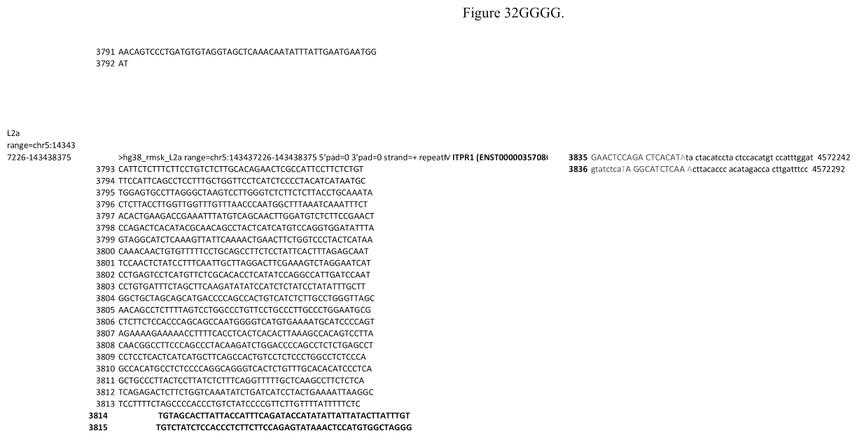

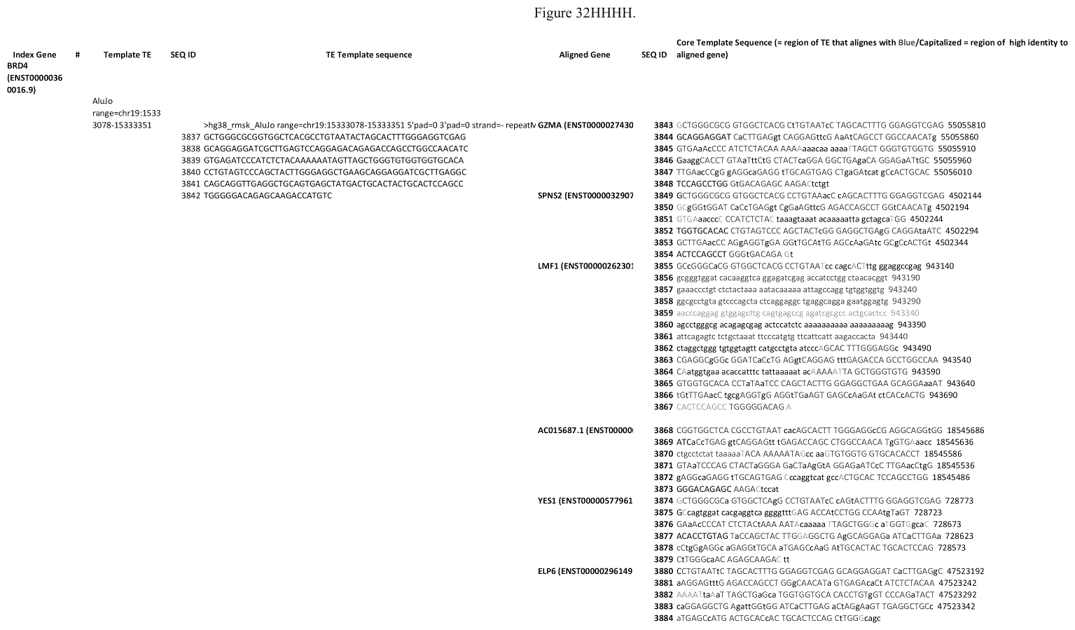

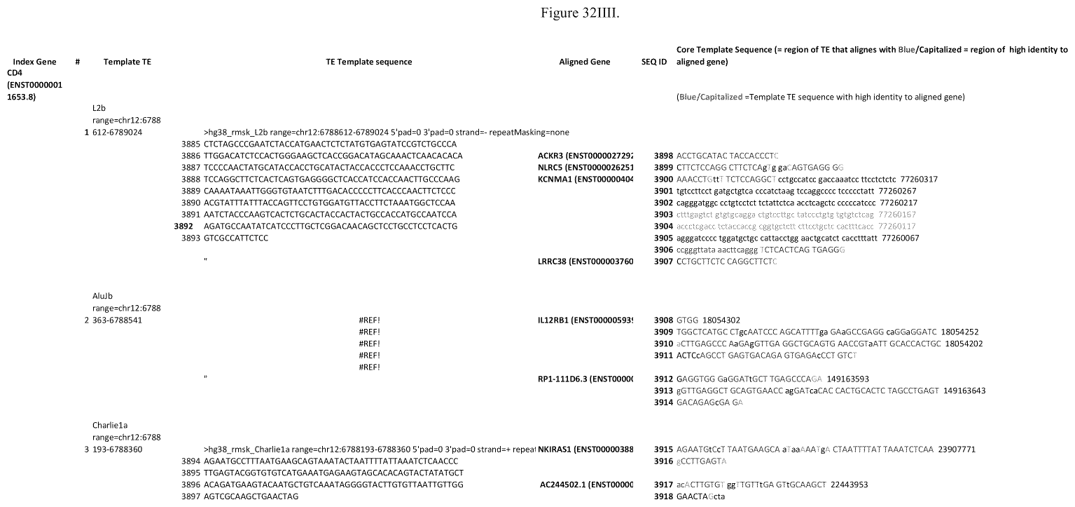

CRHR2 coordinates the endocrine, autonomic and behavioral responses to stress and immune challenge. The in silico method indicated that CRHR2 intron 1 MER21C aligns a gene network that participates in endocrine-mediated lipid metabolism and adipogenesis. The protein: protein interactions within this pathway is confirmed by the STRING database (https://string-db.org) (FIG. 30).

### Example 11: T-Cell Surface Glycoprotein CD4 TEr and Genes of Immune Cells and HIV Binding

T-Cell Surface Glycoprotein CD4, a coreceptor with the T-cell receptor on T lymphocytes, recognizes antigens displayed by antigen presenting cells in the context of class II MHC molecules. It is expressed not only in T lymphocytes, but also in B cells, macrophages, granulocytes, as well as in various regions of the brain, to initiate or augment the early phase of T-cell activation. It is the primary receptor for human immunodeficiency virus-1 (HIV-1). The in silico method indicated that the L2 TEr adjacent to the CD4 promoter transcription start site aligned with high identity to ACKR3, a coreceptor of HIV and NLRC5, a regulator of NFkB and Type 1 Interferon signaling (important for host defense against viruses; Table 12). Interestingly, it also aligned KCNMA1 (potassium channel with role in controlling cell excitability in innate immunity) and a subunit of KCNMA1: LRC38 (potassium channel associated with lymph node carcinoma) (Table 12).

TABLE 12
CD4 transcription start site proximal L2b top 10 alignments

| Hit# | Location | Conser | Expression | Summary | Name | Description |
|---|---|---|---|---|---|---|
| 1 | Pro | Arm | +/−hESC | match to self | CD4 | |
| | | | | | (ENST00000011653.8) | |
| 2 | In 2/29 | Arm | — | voltage and | KCNMA1 | |
| | | | | ca++sensitive | (ENST00000404771.7) | |
| | | | | potassium | | |
| | | | | channels: | | |
| | | | | smooth | | |
| | | | | muscle, | | |
| | | | | neuronal | | |
| | | | | excitability | | |
| | | | | | | Description: The sequence shown here is derived from an |
| | | | | | | Ensembl automatic analysis pipeline and should be |
| | | | | | | considered as preliminary data. (from UniProt Q5SVJ8) |
| | | | | | | RefSeq Summary (NM_001014797): MaxiK channels are |
| | | | | | | large conductance, voltage and calcium-sensitive potassium |
| | | | | | | channels which are fundamental to the control of smooth |
| | | | | | | muscle tone and neuronal excitability. MaxiK channels can |
| | | | | | | be formed by 2 subunits: the pore-forming alpha subunit, |
| | | | | | | which is the product of this gene, and the modulatory beta |
| | | | | | | subunit. Intracellular calcium regulates the physical |
| | | | | | | association between the alpha and beta subunit |
| 3 | In/Ex | Plat | 3+GM78 | regulator of | NLRC5 | |
| | (48/49) | 200mya | | the NF- | (ENST00000262510.10) | |
| | | | | kappa-B and | | |
| | | | | type I | | |
| | | | | interferon | | |
| | | | | signaling | | |
| | | | 3+HELA | homeostatic | | Description: Probable regulator of the NF-kappa-B and type I |
| | | | | control of | | interferon signaling pathways. May also regulate the type II |
| | | | | innate | | interferon signaling pathway. Plays a role in homeostatic |
| | | | | immunity | | control of innate immunity and in antiviral defense |
| | | | | and in | | mechanisms. (from UniProt Q86WI3) |
| | | | | antiviral | | |
| | | | | defense | | |
| | | | | mechanisms | | |
| | | | 2+NHLF | inhibition | | |
| | | | | NFKB | | |
| | | | | activation, | | |
| | | | | negative | | |
| | | | | regulation of | | |
| | | | | type I | | |
| | | | | interferon | | |
| | | | | signaling | | |
| | | | 2+HUVEC | | | |
| | | | 2+HSMM | | | |
| | | | 2+NHEK | | | |
| | | | −HepG2 | | | |
| | | | −K562 | | | |
| | | | −ESC | | | |
| 4 | In 1/27 | Arm | +/−GM78 | B-TFIID | BTAF1 | |
| | | | | TATA-box | (ENST00000265990.10) | |
| | | | | binding | D | |
| | | | | drives the | | Description: Homo sapiens B-TFIID TATA-box binding |
| | | | | dissociation | | protein associated factor 1 (BTAF1), mRNA. (from RefSeq |
| | | | | of TBP from | | NM_003972) |
| | | | | DNA | | |
| | | | | | | RefSeq Summary (NM_003972): This gene encodes a TAF |
| | | | | | | (TATA box-binding protein-associated factor), which |
| | | | | | | associates with TBP (TATA box-binding protein) to form the |
| | | | | | | B-TFIID complex that is required for transcription initiation |
| | | | | | | of genes by RNA polymerase II. This TAF has DNA- |
| | | | | | | dependent ATPase activity, which drives the |
| | | | | | | dissociation of TBP from DNA, freeing the TBP to |
| | | | | | | associate with other TATA boxes or TATA-less |
| | | | | | | promoters. [provided by RefSeq, September 2011] |
| 5 | IG | HedgHog | +/−HSMM | | IG | |
| 6 | IG | Aard | — | | IG | |
| 7 | In 13/17 | Arm/ | — | organizes the | SDCCAG8 | |
| | | TasDev | | centrosome | (ENST00000366541.7) | |
| | | | | during | | |
| | | | | interphase | | |
| | | | | and mitosis. | | |
| | | | | | | Description: Homo sapiens serologically defined colon |
| | | | | | | cancer antigen 8 (SDCCAG8), mRNA. (from RefSeq |
| | | | | | | NM_006642) |
| | | | | | | RefSeq Summary (NM_006642): This gene encodes a |
| | | | | | | centrosome associated protein. This protein may be involved |
| | | | | | | in organizing the centrosome during interphase and mitosis. |
| | | | | | | Mutations in this gene are associated with retinal-renal |
| | | | | | | ciliopathy. |
| 8 | Pro | X. Trop | — | collagen type | COL20A1 | |
| | | | | XX | (ENST00000358894.10) | |
| | | | | | | Description: Homo sapiens collagen type XX alpha 1 |
| | | | | | | (COL20A1), mRNA. (from RefSeq NM_020882) |
| | | | | | | a Protein Coding gene. Among its related pathways are |
| | | | | | | Phospholipase-C Pathway and Collagen chain trimerization. |
| | | | | | | An important paralog of this gene is COL14A1. |
| | | | | | | SUBCELLULAR LOCATION: Secreted, extracellular space |
| | | | | | | (Probable). |
| | | | | | | TISSUE SPECIFICITY: High expression in heart, lung, liver, |
| | | | | | | skeletal muscle, kidney, pancreas, spleen, testis, ovary, |
| | | | | | | subthalamic nucleus and fetal liver. Weak expression in |
| | | | | | | other tissues tested. |
| 9 | ~45kb 3′ | Arm | — | Along with | ACKR3 | |
| | | | | CD4, | (ENST00000272928.3) | |
| | | | | coreceptorwith | | |
| | | | | CXCR4 for | | |
| | | | | HIV | | |
| | | | | G-protein | | Description: Homo sapiens atypical chemokine receptor 3 |
| | | | | coupled | | (ACKR3), mRNA. (from RefSeq NM_020311) |
| | | | | receptor | | |
| | | | | family, | | |
| | | | | Atypical | | |
| | | | | chemokine | | |
| | | | | receptor (no | | |
| | | | | known | | |
| | | | | ligand) | | |
| | | | | | | RefSeq Summary (NM_020311): This gene encodes a |
| | | | | | | member of the G-protein coupled receptor family. Although |
| | | | | | | this protein was earlier thought to be a receptor for |
| | | | | | | vasoactive intestinal peptide (VIP), it is now considered to be |
| | | | | | | an orphan receptor, in that its endogenous ligand has not |
| | | | | | | been identified. The protein is also a coreceptor for human |
| | | | | | | immunodeficiency viruses (HIV). |
| | | | | | | Atypical chemokine receptor that controls chemokine levels |
| | | | | | | and localization via high-affinity chemokine binding that is |
| | | | | | | uncoupled from classic ligand-driven signal transduction |
| | | | | | | cascades, resulting instead in chemokine sequestration, |
| | | | | | | degradation, or transcytosis. Also known as interceptor |
| | | | | | | (internalizing receptor) or chemokine-scavenging receptor or |
| | | | | | | chemokine decoy receptor. Acts as a receptor for |
| | | | | | | chemokines CXCL11 and CXCL12/SDF1. Chemokine binding |
| | | | | | | does not activate G-protein-mediated signal transduction |
| | | | | | | but instead induces beta-arrestin recruitment, leading to |
| | | | | | | ligand internalization and activation of MAPK signaling |
| | | | | | | pathway. Required for regulation of CXCR4 protein levels in |
| | | | | | | migrating interneurons, thereby adapting their chemokine |
| | | | | | | responsiveness. In glioma cells, transduces signals via |
| | | | | | | MEK/ERK pathway, mediating resistance to apoptosis. |
| | | | | | | Promotes cell growth and survival. Not involved in cell |
| | | | | | | migration, adhesion or proliferation of normal |
| | | | | | | hematopoietic progenitors but activated by CXCL11 in |
| | | | | | | malignant hemapoietic cells, leading to phosphorylation of |
| | | | | | | ERK1/2 (MAPK3/MAPK1) and enhanced cell adhesion and |
| | | | | | | migration. Plays a regulatory role in CXCR4-mediated |
| | | | | | | activation of cell surface integrins by CXCL12. Required for |
| | | | | | | heart valve development. Acts as coreceptor with CXCR4 for |
| | | | | | | a restricted number of HIV isolates. |
| | | | | | | Acts as coreceptor with CXCR4 for a restricted number of |
| | | | | | | HIV isolates. |
| 10 | In 1/1 | Arm | — | Leucine-rich | LRRC38 | |
| | | | | repeat- | (ENST00000376085.4) | |
| | | | | containing | | |
| | | | | protein 38 | | |
| | | | | Auxiliary | | DESCRIPTION: RecName: Full = Leucine-rich repeat- |
| | | | | protein of | | containing protein 38; AltName: Full = BK channel |
| | | | | voltage and | | auxilliary gamma subunit LRRC38; Flags: Precursor; |
| | | | | ca++activated | | |
| | | | | potassium | | |
| | | | | channel | | |
| | | | | SUBUNIT: | | FUNCTION: Auxiliary protein of the large-conductance, |
| | | | | Interacts | | voltage and calcium-activated potassium channel (BK alpha). |
| | | | | with | | Modulates gating properties by producing a marked shift in |
| | | | | KCNMA1 | | the BK channel's voltage dependence of activation in the |
| | | | | (alignment | | hyperpolarizing direction, and in the absence of calcium. |
| | | | | #2) | | |

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. The use of one or more Transposable Element remnant (TEr) nucleic acid sequences and promoter and promoter-proximal non-processive transcripts (NPtx) sequences of pathway hub genes and/or their associated (in cis or trans) lncRNA, to augment, alter, block or otherwise modify the transcription of genes that contain high identity (but not necessarily identical) nucleic acid sequences.

Clause 2. A method to identify the DNA sequences of Clause 1.

Clause 3. Specific nucleic acid sequences that can be utilized to block, disrupt or augment one or more of the following pathways: 1) epithelial to mesenchymal transition, 2) phospholipid signaling pathway, 3) myogenesis, 4) Parkinson's Disease-associated pathways, 5) stress-mediated fat metabolism, 6) CD4+ T cell activation and HIV binding, wherein the nucleic acid sequences have sequence identifiers from SEQ ID NO:1-SEQ ID NO:3918.

Clause 4. The nucleic acid sequences of Clause 3, modified by the addition of nuclear localization signals and/or “bar codes” and/or other nucleic acid identifiers and/or other synthetic modifiers.

Clause 5. A composition comprising a nucleic acid sequences of Clauses 3 or 4, and delivery molecule comprising viral vectors, nanoparticles or extracellular vesicles.

Clause 6. The use of sequences of Clause 3 as diagnostic or prognostic tools.

Clause 7. The use of sequences of Clause 3 to define a tumor or disease “signature”.

Clause 8. The use of sequences of Clause 3 for inhibition of epithelial to mesenchymal transition and/or maintaining tumor heterogeneity.

Clause 7. The use of sequences Clause 3 for the identification of cell function-specific pathways and/or for staging specific differentiation or developmental stages in cells, tissue and/or tissue samples.

Clause 8. The use of sequences Clause 3 to trigger or modify stem cells to differentiate into a tissue and/or cell type-of-interest and/or inducing specific differentiation or developmental stages in cells, tissue and/or tissue samples.

Clause 9. The use of TEr/NPtx-specific stands that are discovered by “pulled down” techniques, including but not restricted to Chromatin Immunoprecipitation for example, for the further identification of a specific genomic pathway or network.

Clause 10. A synthetic nucleic acid comprising one or more of a transposon remnant, a promoter and/or a promoter-proximal non-processive transcript, selected to modulate gene-to-gene transcriptional signaling within a given functional pathway.

Clause 11. The synthetic nucleic acid of Clause 10, to further modulate transcription of a plurality of genes within a network.

Clause 12. The synthetic nucleic acid of any of Clause 10-11, wherein the synthetic nucleic acid has a sequence that aligns with high identity to transcriptional regulatory regions of genes participating in the given functional pathway.

Clause 13. The synthetic nucleic acid of any of Clauses 10-12, wherein high identity is defined based on high identity BLAT₂₀₁₃ alignment, or other “in silico” genomic alignment algorithm

Clause 14. The synthetic nucleic acid of any of Clauses 10-13, further comprising nuclear localization signals and/or “bar codes” and/or other nucleic acid identifiers and/or other synthetic modifiers.

Clause 15. The synthetic nucleic acid of any of Clause 10-14, wherein the given functional pathway is selected from the group consisting of epithelial to mesenchymal transition pathway, phospholipid signaling pathway, myogenesis pathway, stress-mediated fat metabolism pathway, CD4+ T-cell activation and HIV binding pathway, and a Parkinson's Disease-associated pathway.

Clause 16. A method of modulating epigenetic communication between genes coordinating specific pathways, the method comprising:
delivering one or more synthetic nucleic acids as in any of Clause 10-15 to a sample of cells and/or a tissue and/or an animal model of disease and/or a human clinical trial.

Clause 17. The method of Clause 16, wherein delivering the one or more synthetic nucleic acids comprises delivery a delivery vehicle comprising the one or more nucleic acids, and nanoparticles or extracellular vesicles.

Clause 18. The method of any of Clauses 16-17, wherein modulating the epigenetic communication between genes coordinating specific pathways comprises ablate, inhibit or augment the transcription, translation or expression of one or more of functionally-linked genes.

Clause 19. The method of any of Clauses 16-18, further comprising determining a set of functionally-linked genes.

Clause 20. The method of any of Clauses 16-19, wherein determining the set of functionally-linked genes comprises:
(a) selecting a transposon remnant, a promoter, or a promoter-proximal non-processive transcript of a first index gene from a given functional pathway;
(b) identifying, using a computer implemented sequence alignment algorithm implemented by a processor, transposon remnant sequences from a set of genes, having a high homology/identity with the selected transposon remnant, promoter, or promoter-proximal non-processive transcript;
(c) determining, by the processor, a genomic position of the transposon remnant sequences with highest sequence identity with the selected transposon remnant, promoter, or promoter-proximal non-processive transcript;
(d) in response to a determination that the genomic position of a given identified transposon remnant sequence is within a gene regulatory region of a first gene among the set of genes, tabulating, by the processor, function of the first gene;
(e) repeating (a)-(d) for identified transposon remnant sequences that are in cis to the selected transposon remnant, promoter, or promoter-proximal non-processive transcript to determine transposon remnant sequences of genes connected to the first index gene; and
(f) repeating (a)-(e) with transposon remnant sequences of genes, among the set of genes, connected to the first index gene to determine a group of genes forming the given functional pathway.

Clause 21. The method of any of Clauses 16-20, further comprising: (g) repeating (a)-(f) for a second index gene.

Clause 22. A method of determining a network of genes, the method comprising the steps of:
(a) selecting a transposon remnant, a promoter, or a promoter-proximal non-processive transcript of a first index gene from a given functional pathway;
(b) identifying, using a computer implemented sequence alignment algorithm implemented by a processor, transposon remnant sequences from a set of genes, having at least 75% homology with the selected transposon remnant, promoter, or promoter-proximal non-processive transcript;
(c) determining, by the processor, a genomic position of the transposon remnant sequences with highest sequence identity with the selected transposon remnant, promoter, or promoter-proximal non-processive transcript;
(d) in response to a determination that the genomic position of a given identified transposon remnant sequence is within a gene regulatory region of a first gene among the set of genes, tabulating, by the processor, function of the first gene;
(e) repeating (a)-(d) for identified transposon remnant sequences that are in cis to the selected transposon remnant, promoter, or promoter-proximal non-processive transcript to determine transposon remnant sequences of genes connected to the first index gene; and
(f) repeating (a)-(e) with transposon remnant sequences of genes, among the set of genes, connected to the first index gene to determine a group of genes forming the given functional pathway.

Clause 23. The method of Clause 22, further comprising: (g) repeating (a)-(f) for a second index gene.

Clause 24. The method of any of Clauses 22-23, wherein in response to a determination that the group of genes determined for the second index gene is different from the group of genes for the first index gene, determining that second index gene is from a functional pathway different from that of the given functional pathway.

Clause 25. The method of any of Clauses 22-24, wherein the selected transposon remnant, promoter, or promoter-proximal non-processive transcript includes one or more of a from one or more of a transcribed transposon remnant, an ancient transposon remnant, a conserved transposon remnant, a promoter region, an enhancer region, promoter-proximal region, 5′ untranslated region; 3′ untranslated region, a first intron proximal to a transcription start site, and a non-processive transcript region in regulator region or a first intron proximal to a promoter.

Clause 26. The method of any of Clauses 22-25, wherein the first index gene is selected from 2013 UCSC genome or other human genome database.

Clause 27. The method of any of Clauses 22-26, wherein the computer implemented sequence alignment algorithm is BLAT 2013 or other genomic alignment algorithm.

Clause 28. The method of any of Clauses 22-27, wherein the given functional pathway is selected from the group consisting of: epithelial to mesenchymal transition pathway, phospholipid signaling pathway, myogenesis pathway, stress-mediated fat metabolism pathway, CD4+ T-cell activation and HIV binding pathway, and a Parkinson's Disease-associated pathway.

Clause 29. The method of any of Clause 22-28, wherein identifying transposon remnant sequences from a set of genes comprises identifying transposon remnant sequences having high homology/identity with the selected transposon remnant, promoter, or promoter-proximal non-processive transcript.

Clause 30. A method for inducing specific differentiation or developmental stages in cells, the method comprising:
determining a group of genes forming a given functional pathway using the method of any of Clauses 22-29;
delivering one or more synthetic nucleic acids comprising one or more of a transposon remnant, a promoter and/or a promoter-proximal non-processive transcript, and selected to modulate gene-to-gene transcriptional signaling within the given functional pathway,
wherein the given functional pathway is associated with the specific differentiation or developmental stages in cells.

Clause 31. The method of Clause 30, wherein the one or more synthetic nucleic acids have a sequence that aligns with high identity to transcriptional regulatory regions of genes participating in the given functional pathway.

Clause 32. The method of any of Clauses 30-31, wherein high identity is defined based on BLAT₂₀₁₃ or other genomic alignment algorithm.

Clause 33. The method of any of Clauses 30-32, wherein the synthetic nucleic acid has a sequence selected from top ten or more BLAT₂₀₁₃ alignments.

Clause 34. The method of any of Clauses 30-33, wherein the one or more synthetic nucleic acids further comprise nuclear localization signals and/or “bar codes” and/or other nucleic acid identifiers and/or other synthetic modifiers.

Clause 35. The method of any of Clauses 30-34, wherein delivering the one or more synthetic nucleic acids comprises delivery a delivery vehicle comprising the one or more nucleic acids, and nanoparticles or extracellular vesicles or other delivery vehicle.

Clause 36. The method of any of Clauses 30-35, further comprising modulating the epigenetic communication between the group of genes forming the given functional pathway.

Clause 37. The method of any of Clauses 30-36, wherein modulating the epigenetic communication comprises one or more of ablating, inhibiting or augmenting the transcription, translation or expression of one or more of functionally-linked genes.

Clause 38. The method of any of Clauses 30-37, further comprises delivering the Transposable Element remnant (TEr) nucleic acid sequences and promoter and promoter-proximal non-processive transcripts (NPtx) sequences of pathway hub genes and/or their associated (in cis or trans) lncRNA, to augment, alter, block or otherwise modify the transcription of genes that contain high identity nucleic acid sequences being selected to ablate, inhibit or augment the transcription, translation or expression of one or more of functionally-linked genes.

Clause 39. The method of any of Clause 30-38, further comprising delivering an oligonucleotide selected to ablate, inhibit or augment the transcription, translation or expression of one or more of functionally-linked genes.

Clause 40. A method to identify the DNA sequences of Clause 1 employing any of the steps of any of the preceding claims.

The foregoing description is provided to enable a person skilled in the art to practice the various configurations described herein. While the invention has been particularly described with reference to the various figures and configurations, it should be understood that these are for illustration purposes only and should not be taken as limiting the scope of the invention.

There may be many other ways to implement the invention. Various functions and elements described herein may be partitioned differently from those shown without departing from the scope of the invention. Various modifications to these configurations will be readily apparent to those skilled in the art, and generic principles defined herein may be applied to other configurations. Thus, many changes and modifications may be made to the invention, by one having ordinary skill in the art, without departing from the scope of the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the invention, and are not referred to in connection with the interpretation of the description of the invention. All structural and functional equivalents to the elements of the various configurations described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

1. A single- or double-stranded synthetic nucleic acid, comprising a transposon remnant sequence, a promoter non-processive transcript sequence and/or a promoter-proximal non-processive transcript sequence, wherein the transposon remnant is a transposon that is no longer capable of transposition, wherein the synthetic nucleic acid augments, alters or blocks transcription of one or more genes containing high identity DNA sequences thereby modulating gene-to-gene transcriptional signaling within a given functional pathway.

2. The synthetic nucleic acid of claim 1, wherein the one or more genes containing high identity nucleic acid sequences are among a group of genes forming the given functional pathway.

3. The synthetic nucleic acid of claim 2, wherein the synthetic nucleic acid has a sequence that aligns with high identity to transcriptional regulatory regions of genes participating in the given functional pathway.

4. The synthetic nucleic acid of claim 3, wherein high identity is defined based on high identity BLAT₂₀₁₃ alignment, or other “in silico” genomic alignment algorithm

5. The synthetic nucleic acid of claim 2, further comprising nuclear localization signals and/or “bar codes” and/or other nucleic acid identifiers and/or other synthetic modifiers.

6. The synthetic nucleic acid of claim 2, wherein the given functional pathway is selected from the group consisting of: epithelial to mesenchymal transition pathway, phospholipid signaling pathway, myogenesis pathway, stress-mediated fat metabolism pathway, CD4+ T-cell activation and HIV binding pathway, and a Parkinson's Disease-associated pathway.

7.-42. (canceled)

43. The synthetic nucleic acid of claim 1, wherein the transposon remnant sequence is not otherwise functional as a transcription factor binding site, primer binding site, small RNA of previously defined function or coding sequence.
